(19) United States
(12) Patent Application Publication
(10) Pub. No.: US 2021/0310044 A1
(43) Pub. Date: Oct. 7, 2021

(54) ANTIBODY OR APTAMER CONJUGATED-POLYNUCLEOTIDES AND DETECTION METHODS AND MICROFLUIDICS DEVICES USING THE SAME

(71) Applicant: Autonomous Medical Devices Inc., Inglewood, CA (US)

(72) Inventor: Roger D. Kornberg, Atherton, CA (US)

(73) Assignee: Autonomous Medical Devices Inc., Inglewood, CA (US)

(21) Appl. No.: 17/266,058

(22) PCT Filed: Oct. 12, 2019
(86) PCT No.: PCT/US2019/055999

§ 371 Date: Feb. 4, 2021

Related U.S. Application Data
(60) Provisional application No. 62/744,895, filed on Oct. 12, 2018.
(60) Provisional application No. 62/886,759, filed on Aug. 14, 2019.

(51) Int. Cl. C12Q 1/6804; G01N 33/53

(52) U.S. Cl. CPC C12Q 1/6804; G01N 33/5302

(57) ABSTRACT

The disclosure relates to antibody conjugates comprising an antibody linked to a polynucleotide, aptamer conjugates comprising an aptamer linked to a polynucleotide, and methods for detecting a marker or several markers in a sample by using said antibody and aptamer conjugates. The present disclosure also relates to microfluidics devices for detecting markers in a sample. The present disclosure further relates to methods for detecting a microorganism or several microorganisms in a sample and a microfluidics device to be used to detect such microorganisms.

### CROSS-REFERENCE TO RELATED APPLICATIONS

This application claims priority from United States Provisional Applications No. 62/744,895, filed Oct. 12, 2018 and No. 62/886,759, filed Aug. 14, 2019, the contents of which are hereby incorporated by reference in their entirety.

### BACKGROUND OF THE DISCLOSURE

Markers, such as biomarkers, are tools for the diagnosing, monitoring, and screening a number of diseases. Biomarkers can also be used to detect disease risk factors allowing the physician to recommend or prescribe more intensive monitoring or testing of a patient. In many cases, it is possible to diagnose diseases, such as cancer or rheumatic diseases, via determining concentrations of specific biomarkers in blood, i.e., a marker profile. For such applications based on the detection of multiple biomarkers or one of a plurality of biomarkers in one sample, a cost-effective and rapid analysis system with small sample consumption is required.

The two hallmarks of a diagnostic biomarker analysis system are sensitivity and specificity. Sensitivity refers to the percentage of patients with a disease who will test positive in the assay. False negative results dilute the sensitivity of an assay. Specificity refers to the percentage of patients without disease who test as negative in the assay. False positive results dilute the specificity of a diagnostic assay. Although both are extremely important, low sensitivity in a diagnostic assay for cancer can be life threatening if false negative results prevent individuals with cancer from receiving timely treatment.

Microbial and viral identification usually rely on conventional approaches of plating and culture methods, as well as on biochemical testing, microscopy, etc. Over the last 20 years, many new methods have been developed, including immunological methods, polymerase chain reaction (PCR) and biosensors (Deisingh, A. K.; Thompson, M. Biosensors for the detection of bacteria. Can. J. Microbiol. 2004; 50, 69-77). Plating and culture methods often fail to provide the required specificity and sensitivity and can take up to 7 days to complete. PCR, although very specific and suitable for screening purposes, still fails to produce accurate results when enumeration of viable cells is needed (March, C. et al. J. Immunol. Methods 2005, 303, 92-104). Immunological detection with antibodies is perhaps the most successful technology employed for the detection of cells, spores, viruses and toxins alike (Iqbal, S. S. et al. Biosens. Bioelectron. 2000, 15, 549-578). The availability of monoclonal antibodies, together with the emergence of recombinant antibody phage display technology, has made immunological detection of microbial contamination more sensitive, specific, reproducible and reliable. These technologies, when incorporated in biosensors, significantly shorten the assay time and improve the analytical performance of pathogen detection.

Enzyme-linked immunosorbent assays (ELISA) and radioimmunoassays are generally regarded as the gold standards in terms of sensitivity and selectivity, and a number of research groups are attempting to implement such assays with microfabricated devices.

Antibody conjugates are widely used as diagnostics and imaging reagents. However, many such conjugates lose sensitivity and specificity due to nonspecific labeling techniques. The ability to detect very rare cells or markers at low concentrations in the blood with accuracy and sensitivity is still a significant problem for molecular diagnostics. Typical protein detection methods such as ELISA are often not sensitive enough to detect low concentrations of important biological markers such as troponin, prostate-specific antigen, or viral coat proteins.

There is a need for convenient and portable methods and devices for the detection of markers or microorganisms, including biomarkers and environmental markers, especially more sensitive, specific, and robust sensors. See, e.g., Kaisti, M. Biosensors and Bioelectronics, 2017, vol. 98:437-448, incorporated by reference herein in its entirety. Interactions involving macromolecules, such as antibodies, occur relatively slowly, on the order of 10⁵ specific binding events per second. By contrast, binding of ions to counter ions occurs much more rapidly, on the order of 10¹⁰ or more events per second. The detection of ions in solution, however, is complicated by the screening of detectors from such molecules by oppositely charged ions and other unrelated ions in the solution. See, e.g., Kaisti, M. Biosensors and Bioelectronics, 2017, vol. 98:437-448, incorporated by reference herein in its entirety. Accordingly, there is a need in the art for improving the selective, sensitive and robust detection of markers or microorganisms, maintaining a high specificity.

### SUMMARY OF THE DISCLOSURE

The present disclosure provides binding member-nucleic acid conjugates, which can be used to detect a marker or several markers in a sample, as well as a microorganism, such as a virus or multiple viruses. The present disclosure also provides methods for rapidly detecting markers, including markers at low concentrations, while maintaining high sensitivity and specificity. The present method provides an extraordinary sensitivity to detect low concentrations of markers in samples.

A first aspect of the present disclosure provides an antibody conjugate comprising an antibody linked to a polynucleotide, wherein the polynucleotide comprises a first binding nucleotide sequence that is capable of binding to a capture DNA binding domain (DBD) but incapable of binding to a detection DBD and a second binding nucleotide sequence that is capable of binding to the detection DBD but incapable of binding to the capture DBD. This aspect of the disclosure also provides an aptamer linked to a polynucleotide, wherein the polynucleotide comprises a first binding nucleotide sequence that is capable of binding to a capture DNA binding domain (DBD) but incapable of binding to a detection DBD and a second binding nucleotide sequence that is capable of binding to the detection DBD but incapable of binding to the capture DBD. In some embodiments, the aptamer is an oligonucleotide. Optionally, the aptamer comprises DNA residues. In some embodiments, the aptamer comprises RNA residues. Optionally, the oligonucleotide is single-stranded.

In some embodiments, the capture DBD comprises a helix-turn-helix motif. Optionally, the capture DBD is selected from the group consisting of MAT α1, MAT α2, MAT a1, Antennapedia, Ultrabithorax, Engrailed, Paired, Fushi tarazu, HOX, Unc86, Oct1, Oct2, Oct4, hRFX1, Pit, TCF-1, SRY, TrpR, RuvC, LexA, Lac I repressor, Bacteriophage Lambda Cro repressor, Bacteriophage 434 C1 and Cro repressors, P22 C2 repressor, and Bacteriophage Mu Ner protein, including variant forms of helix-turn-helix motifs. The capture DBD may comprise a zinc-finger motif. Optionally, the capture DBD is selected from the group consisting of Zif268, SWI5, SIP1, FOG, Msn2p, A20, Klf4, Mac1, steroid receptors, the yeast transcriptional activator GAL4, and Krüppel and Hunchback, including variant forms of zinc-finger motifs. In some embodiments, the capture DBD comprises a basic leucine zipper motif. Optionally, the capture DBD is selected from the group consisting of c-Fos/c-Jun, AP-1 Fos/Jun, CREB, ATF1, ATF2, ATF4, ATF5, ATF6, ATF7, BACH1, BACH2, BATF, BATF2, CEBPA, CEBPB, CEBPD, CEBPE, CEBPG, CEBPZ, CREB1, CREB3, CREB3L1, CREB3L2, CREB3L3, CREB3L4, CREB5, CREBL1, CREM, E4BP4, FOSL1, FOSL2, GCN4, JUN, JUNB, JUND, MAFA, MAFB, NFE2, NFE2L2, NFE2L3, SNFT, XBP1 OPAQUE, NFE2L2, and Bzip Maf, including variant forms of basic leucine zipper motifs. The capture DBD may comprise a helix-loop-helix motif Optionally, the capture DBD is selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, BHLHE41, CLOCK, BMAL-1-CLOCK, EPAS1, FERD3L, FIGLA, HAND1, HAND2, HES1, HES2, HES3, HES4, HES5, HES6, HES7, HEY1, HEY2, HIF, HIF1A, ICE1, ID1, ID2, ID3, ID4, KIAA2018, LYL1, MASH1, MATH2, MAX, MESP1, MESP2, MIST1, MITF, MLX, MLXIP, MLXIPL, MNT, MOP5, MSC, MSGN1, MXD1, MXD3, MXD4, MXI1, C-Myc, N-Myc, MyoD, MYC, MYCL1, MYCL2, MYCN, MYF5, MYF6, MYOD1, MYOG, NCOA1, NCOA3, Neurogenins, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, Pho4, PTF1A, SCL, Scleraxis, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, USF2, Beta2/NeuroD1, Daughterless, Achaete-scute (T3), E12 and E47, including variant forms of helix-loop-helix motifs.

In some embodiments, the detection DBD comprises a helix-turn-helix motif. Optionally, the detection DBD is selected from the group consisting of MAT α1, MAT α2, MAT a1, Antennapedia, Ultrabithorax, Engrailed, Paired, Fushi tarazu, HOX, Unc86, Oct1, Oct2, Oct4, hRFX1, Pit, TCF-1, SRY, TrpR, RuvC, LexA, Lac I repressor, Bacteriophage Lambda Cro repressor, Bacteriophage 434 C1 and Cro repressors, P22 C2 repressor, and Bacteriophage Mu Ner protein, including variant forms of helix-turn-helix motifs. The capture DBD may comprise a zinc-finger motif. Optionally, the capture DBD is selected from the group consisting of Zif268, SWI5, SIP1, FOG, Msn2p, A20, Klf4, Mac1, steroid receptors, the yeast transcriptional activator GAL4, and Krüppel and Hunchback, including variant forms of zinc-finger motifs. In some embodiments, the capture DBD comprises a basic leucine zipper motif. Optionally, the capture DBD is selected from the group consisting of c-Fos/c-Jun, AP-1 Fos/Jun, CREB, ATF1, ATF2, ATF4, ATF5, ATF6, ATF7, BACH1, BACH2, BATF, BATF2, CEBPA, CEBPB, CEBPD, CEBPE, CEBPG, CEBPZ, CREB1, CREB3, CREB3L1, CREB3L2, CREB3L3, CREB3L4, CREB5, CREBL1, CREM, E4BP4, FOSL1, FOSL2, GCN4, JUN, JUNB, JUND, MAFA, MAFB, NFE2, NFE2L2, NFE2L3, SNFT, XBP1 OPAQUE, NFE2L2, and Bzip Maf, including variant forms of basic leucine zipper motifs. The capture DBD may comprise a helix-loop-helix motif Optionally, the capture DBD is selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, BHLHE41, CLOCK, BMAL-1-CLOCK, EPAS1, FERD3L, FIGLA, HAND1, HAND2, HES1, HES2, HES3, HES4, HES5, HES6, HES7, HEY1, HEY2, HIF, HIF1A, ICE1, ID1, ID2, ID3, ID4, KIAA2018, LYL1, MASH1, MATH2, MAX, MESP1, MESP2, MIST1, MITF, MLX, MLXIP, MLXIPL, MNT, MOP5, MSC, MSGN1, MXD1, MXD3, MXD4, MXI1, C-Myc, N-Myc, MyoD, MYC, MYCL1, MYCL2, MYCN, MYF5, MYF6, MYOD1, MYOG, NCOA1, NCOA3, Neurogenins, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, Pho4, PTF1A, SCL, Scleraxis, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, USF2, Beta2/NeuroD1, Daughterless, Achaete-scute (T3), E12 and E47, including variant forms of helix-loop-helix motifs.

In some embodiments, the first binding nucleotide sequence is separated from the second binding nucleotide sequence by a linker sufficient to avoid steric hindrance between the capture DBD and the detection DBD. Optionally, the linker is at least 10 nucleotides long. Optionally, the linker is in the range of 10 to 50 nucleotides long, or in the range of 15 to 40 nucleotides long, or in the range of 20 to 35 nucleotides long, or in the range of 25-30 nucleotides long, or around 20 nucleotides long. The polynucleotide may further comprise a first amplification nucleotide sequence and a second amplification nucleotide sequence, wherein the first binding nucleotide sequence and the second binding nucleotide sequence are between the first amplification nucleotide sequence and the second amplification nucleotide sequence. In some embodiments, the first amplification nucleotide sequence and the second amplification nucleotide sequence are suitable for isothermal amplification. Optionally, the isothermal amplification is Loop Mediated Isothermal Amplification (LAMP). Optionally, the first amplification nucleotide sequence and the second amplification nucleotide sequence are suitable for PCR amplification.

In some embodiments, the polynucleotide is capable of being released from the antibody or aptamer upon antigen binding. Optionally, the polynucleotide is released by cleavage of a disulfide bond. The antibody or aptamer may be linked to the polynucleotide through a protein. Optionally, the protein is selected from the group consisting of protein A, protein G, and protein L.

A second aspect of the present disclosure provides a method for detecting a marker in a sample, the method comprising: (a) contacting the sample with the antibody conjugate or the aptamer conjugate according to the first aspect of the disclosure, wherein the antibody portion or the aptamer portion of the conjugate binds to the marker; (b) amplifying the polynucleotide portion of the antibody conjugate or aptamer conjugate; (c) binding the first nucleotide binding sequence of the amplified polynucleotide to a capture DNA binding domain (DBD), wherein the capture DBD is affixed to a scaffold; (d) binding the second nucleotide binding sequence of the amplified polynucleotide to a detection DBD, wherein the detection DBD is affixed to a detectable label; and (e) detecting the detectable label.

In some embodiments, the marker is a biomarker, an environmental marker, an allergen, or a microorganism. Optionally, the microorganism is selected from the group consisting of a bacterium, a fungus, an archaeon, an alga, a protozoan and a virus. The sample may be an environmental sample, a food sample, or a sample obtained from a subject.

In some embodiments, the polynucleotide is released upon binding of the marker to the antibody conjugate or aptamer conjugate. In other embodiments, the marker is bound to a capture molecule, wherein the capture molecule is affixed to a scaffold or capable of being affixed to a scaffold. The capture molecule may be a capture antibody. In some embodiments, the capture molecule is a capture aptamer.

Optionally, the method further comprises the step of washing the antibody conjugate-bound marker or the aptamer conjugate-bound marker before the amplification step.

In some embodiments, the method further comprises the step of washing the amplified polynucleotide bound to the capture DBD prior to binding the detection DBD. In other embodiments, the method further comprising the step of washing the amplified polynucleotide bound to the detection DBD prior to detecting the detectable label. The amplifying step within the method may be performed by isothermal amplification or may be performed by PCR amplification. Optionally, the amplification comprises the step of binding a first amplification primer to a first amplification sequence in the polynucleotide portion of the antibody conjugate and binding a second amplification primer to a second amplification sequence in the polynucleotide portion of the antibody conjugate.

In other embodiments, the detectable label is capable of being detected by a surface acoustic wave device. Optionally, the detectable label is selected from the group of a charged particle, a magnetic particle, a metal particle and a spore. In some embodiments, the detectable label is capable of being detected by a field effect transistor. Optionally, the detectable label is selected from the group of a fluorescent label, an enzymatic label and a radioactive label.

In some embodiments, the method is performed on a microfluidics device. In other embodiments, the marker is present in the sample at a concentration that cannot be detected without signal amplification.

A third aspect of the present disclosure provides a method of detecting one of a plurality of markers in a sample, the method comprising: (a) contacting the sample with a first antibody conjugate and a second antibody conjugate, wherein the first antibody conjugate and second antibody conjugate are antibody conjugates according to the first aspect of the disclosure, wherein the antibody portion of the first antibody conjugate binds a different marker than the antibody portion of the second antibody conjugate, wherein the first binding nucleotide sequence of the polynucleotide portion of the first antibody conjugate binds to a first capture DNA binding domain (DBD) and the first binding nucleotide sequence of the polynucleotide portion of the second antibody conjugate binds to a second capture DBD, and wherein the second binding nucleotide sequence of the polynucleotide portion of the first antibody conjugate binds to a first detection DBD, and the second binding nucleotide sequence of the polynucleotide portion of the second antibody conjugate binds to the second detection DBD; (b) amplifying the polynucleotide portion of the marker-bound antibody conjugate; (c) binding the first nucleotide binding sequence of the amplified polynucleotide to a capture DBD, wherein the capture DBD binds to the first binding nucleotide sequence of the first and second antibody conjugates and is affixed to a scaffold; (d) binding the second binding nucleotide sequence of the amplified polynucleotide to a first detection DBD, wherein the first detection DBD binds to the second binding nucleotide sequence of the first antibody conjugate and is affixed to a first detectable label; (e) performing a first detection step to detect the first detectable label; (f) binding the amplified polynucleotide to a second detection DBD, wherein the second detection DBD binds to the second binding nucleotide sequence of the second antibody conjugate and is affixed to a second detectable label; and (g) performing a second detection step to detect the second detectable label. This aspect also includes a method comprising: (a) contacting the sample with a first aptamer conjugate and a second aptamer conjugate, wherein the first aptamer conjugate and second aptamer conjugate are aptamer conjugates according to the first aspect of the disclosure, wherein the aptamer portion of the first aptamer conjugate binds a different marker than the aptamer portion of the second aptamer conjugate, wherein the first binding nucleotide sequence of the polynucleotide portion of the first aptamer conjugate binds to a first capture DNA binding domain (DBD) and the first binding nucleotide sequence of the polynucleotide portion of the second aptamer conjugate binds to a second capture DBD, and wherein the second binding nucleotide sequence of the polynucleotide portion of the first aptamer conjugate binds to a first detection DBD, and the second binding nucleotide sequence of the polynucleotide portion of the second aptamer conjugate binds to the second detection DBD; (b) amplifying the polynucleotide portion of the marker-bound aptamer conjugate; (c) binding the first nucleotide binding sequence of the amplified polynucleotide to a capture DBD, wherein the capture DBD binds to the first binding nucleotide sequence of the first and second aptamer conjugates and is affixed to a scaffold; (d) binding the second binding nucleotide sequence of the amplified polynucleotide to a first detection DBD, wherein the first detection DBD binds to the second binding nucleotide sequence of the first aptamer conjugate and is affixed to a first detectable label; (e) performing a first detection step to detect the first detectable label; (f) binding the amplified polynucleotide to a second detection DBD, wherein the second detection DBD binds to the second binding nucleotide sequence of the second aptamer conjugate and is affixed to a second detectable label; and (g) performing a second detection step to detect the second detectable label. This aspect also includes methods using an antibody conjugate that binds a first marker and does not bind a second marker and an aptamer conjugate that binds the second marker and does not bind the first marker.

In some embodiments, the first and second capture DBDs bind the same first binding nucleotide sequence. Optionally, the first and second capture DBDs are the same DBD. Optionally, the first and second capture DBDs bind different first binding nucleotide sequences.

In some embodiments, the polynucleotide portion of each antibody conjugate or aptamer conjugate is released upon binding of the marker to the antibody conjugate aptamer conjugate. In other embodiments, each of the plurality of markers is bound to a capture molecule, wherein each capture molecule is affixed to a scaffold or capable of being affixed to a scaffold. In some embodiments, the capture molecule is a capture antibody. Optionally, the capture molecule is a capture aptamer.

In some embodiments, the method further comprises the step of washing the antibody conjugate-bound markers or the aptamer conjugate-bound markers before the amplification step. The first detectable label and the second detectable label may be different, and the marker is detected by which detectable label is present. Alternatively, the first detectable label and the second detectable label may be the same, and the marker is detected by whether the detectable label is present in the first detection step or the second detection step.

In some embodiments, the method further comprising the step of washing the amplified polynucleotide bound to the capture DBD prior to binding the detection DBD. In other embodiments, the method further comprises the step of washing the amplified polynucleotide bound to the first detection DBD prior to first detection step. In other embodiments, the method further comprises the step of washing the amplified polynucleotide bound to the capture DBD after the first detection step prior to binding the second detection DBD. In other embodiments, the method further comprises the step of washing the amplified polynucleotide bound to the second detection DBD prior to second detection step.

In some embodiments, the amplifying step may be performed by isothermal amplification. Alternatively, the amplifying step is performed by PCR amplification. In other embodiments, the amplification comprises the step of binding a first amplification primer to a first amplification sequence in the polynucleotide portion of the antibody conjugate or the aptamer conjugate and binding a second amplification primer to a second amplification sequence in the polynucleotide portion of the antibody conjugate or the aptamer conjugate. Optionally, the first amplification sequence in the polynucleotide portion of the first antibody conjugate or the first aptamer conjugate is the same as the first amplification sequence in the polynucleotide portion of the second antibody conjugate or the second aptamer conjugate. Optionally, the second amplification sequence in the polynucleotide portion of the first antibody conjugate or the first aptamer conjugate is the same as the second amplification sequence in the polynucleotide portion of the second antibody conjugate or the second aptamer conjugate.

In some embodiments, the first detectable label is capable of being detected by a surface acoustic wave device. Optionally, the first detectable label is selected from the group of a magnetic particle, a metal particle, a particle of 1 pg or greater and a spore. In other embodiments, the first detectable label is capable of being detected by a field effect transistor. Optionally, the first detectable label is selected from the group of a magnetic particle, a metal particle and a lipid vesicle comprising a charged solution. Optionally, the charged solution is an ionic solution. Optionally, the first detectable label is selected from the group of a magnetic particle, a metal particle and a lipid vesicle containing an ionic solution in the aqueous compartment of the vesicle. Optionally, the charged solution is an ionic solution. In other embodiments, the first detectable label is selected from the group of a fluorescent label, an enzymatic label and a radioactive label.

In some embodiments, the second detectable label is capable of being detected by a surface acoustic wave device. Optionally, the second detectable label is selected from the group of a charged particle, a magnetic particle, a metal particle and a spore. In other embodiments, the second detectable label is capable of being detected by a field effect transistor. Optionally, the second detectable label is selected from the group of a magnetic particle, a metal particle and a lipid vesicle comprising a charged solution. Optionally, the charged solution is an ionic solution. In other embodiments, the second detectable label is selected from the group of a fluorescent label, an enzymatic label and a radioactive label.

In some embodiments, the method is performed on a microfluidics device.

Optionally, the first detection DBD is released from a first channel in the microfluidics device. Optionally, the second detection DBD is released from a second channel in the microfluidics device. Optionally, the release of the first detection DBD from the first channel and the first detection step occur before the release of the second detection DBD from the second channel.

In some embodiments, the method further comprises: (1) contacting the sample with one or more additional antibody conjugates, wherein each of the one or more additional antibody conjugates is an antibody conjugate according to the first aspect of the disclosure, wherein each of the antibody portion of the one or more additional antibody conjugates binds a different marker than the antibody portion of the first antibody conjugate, the antibody portion of the second antibody conjugate and the antibody portion of the any other additional antibody conjugate; wherein the first binding nucleotide sequence of each of the one or more additional antibody conjugates binds to the same capture DBD as the first binding nucleotide sequence of the first and second antibody conjugates, wherein the second nucleotide binding sequence of each of the one or more additional antibody conjugates binds to a different DBD than the second nucleotide binding sequence of the first antibody conjugate, the second antibody conjugate and any other additional antibody conjugates; (2) binding the amplified polynucleotide to one or more additional DBDs, wherein each of the one or more additional DBDs binds to the second binding nucleotide sequence of the one or more additional antibody conjugates and is affixed to a one or more additional detectable label; and (3) performing one or more additional detection steps to detect the one or more additional detectable labels. Optionally, the method further comprises: (1) contacting the sample with one or more additional aptamer conjugates, wherein each of the one or more additional aptamer conjugates is an aptamer conjugate according to the first aspect of the disclosure, wherein each of the aptamer portion of the one or more additional aptamer conjugates binds a different marker than the aptamer portion of the first aptamer conjugate, the aptamer portion of the second aptamer conjugate and the aptamer portion of the any other additional aptamer conjugate; wherein the first binding nucleotide sequence of each of the one or more additional aptamer conjugates binds to the same capture DBD as the first binding nucleotide sequence of the first and second aptamer conjugates, wherein the second nucleotide binding sequence of each of the one or more additional aptamer conjugates binds to a different DBD than the second nucleotide binding sequence of the first aptamer conjugate, the second aptamer conjugate and any other additional aptamer conjugates; (2) binding the amplified polynucleotide to one or more additional DBDs, wherein each of the one or more additional DBDs binds to the second binding nucleotide sequence of the one or more additional aptamer conjugates and is affixed to a one or more additional detectable label; and (3) performing one or more additional detection steps to detect the one or more additional detectable labels. The method may include the use of at least one additional antibody conjugate and at least one additional aptamer conjugate.

In some embodiments, the marker is present in the sample at a concentration that cannot be detected without signal amplification.

A fourth aspect of the present disclosure provides a microfluidics device comprising: (a) means for receiving a sample; (b) an antibody conjugate, wherein the conjugate comprises an antibody linked to a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD; (c) means for contacting the sample with the antibody conjugate; (d) means for amplifying the polynucleotide portion of the antibody conjugate; (e) the capture DBD affixed to a scaffold; (f) means for contacting the amplified polynucleotide with the capture DBD; (g) the detection DBD attached to a detectable label, wherein the capture DBD and the detection DBD are different; (h) means for contacting the amplified polynucleotide with the detection DBD; and (i) means for detecting the detectable label. This aspect of the disclosure also provides a microfluidics device comprising: (a) means for receiving a sample; (b) an aptamer conjugate, wherein the conjugate comprises an aptamer linked to a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD; (c) means for contacting the sample with the aptamer conjugate; (d) means for amplifying the polynucleotide portion of the aptamer conjugate; (e) the capture DBD affixed to a scaffold; (f) means for contacting the amplified polynucleotide with the capture DBD; (g) the detection DBD attached to a detectable label, wherein the capture DBD and the detection DBD are different; (h) means for contacting the amplified polynucleotide with the detection DBD; and (i) means for detecting the detectable label.

In some embodiments, the antibody conjugate is an antibody conjugate according to the first aspect of the disclosure. Optionally, the aptamer conjugate is an aptamer conjugate according to the first aspect of the disclosure. In other embodiments, the polynucleotide portion of the antibody conjugate or the aptamer conjugate is amplified by isothermal amplification. In other embodiments, the polynucleotide portion of the antibody conjugate or the aptamer conjugate is amplified by PCR. In other embodiments, the microfluidics device further comprises a first amplification primer that binds to a first amplification sequence in the polynucleotide portion of the antibody conjugate or the aptamer conjugate and a second amplification primer that binds to a second amplification sequence in the polynucleotide portion of the antibody conjugate or the aptamer conjugate.

In some embodiments, the means for detecting the detectable label is a surface acoustic wave device. Optionally, the detectable label is selected from the group of a charged particle, a magnetic particle, a metal particle and a spore. In other embodiments, the means for detecting the detectable label is a field effect transistor. Optionally, the detectable label is selected from the group of a magnetic particle, a metal particle, and a lipid vesicle comprising a charged solution in the aqueous compartment. Optionally, the charged solution is an ionic solution. In other embodiments, the means for detecting the detectable label is selected from the group of means for detecting a fluorescent label, means for detecting an enzymatic label and means for detecting a radioactive label.

In some embodiments, the device further comprises means for washing the amplified polynucleotide bound to the capture DBD. In other embodiments, the device further comprises means for washing the amplified polynucleotide bound to the detection DBD.

In some embodiments, the device further comprises means for immobilizing the marker. Optionally, the means for immobilizing the marker is an antibody that binds the marker and is affixed to a solid phase or is capable of being affixed to a solid phase. In other embodiments, the means for immobilizing the marker is an aptamer that binds the marker and is affixed to a solid phase or is capable of being affixed to a solid phase. Optionally, the device further comprises means for washing the immobilized marker.

In some embodiments, the device comprises means for cycling an electric field or a magnetic field.

A fifth aspect of the present disclosure provides a microfluidics device comprising: (a) means for receiving a sample; (b) a first antibody conjugate, wherein the first antibody conjugate comprises a first antibody linked to a first polynucleotide, wherein the first polynucleotide comprises a capture nucleotide sequence that is capable of binding to a capture DNA binding domain (DBD) and is incapable of binding to a first or second detection DBD and a first detection nucleotide sequence that is capable of binding to the first detection DBD and is incapable of binding to the capture DBD or the second detection DBD; (c) a second antibody conjugate, wherein the second antibody conjugate comprises a second antibody linked to a second polynucleotide, wherein the second polynucleotide comprises a capture nucleotide sequence that is capable of binding to the capture DBD and is incapable of binding to the first or second detection DBD and a second detection nucleotide sequence that is capable of binding to the second detection DBD and is incapable of binding to the capture DBD or the first detection DBD; (d) means for contacting the sample with the antibody conjugates; (e) means for amplifying the polynucleotide portions of the first and second antibody conjugates; (f) the capture DBD affixed to a scaffold; (g) means for contacting the amplified polynucleotides with the capture DBD; (h) the first detection DBD attached to a first detectable label, the second detection DBD attached to a second detectable label; (i) means for contacting the amplified polynucleotides with the first and second detection DBDs; and (j) means for detecting the first and second detectable labels. This aspect also provides a microfluidics device comprising: (a) means for receiving a sample; (b) a first aptamer conjugate, wherein the first aptamer conjugate comprises a first aptamer linked to a first polynucleotide, wherein the first polynucleotide comprises a capture nucleotide sequence that is capable of binding to a capture DNA binding domain (DBD) and is incapable of binding to a first or second detection DBD and a first detection nucleotide sequence that is capable of binding to the first detection DBD and is incapable of binding to the capture DBD or the second detection DBD; (c) a second aptamer conjugate, wherein the second aptamer conjugate comprises a second aptamer linked to a second polynucleotide, wherein the second polynucleotide comprises a capture nucleotide sequence that is capable of binding to the capture DBD and is incapable of binding to the first or second detection DBD and a second detection nucleotide sequence that is capable of binding to the second detection DBD and is incapable of binding to the capture DBD or the first detection DBD; (d) means for contacting the sample with the aptamer conjugates; (e) means for amplifying the polynucleotide portions of the first and second aptamer conjugates; (f) the capture DBD affixed to a scaffold; (g) means for contacting the amplified polynucleotides with the capture DBD; (h) the first detection DBD attached to a first detectable label, the second detection DBD attached to a second detectable label; (i) means for contacting the amplified polynucleotides with the first and second detection DBDs; and (j) means for detecting the first and second detectable labels. This aspect also includes a microfluidics device comprising an antibody conjugate and an aptamer conjugate.

In some embodiments, the first antibody conjugate is an antibody conjugate according to the first aspect of the present disclosure. In other embodiments, the second antibody conjugate is an antibody conjugate according to the first aspect of the present disclosure. In some embodiments, the first aptamer conjugate is an aptamer conjugate according to the first aspect of the present disclosure. In other embodiments, the second aptamer conjugate is an aptamer according to the first aspect of the present disclosure. Optionally, the first antibody conjugate or the first aptamer conjugate binds a different marker than the second antibody conjugate or the second aptamer conjugate.

In some embodiments, the polynucleotide portions of the antibody conjugates or the aptamer conjugates are amplified by isothermal amplification. In other embodiments, the polynucleotide portions of the antibody conjugates or the aptamer conjugates are amplified by PCR. In other embodiments, the microfluidics device further comprises a first amplification primer that binds to a first amplification sequence in the polynucleotide portion of the first or second antibody conjugate or the first or second aptamer conjugate and a second amplification primer that binds to a second amplification sequence in the polynucleotide portion of the first or second antibody conjugate or the first or second aptamer conjugate.

In some embodiments, the first detectable signal and the second detectable signal are different. In other embodiments, the first detectable signal and the second detectable signal are the same. The first detection DBD may be stored in a first channel and the second detection DBD may be stored in a second channel.

In some embodiments, the first detection DBD is released from the first channel and the means for detecting the first detectable label is performed prior to the release of the second detection DBD from the second channel.

In some embodiments, the means for detecting the first detectable label is a surface acoustic wave device. Optionally, the first detectable label is selected from the group of a charged particle, a magnetic particle, a metal particle and a spore. In other embodiments, the means for detecting the first detectable label is a field effect transistor. Optionally, the first detectable label is selected from the group of a magnetic particle, a metal particle and a lipid vesicle comprising a charged solution. Optionally, the charged solution is an ionic solution. In other embodiments, the means for detecting the first detectable label is selected from the group of means for detecting a fluorescent label, means for detecting an enzymatic label and means for detecting a radioactive label.

In some embodiments, the means for detecting the second detectable label is a surface acoustic wave device. Optionally, the second detectable label is selected from the group of a charged particle, magnetic particle, a metal particle and a spore. In other embodiments, the means for detecting the second detectable label is a field effect transistor. Optionally, the second detectable label is selected from the group of a charged particle, a magnetic particle, a metal particle and a lipid vesicle comprising a charged solution. Optionally, the charged solution is an ionic solution. In other embodiments, the means for detecting the second detectable label is selected from the group of means for detecting a fluorescent label, means for detecting an enzymatic label and means for detecting a radioactive label.

In some embodiments, the microfluidics device further comprises one or more additional detection DBDs attached to one or more additional detectable labels. Optionally, the one or more additional detectable labels are the same as the first and second detectable labels. Optionally, each of the one or more additional detectable labels is different from the first detectable label, the second detectable label, and any other additional detectable labels.

In some embodiments, the microfluidics device comprises one or more additional antibody conjugates, wherein each of the one or more additional antibody conjugates comprises an antibody linked to one or more additional polynucleotides, wherein each of the one or more additional polynucleotides comprises a capture nucleotide sequence that binds to the capture DBD and does not bind to the first, second, or any additional detection DBD, and an additional detection nucleotide sequence that binds to the one or more additional detection DBDs and does not bind to the capture DBD, the first, second or any other additional detection DBD. In some embodiments, the microfluidics device comprises one or more additional aptamer conjugates, wherein each of the one or more additional aptamer conjugates comprises an aptamer linked to one or more additional polynucleotides, wherein each of the one or more additional polynucleotides comprises a capture nucleotide sequence that binds to the capture DBD and does not bind to the first, second, or any additional detection DBD, and an additional detection nucleotide sequence that binds to the one or more additional detection DBDs and does not bind to the capture DBD, the first, second or any other additional detection DBD. Optionally, the microfluidics device comprises at least one additional antibody conjugate and at least one additional aptamer conjugate.

In some embodiments, the device further comprises means for washing the amplified polynucleotide bound to the capture DBD. In other embodiments, the device further comprises means for washing the amplified polynucleotide bound to the first detection DBD. In other embodiments, the device further comprises means for washing the amplified polynucleotide bound to the second detection DBD. In other embodiments, the device further comprises means for washing the amplified polynucleotide bound to the one or more additional detection DBDs.

In some embodiments, the device further comprises means for immobilizing the marker. Optionally, the means for immobilizing the marker is an antibody that binds the marker and is affixed to a solid phase or is capable of being affixed to a solid phase. In some embodiments, the means for immobilizing the marker is an aptamer that binds the marker and is affixed to a solid phase or is capable of being affixed to a solid phase. Optionally, the device further comprises means for washing the immobilized marker.

In some embodiments, the device comprises means for cycling an electric field or a magnetic field.

A sixth aspect of the present disclosure provides a method for detecting a microorganism in a sample, comprising: (a) amplifying a polynucleotide from the microorganism with a first and second primer, wherein the first primer introduces a first binding sequence capable of binding to a capture DNA binding domain (DBD) and incapable of binding to a detection DBD and the second primer introduces a second binding sequence capable of binding the detection DBD and incapable of binding the capture DBD; (b) binding the first binding sequence of the amplified polynucleotide to the capture DBD, wherein the capture DBD is affixed to a scaffold; (c) binding the second binding sequence of the amplified polynucleotide to the detection DBD, wherein the detection DBD is attached to a detectable label; and (d) detecting the label.

In some embodiments, the microorganism is selected from the group consisting of a bacterium, a fungus, an archaeon, an alga, a protozoan and a virus.

In some embodiments, the method further comprises the step of washing the amplified polynucleotide bound to the capture DBD prior to binding the detection DBD. In other embodiments, the method further comprises the step of washing the amplified polynucleotide bound to the detection DBD prior to detecting the presence of the detectable label.

In some embodiments, the amplifying step is performed by isothermal amplification. Optionally, the amplifying step is performed by PCR amplification.

In some embodiments, the detectable label is capable of being detected by a surface acoustic wave device. Optionally, the detectable label is selected from the group of a charged particle, a magnetic particle, a metal particle and a spore. In other embodiments, the detectable label is capable of being detected by a field effect transistor. Optionally, the detectable label is selected from the group of a charged particle, a magnetic particle, a metal particle, and a lipid vesicle comprising a charged solution. Optionally, the charged solution is an ionic solution. In other embodiments, the detectable label is selected from the group of a fluorescent label, an enzymatic label and a radioactive label.

In some embodiments, the method is performed on a microfluidics device. In other embodiments, the sample is obtained from a subject.

A seventh aspect of the present disclosure provides a method for detecting one of a plurality of microorganisms in a sample, comprising: (a) amplifying a polynucleotide from the plurality of microorganisms using a plurality of primer sets, each comprising a first and second primer, wherein the each primer set specifically recognizes the nucleotide sequence of a different microorganism, wherein the first primer of each primer set introduces a first binding sequence that is capable of binding to a capture DNA binding domain (DBD) and incapable of binding to any of a plurality of detection DBDs, and the second primer of each primer set introduces a second binding sequence that is capable of binding to one of the plurality of detection DBDs, wherein the first binding sequence is the same for each primer set and the second binding sequence is unique to each primer set; (b) binding the first binding sequence of the amplified polynucleotide to the capture DBD, wherein the capture DBD is affixed to a scaffold; (c) binding the second binding sequence of the amplified polynucleotide to a plurality of detection DBDs, wherein the detection DBDs are each attached to a detectable label; and (d) detecting the label.

In some embodiments, each detectable label is unique to the detection DBD, and the microorganism is detected by detecting which detectable label is present. In other embodiments, each detectable label is the same, wherein the amplified polynucleotide is sequentially contacted with each of the plurality of detection DBDs, wherein a detection step is performed between each sequential contacting step, and wherein the microorganism is detected by the sequential contacting step in which the detectable label is detected.

In some embodiments, the method further comprises the step of washing the amplified polynucleotide bound to the capture DBD prior to binding the detection DBD. In other embodiments, the method further comprises the step of washing the amplified polynucleotide bound to the detection DBD prior to the detection step. In other embodiments, the method further comprises the step of washing the amplified polynucleotide bound to the capture DBD after each sequential detection step.

In some embodiments, the amplifying step is performed by isothermal amplification. Optionally, the amplifying step is performed by PCR amplification.

In some embodiments, at least one of the detectable labels is capable of being detected by a surface acoustic wave device. Optionally, at least one of the detectable labels is selected from the group of a charged particle, a magnetic particle, a metal particle and a spore. In other embodiments, at least one of the detectable labels is capable of being detected by a field effect transistor. Optionally, at least one of the detectable labels is selected from the group of a magnetic particle, a metal particle, and a lipid vesicle comprising a charged solution. Optionally, the charged solution is an ionic solution. In other embodiments, at least one of the detectable labels is selected from the group of a fluorescent label, an enzymatic label, and a radioactive label.

In some embodiments, the method is performed on a microfluidics device. In other embodiments, each of the plurality of detection DBDs is released from a separate channel in the microfluidics device. In other embodiments, the sample is obtained from a subject.

An eighth aspect of the present disclosure provides a microfluidics device for detecting a microorganism in a sample comprising: (a) means for receiving the sample; (b) means for amplifying a polynucleotide from the microorganism and introducing a first binding site that is capable of binding to a capture DNA binding domain (DBD) and is incapable of binding to a detection DBD and a second binding site that is capable of binding to the detection DBD and is incapable of binding to the capture DBD; (c) the capture DBD affixed to a scaffold; (d) means for contacting the amplified polynucleotide with the capture DBD; (e) the detection DBD attached to a detectable label; (f) means for contacting the amplified polynucleotide with the detection DBD; and (g) means for detecting the label.

In some embodiments, the released polynucleotide is amplified by isothermal amplification. Optionally, the released polynucleotide is amplified by PCR amplification.

In some embodiments, the means for detecting the detectable label is a surface acoustic wave device. Optionally, the detectable label is selected from the group of a charged particle, a magnetic particle, a metal particle and a spore. In other embodiments, the means for detecting the detectable label is a field effect transistor. Optionally, the detectable label is selected from the group of a magnetic particle, a metal particle and a lipid vesicle comprising a charged solution. Optionally, the charged solution is an ionic solution. In other embodiments, the means for detecting the detectable label is selected from the group of means for detecting a fluorescent label, means for detecting an enzymatic label, and means for detecting a radioactive label.

In some embodiments, the device further comprises means for washing the amplified polynucleotide bound to the capture DBD. In other embodiments, the device further comprises means for washing the amplified polynucleotide bound to the detection DBD.

In some embodiments, the device comprises means for cycling an electric field or a magnetic field.

A ninth aspect of the present disclosure provides a microfluidics device for detecting one of a plurality of microorganisms in a sample comprising: (a) means for receiving the sample; (b) means for amplifying a polynucleotide from a plurality of microorganisms and introducing (i) a capture binding site that is capable of binding a capture DNA binding domain (DBD) and is not capable of binding any of a plurality of detection DBDs and (ii) detection binding sites that are capable of binding a plurality of detection DBDs and are incapable of binding the capture DBD; (c) the capture DBD affixed to a scaffold; (d) means for contacting the amplified polynucleotide with the capture DBD; (e) the plurality of detection DBDs, wherein each detection DBD is attached to a detectable label; (f) means for contacting the amplified polynucleotide with the plurality of detection DBDs; and (g) means for detecting the detectable label attached to each of the plurality of detection DBDs.

In some embodiments, the released polynucleotide is amplified by isothermal amplification. Optionally, the released polynucleotide is amplified by PCR amplification.

In some embodiments, each detection DBD is conjugated to a different detectable signal. In other embodiments, each detection DBD is conjugated to the same detectable signal, wherein the amplified polynucleotide is sequentially contacted with each of the plurality of detection DBDs, wherein a detecting step is performed between each sequential contacting step, and wherein the microorganism is detected by in which sequential contacting step the detectable label is detected.

In some embodiments, the microfluidics device further comprises means for washing the amplified polynucleotide bound to the capture DBD. In other embodiments, the device comprises means for washing the amplified polynucleotide bound to the detection DBD. In other embodiments, the device comprises means for washing the amplified polynucleotide bound to the capture DBD after each sequential detecting step.

In some embodiments, at least one means for detecting the detectable label is a surface acoustic wave device. Optionally, the detectable label is selected from the group of a charged particle, a magnetic particle, a metal particle and a spore. In other embodiments, at least one means for detecting the detectable label is a field effect transistor. Optionally, the detectable label is selected from the group of a magnetic particle, a metal particle, and a lipid vesicle comprising a charged solution. Optionally, the charged solution is ionic solution. In other embodiments, at least one means for detecting the detectable label is selected from the group of means for detecting a fluorescent label, means for detecting an enzymatic label, and means for detecting a radioactive label.

In some embodiments, each of the plurality of the detection DBDs may be released from a separate channel in the microfluidics device.

Particular embodiments of the disclosure are set forth in the following numbered paragraphs:

1. An antibody conjugate comprising an antibody linked to a polynucleotide, wherein the polynucleotide comprises a first binding nucleotide sequence that is capable of binding to a capture DNA binding domain (DBD) but incapable of binding to a detection DBD and a second binding nucleotide sequence that is capable of binding to the detection DBD but incapable of binding to the capture DBD.
2. The antibody conjugate according to paragraph 1, wherein the capture DBD comprises a helix-turn-helix motif.
3. The antibody according to paragraph 2, wherein the capture DBD is selected from the group consisting of MAT α1, MAT α2, MAT a1, Antennapedia, Ultrabithorax, Engrailed, Paired, Fushi tarazu, HOX, Unc86, Oct1, Oct2, Oct4, hRFX1, Pit, TCF-1, SRY, TrpR, RuvC, LexA, Lac I repressor, Bacteriophage Lambda Cro repressor, Bacteriophage 434 C1 and Cro repressors, P22 C2 repressor, and Bacteriophage Mu Ner protein, and variants thereof.
4. The antibody conjugate according to paragraph 1, wherein the capture DBD comprises a zinc-finger motif.
5. The antibody according to paragraph 4, wherein the capture DBD is selected from the group consisting of Zif268, SWI5, SIP1, FOG, Msn2p, A20, Klf4, Mac1, steroid receptors, the yeast transcriptional activator GAL4, and Krüppel and Hunchback and variants thereof.
6. The antibody conjugate according to paragraph 1, wherein the capture DBD comprises a basic leucine zipper motif.
7. The antibody according to paragraph 6, wherein the capture DBD is selected from the group consisting of, c-Fos/c-Jun, AP-1 Fos/Jun, CREB, ATF1, ATF2, ATF4, ATF5, ATF6, ATF7, BACH1, BACH2, BATF, BATF2, CEBPA, CEBPB, CEBPD, CEBPE, CEBPG, CEBPZ, CREB1, CREB3, CREB3L1, CREB3L2, CREB3L3, CREB3L4, CREB5, CREBL1, CREM, E4BP4, FOSL1, FOSL2, GCN4, JUN, JUNB, JUND, MAFA, MAFB, NFE2, NFE2L2, NFE2L3, SNFT, XBP1 OPAQUE, NFE2L2, and Bzip Maf and variants thereof.
8. The antibody conjugate according to paragraph 1, wherein the capture DBD comprises a helix-loop-helix motif.
9. The antibody according to paragraph 8, wherein the capture DBD is selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, BHLHE41, CLOCK, BMAL-1-CLOCK, EPAS1, FERD3L, FIGLA, HAND1, HAND2, HES1, HES2, HES3, HES4, HES5, HES6, HES7, HEY1, HEY2, HIF, HIF1A, ICE1, ID1, ID2, ID3, ID4, KIAA2018, LYL1, MASH1, MATH2, MAX, MESP1, MESP2, MIST1, MITF, MLX, MLXIP, MLXIPL, MNT, MOP5, MSC, MSGN1, MXD1, MXD3, MXD4, MXI1, C-Myc, N-Myc, MyoD, MYC, MYCL1, MYCL2, MYCN, MYF5, MYF6, MYOD1, MYOG, NCOA1, NCOA3, Neurogenins, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, Pho4, PTF1A, SCL, Scleraxis, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, USF2, Beta2/NeuroD1, Daughterless, Achaete-scute (T3), E12 and E47 and variants thereof.
10. The antibody conjugate according to any one of paragraphs 1-9, wherein the detection DBD comprises a helix-turn-helix motif.
11. The antibody according to paragraph 10, wherein the detection DBD is selected from the group consisting of MAT α1, MAT α2, MAT a1, Antennapedia, Ultrabithorax, Engrailed, Paired, Fushi tarazu, HOX, Unc86, Oct1, Oct2, Oct4, hRFX1, Pit, TCF-1, SRY, TrpR, RuvC, LexA, Lac I repressor, Bacteriophage Lambda Cro repressor, Bacteriophage 434 C1 and Cro repressors, P22 C2 repressor, and Bacteriophage Mu Ner protein, and variants thereof.
12. The antibody conjugate according to any one of paragraphs 1-19, wherein the detection DBD comprises a zinc-finger motif.
13. The antibody according to paragraph 12, wherein the detection DBD is selected from the group consisting of Zif268, SWI5, SIP1, FOG, Msn2p, A20, Klf4, Mac1, steroid receptors, the yeast transcriptional activator GAL4, and Krüppel and Hunchback and variants thereof.
14. The antibody conjugate according to any one of paragraphs 1-9, wherein the detection DBD comprises a basic leucine zipper motif.
15. The antibody according to paragraph 14, wherein the detection DBD is selected from the group consisting of c-Fos/c-Jun, AP-1 Fos/Jun, CREB, ATF1, ATF2, ATF4, ATF5, ATF6, ATF7, BACH1, BACH2, BATF, BATF2, CEBPA, CEBPB, CEBPD, CEBPE, CEBPG, CEBPZ, CREB1, CREB3, CREB3L1, CREB3L2, CREB3L3, CREB3L4, CREB5, CREBL1, CREM, E4BP4, FOSL1, FOSL2, GCN4, JUN, JUNB, JUND, MAFA, MAFB, NFE2, NFE2L2, NFE2L3, SNFT, XBP1 OPAQUE, NFE2L2, and Bzip Maf, and variants thereof.
16. The antibody conjugate according to any one of paragraphs 1-9, wherein the detection DBD comprises a helix-loop-helix motif.
17. The antibody according to paragraph 16, wherein the detection DBD is selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, BHLHE41, CLOCK, BMAL-1-CLOCK, EPAS1, FERD3L, FIGLA, HAND1, HAND2, HES1, HES2, HES3, HES4, HES5, HES6, HES7, HEY1, HEY2, HIF, HIF1A, ICE1, ID1, ID2, ID3, ID4, KIAA2018, LYL1, MASH1, MATH2, MAX, MESP1, MESP2, MIST1, MITF, MLX, MLXIP, MLXIPL, MNT, MOP5, MSC, MSGN1, MXD1, MXD3, MXD4, MXI1, C-Myc, N-Myc, MyoD, MYC, MYCL1, MYCL2, MYCN, MYF5, MYF6, MYOD1, MYOG, NCOA1, NCOA3, Neurogenins, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, Pho4, PTF1A, SCL, Scleraxis, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, USF2, Beta2/NeuroD1, Daughterless, Achaete-scute (T3), E12 and E47, and variants thereof.
18. The antibody conjugate according to any one of paragraphs 1-17, wherein the first binding nucleotide sequence is separated from the second binding nucleotide sequence by a linker sufficient to avoid steric hindrance between the capture DBD and the detection DBD.
19. The antibody conjugate according to paragraph 18, wherein the linker is in the range of 10 to 50 nucleotides long.
20. The antibody conjugate according to any one of paragraphs 1-19, wherein the polynucleotide further comprises a first amplification nucleotide sequence and a second amplification nucleotide sequence, wherein the first binding nucleotide sequence and the second binding nucleotide sequence are between the first amplification nucleotide sequence and the second amplification nucleotide sequence.
21. The antibody conjugate according to paragraph 20, wherein the first amplification nucleotide sequence and the second amplification nucleotide sequence are suitable for isothermal amplification.
22. The antibody conjugate according to paragraph 20, wherein the first amplification nucleotide sequence and the second amplification nucleotide sequence are suitable for PCR amplification.
23. The antibody conjugate according to any one of paragraphs 1-22, wherein the polynucleotide is capable of being released from the antibody upon antigen binding.
23. The antibody conjugate according to paragraph 22, wherein the polynucleotide is released by cleavage of a disulfide bond.
24. The antibody conjugate according to any one of paragraphs 1-23, wherein the antibody is linked to the polynucleotide through a protein.
25. The antibody conjugate according to paragraph 24, wherein the protein is selected from the group consisting of protein A, protein G, and protein L.
26. A method for detecting a marker in a sample, the method comprising:
(a) contacting the sample with the antibody conjugate according to any one of paragraphs 1-25, wherein the antibody portion of the conjugate binds to the marker;
(b) amplifying the polynucleotide portion of the antibody conjugate;
(c) binding the first nucleotide binding sequence of the amplified polynucleotide to a capture DNA binding domain (DBD), wherein the capture DBD is affixed to a scaffold;
(d) binding the second nucleotide binding sequence of the amplified polynucleotide to a detection DBD, wherein the detection DBD is affixed to a detectable label; and
(e) detecting the detectable label.
27. The method according to paragraph 26, wherein the marker is a biomarker, an environmental marker, an allergen, or a microorganism.
28. The method according to paragraph 27, wherein the microorganism is selected from the group consisting of a bacterium, a fungus, an archaeon, an alga, a protozoan and a virus.
29. The method according to any one of paragraphs 26-28, wherein the sample is an environmental sample, a food sample, or a sample obtained from a subject.
30. The method according to paragraph 26, wherein the polynucleotide is released upon binding of the marker to the antibody conjugate.
31. The method according to paragraph 26, wherein the marker is bound to a capture molecule, wherein the capture molecule is affixed to a scaffold or capable of being affixed to a scaffold.
32. The method according to paragraph 31, wherein the method further comprises the step of washing the capture molecule-bound marker prior to contacting the sample with the antibody conjugate.
33. The method according to any of paragraphs 31 or 32, wherein the method further comprises the step of washing the antibody conjugate-bound marker before the amplification step.
34. The method according to any one of paragraphs 26-33, further comprising the step of washing the amplified polynucleotide bound to the capture DBD prior to binding the detection DBD.
35. The method according to any one of paragraphs 26-34, further comprising the step of washing the amplified polynucleotide bound to the detection DBD prior to detecting the detectable label.
36. The method according to any one of paragraphs 26-34, wherein the amplifying step is performed by isothermal amplification.
37. The method according to any one of paragraphs 26-36, wherein the amplifying step is performed by PCR amplification.
38. The method according to paragraph 36 or 37, wherein the amplification comprises the step of binding a first amplification primer to a first amplification sequence in the polynucleotide portion of the antibody conjugate and binding a second amplification primer to a second amplification sequence in the polynucleotide portion of the antibody conjugate.
39. The method according to any one of paragraphs 26-38, wherein the detectable label is selected from the group consisting of a fluorescent label, a fluorogenic label, a dye, a colorimetric label, a magnetic label, a radioactive label, a luminescent label, a chemiluminescent label, and an enzymatic label.
40. The method according to any one of paragraphs 26-38, wherein the detectable label is capable of being detected by a surface acoustic wave device.
41. The method according to paragraph 40, wherein the detectable label is selected from the group consisting of a charged particle, a magnetic particle, a metal particle, a particle of 1 pg or greater, and a spore or a combination thereof.
42. The method according to any one of paragraphs 26-38, wherein the detectable label is capable of being detected by a field effect transistor.
43. The method according to paragraph 42, wherein the detectable label is selected from the group consisting of a charged particle, a magnetic particle, a metal particle, and an ionic solution or a combination thereof.
44. The method according to paragraph 43, wherein the ionic solution is comprised within a lipid vesicle, and the method comprises releasing the ionic solution from the lipid vesicle.
45. The method according to paragraph 44, wherein the ionic solution comprises a metal ion.
46. The method according to paragraph 45, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
47. The method according to paragraph 45, further comprising contacting the metal ion in the released ionic solution with a metal ion chelator or metal ion derivatized chelator, wherein the metal ion chelator or metal ion derivatized chelator is located at or near the detector.
48. The method according to paragraph 46, wherein the metal ion is Ca²⁺.
49. The method according to paragraph 48, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
50. The method according to any one of paragraphs 26-49, wherein the method is performed on a microfluidics device.
51. The method according to any one of paragraphs 26-50, wherein the marker is present in the sample at a concentration that cannot be detected without signal amplification.
52. A method of detecting one of a plurality of markers in a sample, the method comprising:
(a) contacting the sample with a first antibody conjugate and a second antibody conjugate, wherein the first antibody conjugate and second antibody conjugate are antibody conjugates according to any one of paragraphs 1-25, wherein the antibody portion of the first antibody conjugate binds a different marker than the antibody portion of the second antibody conjugate, wherein the first binding nucleotide sequence of the polynucleotide portion of the first antibody conjugate binds to a first capture DNA binding domain (DBD) and the first binding nucleotide sequence of the polynucleotide portion of the second antibody conjugate binds to a second capture DBD, and wherein the second binding nucleotide sequence of the polynucleotide portion of the first antibody conjugate binds to a first detection DBD, and the second binding nucleotide sequence of the polynucleotide portion of the second antibody conjugate binds to the second detection DBD;
(b) amplifying the polynucleotide portion of the marker-bound antibody conjugate;
(c) binding the first nucleotide binding sequence of the amplified polynucleotide to a capture DBD, wherein the capture DBD binds to the first binding nucleotide sequence of the first and second antibody conjugates and is affixed to a scaffold;
(d) binding the second binding nucleotide sequence of the amplified polynucleotide to a first detection DBD, wherein the first detection DBD binds to the second binding nucleotide sequence of the first antibody conjugate and is affixed to a first detectable label;
(e) performing a first detection step to detect the first detectable label;
(f) binding the amplified polynucleotide to a second detection DBD, wherein the second detection DBD binds to the second binding nucleotide sequence of the second antibody conjugate and is affixed to a second detectable label; and
(g) performing a second detection step to detect the second detectable label.
53. The method according to paragraph 52, wherein the first and second capture DBDs bind the same first binding nucleotide sequence.
54. The method according to paragraph 52, wherein the first and second capture DBDs are the same DBD.
55. The method according to paragraph 52, wherein the first and second capture DBDs bind different first binding nucleotide sequences.
56. The method according to any one of paragraphs 52-55, wherein the polynucleotide portion of each antibody conjugate is released upon binding of the marker to the antibody conjugate.
57. The method according to paragraph 52, wherein each of the plurality of markers is bound to a capture molecule, wherein each capture molecule is affixed to a scaffold or capable of being affixed to a scaffold.
58. The method according to paragraph 57, wherein the method further comprises the step of washing the capture molecule-bound marker prior to contacting the sample with the antibody conjugates.
59. The method according to paragraph 58, wherein the method further comprises the step of washing the antibody conjugate-bound markers before the amplification step.
60. The method according to any one of paragraphs 52-59, wherein the first detectable label and the second detectable label are different, and the marker is detected by which detectable label is present.
61. The method according to any one of paragraphs 52-60, wherein the first detectable label and the second detectable label are the same, and the marker is detected by whether the detectable label is present in the first detection step or the second detection step.
62. The method according to any one of paragraphs 52-61, further comprising the step of washing the amplified polynucleotide bound to the capture DBD prior to binding the detection DBD.
63. The method according to any one of paragraphs 52-62, further comprising the step of washing the amplified polynucleotide bound to the first detection DBD prior to first detection step.
64. The method according to any one of paragraphs 52-63, further comprising the step of washing the amplified polynucleotide bound to the capture DBD after the first detection step prior to binding the second detection DBD.
65. The method according to any one of paragraphs 52-64, further comprising the step of washing the amplified polynucleotide bound to the second detection DBD prior to second detection step.
66. The method according to any one of paragraphs 52-65, wherein the amplifying step is performed by isothermal amplification.
67. The method according to any one of paragraphs 52-66, wherein the amplifying step is performed by PCR amplification.
68. The method according to paragraph 66 or 67, wherein the amplification comprises the step of binding a first amplification primer to a first amplification sequence in the polynucleotide portion of the antibody conjugate and binding a second amplification primer to a second amplification sequence in the polynucleotide portion of the antibody conjugate.
69. The method according to paragraph 68, wherein the first amplification sequence in the polynucleotide portion of the first antibody conjugate is the same as the first amplification sequence in the polynucleotide portion of the second antibody conjugate.
70. The method according to paragraph 68 or 69, wherein the second amplification sequence in the polynucleotide portion of the first antibody conjugate is the same as the second amplification sequence in the polynucleotide portion of the second antibody conjugate.
71. The method according to any one of paragraphs 52-70, wherein the first detectable label is selected from the group consisting of a fluorescent label, an enzymatic label, a fluorogenic label, a dye, a colorimetric label, a magnetic label, a luminescent label, a chemiluminescent label and a radioactive label.
72. The method according to any one of paragraphs 52-70, wherein the first detectable label is capable of being detected by a surface acoustic wave device.
73. The method according to paragraph 72, wherein the first detectable label is selected from the group consisting of a magnetic particle, a metal particle, a particle of 1 pg or greater, a charged particle and a spore or a combination thereof.
74. The method according to any one of paragraphs 52-70, wherein the first detectable label is capable of being detected by a field effect transistor.
75. The method according to paragraph 74, wherein the first detectable label is selected from the group consisting of a magnetic particle, a metal particle, a charged particle and an ionic solution or a combination thereof.
76. The method according to paragraph 75, wherein the ionic solution is comprised within a lipid vesicle, and the method comprises releasing the ionic solution from the lipid vesicle.
77. The method according to paragraph 76, wherein the ionic solution comprises a metal ion.
78. The method according to paragraph 77, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
79. The method according to paragraph 77, further comprising contacting the metal ion in the released ionic solution with a metal ion chelator or metal ion derivatized chelator, wherein the metal ion chelator or metal ion derivatized chelator is located at or near the detector.
80. The method according to paragraph 79, wherein the metal ion is Ca²⁺.
81. The method according to paragraph 80, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
82. The method according to any one of paragraphs 52-81, wherein the second detectable label is capable of being detected by a surface acoustic wave device.
83. The method according to paragraph 82, wherein the second detectable label is selected from the group consisting of a magnetic particle, a metal particle, a particle of 1 pg or greater, a charged particle and a spore or a combination thereof.
84. The method according to any one of paragraphs 52-81, wherein the second detectable label is capable of being detected by a field effect transistor.
85. The method according to paragraph 84, wherein the second detectable label is selected from the group consisting of a charged particle, magnetic particle, a metal particle and an ionic solution or a combination thereof.
86. The method according to paragraph 85, wherein the ionic solution is comprised within a lipid vesicle, and the method comprises releasing the ionic solution from the lipid vesicle.
87. The method according to paragraph 86, wherein the ionic solution comprises a metal ion.
88. The method according to paragraph 87, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
89. The method according to paragraph 87, further comprising contacting the metal ion in the released ionic solution with a metal ion chelator or metal ion derivatized chelator, wherein the metal ion chelator or metal ion derivatized chelator is located at or near the detector.
90. The method according to paragraph 89, wherein the metal ion is Ca²⁺.
91. The method according to paragraph 78, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
92. The method according to any one of paragraphs 52-71, wherein the second detectable label is selected from the group consisting of a fluorescent label, an enzymatic label, a fluorogenic label, a dye, a colorimetric label, a magnetic label, a luminescent label, a chemiluminescent label and a radioactive label.
93. The method according to any one of paragraphs 52-92, wherein the method is performed on a microfluidics device.
94. The method according to paragraph 93, wherein the first detection DBD is released from a first channel in the microfluidics device.
95. The method according to paragraph 93 or 94, wherein the second detection DBD is released from a second channel in the microfluidics device.
96. The method according to paragraph 95, wherein the release of the first detection DBD from the first channel and the first detection step occur before the release of the second detection DBD from the second channel.
97. The method according to any one of paragraphs 52-96, wherein the method further comprises:
(1) contacting the sample with one or more additional antibody conjugates, wherein each of the one or more additional antibody conjugates is an antibody conjugate according to any one of paragraphs 1-25, wherein each of the antibody portion of the one or more additional antibody conjugates binds a different marker than the antibody portion of the first antibody conjugate, the antibody portion of the second antibody conjugate and the antibody portion of the any other additional antibody conjugates; wherein the first binding nucleotide sequence of each of the one or more additional antibody conjugates binds to the same or different capture DBD as the first binding nucleotide sequence of the first and second antibody conjugates, wherein the second nucleotide binding sequence of each of the one or more additional antibody conjugates binds to the same or a different DBD than the second nucleotide binding sequence of the first antibody conjugate, the second antibody conjugate and any other additional antibody conjugates;
(2) binding the amplified polynucleotide to one or more additional DBDs, wherein each of the one or more additional DBDs binds to the second binding nucleotide sequence of the one or more additional antibody conjugates and is affixed to a one or more additional detectable label;
(3) performing one or more additional detection steps to detect the one or more additional detectable labels.
98. The method according to any one of paragraphs 52-97, wherein the marker is present in the sample at a concentration that cannot be detected without signal amplification.
99. A microfluidics device comprising:
(a) means for receiving a sample;
(b) an antibody conjugate, wherein the conjugate comprises an antibody linked to a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD;
(c) means for contacting the sample with the antibody conjugate;
(d) means for amplifying the polynucleotide portion of the antibody conjugate;
(e) the capture DBD affixed to a scaffold;
(f) means for contacting the amplified polynucleotide with the capture DBD;
(g) the detection DBD attached to a detectable label, wherein the capture DBD and the detection DBD are different;
(h) means for contacting the amplified polynucleotide with the detection DBD; and
(i) means for detecting the detectable label.
100. The microfluidics device according to paragraph 99, wherein the sample is an environmental sample, a food sample, or a sample obtained from a subject.
101. The microfluidics device according to paragraphs 99 or 100, wherein the antibody conjugate is an antibody conjugate according to any one of paragraphs 1-25.
102. The microfluidics device according to any one of paragraphs 99-101, wherein the polynucleotide portion of the antibody conjugate is amplified by isothermal amplification.
103. The microfluidics device according to any one of paragraphs 99-101, wherein the polynucleotide portion of the antibody conjugate is amplified by PCR.
104. The microfluidics device according to paragraphs 102 or 103, wherein the microfluidics device further comprises a first amplification primer that binds to a first amplification sequence in the polynucleotide portion of the antibody conjugate and a second amplification primer that binds to a second amplification sequence in the polynucleotide portion of the antibody conjugate.
105. The microfluidics device according to any one of paragraphs 99-104, wherein the means for detecting the detectable label is a surface acoustic wave device.
106. The microfluidics device according to paragraph 105, wherein the detectable label is selected from the group consisting of a charged particle, magnetic particle, a metal particle, a particle of 1 pg or greater, and a spore or a combination thereof.
107. The microfluidics device according to any one of paragraphs 99-104, wherein the means for detecting the detectable label is a field effect transistor.
108. The microfluidics device according to paragraph 107, wherein the detectable label is selected from the group consisting of a magnetic particle, a metal particle, and an ionic solution or a combination thereof.
109. The microfluidics device according to paragraph 108, wherein the ionic solution is comprised within a lipid vesicle, and the microfluidics comprises means for releasing the ionic solution from the lipid vesicle.
110. The microfluidics device according to paragraph 109, wherein the ionic solution comprises a metal ion.
111. The microfluidics device according to paragraph 110, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
112. The microfluidics device according to paragraph 110, further comprising a metal ion chelator or metal ion derivatized chelator specific for the metal ion in the ionic solution, wherein the metal ion chelator or metal ion derivatized chelator is located at or near the detector.
113. The microfluidics device according to paragraph 112, wherein the metal ion is Ca²⁺.
114. The microfluidics device according to paragraph 113, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
115. The microfluidics device according to any one of paragraphs 109-114, wherein the means for releasing the ionic solution from the lipid vesicle comprises a detergent.
116. The microfluidics device according to paragraph 115, wherein the detergent is a non-ionic detergent.
117. The microfluidics device according to any one of paragraphs 99-104, wherein the means for detecting the detectable label is selected from the group consisting of means for detecting a fluorescent label, means for detecting an enzymatic label, means for detecting a fluorogenic label, means for detecting a dye, means for detecting a colorimetric label, means for detecting a magnetic label, means for detecting a luminescent label, means for detecting a chemiluminescent label and means for detecting a radioactive label.
118. The microfluidics device according to any one of paragraphs 99-117, wherein the device further comprises means for washing the amplified polynucleotide bound to the capture DBD.
119. The microfluidics device according to any one of paragraphs 99-118, wherein the device further comprises means for washing the amplified polynucleotide bound to the detection DBD.
120. The microfluidics device according to any one of paragraphs 99-119, wherein the device further comprises means for immobilizing the marker.
121. The microfluidics device according to paragraph 120, wherein the means for immobilizing the marker is an antibody that binds the marker and is affixed to a solid phase or is capable of being affixed to a solid phase.
122. The microfluidics device according to paragraph 120 or 121, wherein the device further comprises means for washing the immobilized marker.
123. The microfluidics device according to any one of paragraphs 99-122, wherein the device comprises means for cycling an electric field or a magnetic field.
124. A microfluidics device comprising:
(a) means for receiving a sample;
(b) a first antibody conjugate, wherein the first antibody conjugate comprises a first antibody linked to a first polynucleotide, wherein the first polynucleotide comprises a capture nucleotide sequence that is capable of binding to a capture DNA binding domain (DBD) and is incapable of binding to a first or second detection DBD and a first detection nucleotide sequence that is capable of binding to the first detection DBD and is incapable of binding to the capture DBD or the second detection DBD;
(c) a second antibody conjugate, wherein the second antibody conjugate comprises a second antibody linked to a second polynucleotide, wherein the second polynucleotide comprises a capture nucleotide sequence that is capable of binding to the capture DBD and is incapable of binding to the first or second detection DBD and a second detection nucleotide sequence that is capable of binding to the second detection DBD and is incapable of binding to the capture DBD or the first detection DBD;
(d) means for contacting the sample with the antibody conjugates;
(e) means for amplifying the polynucleotide portions of the first and second antibody conjugates;
(f) the capture DBD affixed to a scaffold;
(g) means for contacting the amplified polynucleotides with the capture DBD;
(h) the first detection DBD attached to a first detectable label, the second detection DBD attached to a second detectable label;
(i) means for contacting the amplified polynucleotides with the first and second detection DBDs; and
(j) means for detecting the first and second detectable labels.
125. The microfluidics device according to paragraph 124, wherein the first antibody conjugate is an antibody conjugate according to any one of paragraphs 1-25.
126. The microfluidics device according to paragraphs 124 or 125, wherein the second antibody conjugate is an antibody conjugate according to any one of paragraphs 1-25.
127. The microfluidics device according to any one of paragraphs 124-126, wherein the polynucleotide portions of the antibody conjugates are amplified by isothermal amplification.
128. The microfluidics device according to any one of paragraphs 124-126, wherein the polynucleotide portions of the antibody conjugates are amplified by PCR.
129. The microfluidics device according to paragraphs any one of paragraphs 124-128, wherein the microfluidics device further comprises a first amplification primer that binds to a first amplification sequence in the polynucleotide portion of the first or second antibody conjugate and a second amplification primer that binds to a second amplification sequence in the polynucleotide portion of the first or second antibody conjugate.
130. The microfluidics device according to any one of paragraphs 124-129, wherein the first detectable signal and the second detectable signal are different.
131. The microfluidics device according to any one of paragraphs 124-129, wherein the first detectable signal and the second detectable signal are the same.
132. The microfluidics device according to any one of paragraphs 124-131, wherein the first detection DBD is stored in a first channel and the second detection DBD is stored in a second channel.
133. The microfluidics device according to paragraph 132, wherein the first detection DBD is released from the first channel and the means for detecting the first detectable label is performed prior to the release of the second detection DBD from the second channel.
134. The microfluidics device according to any one of paragraphs 124-133, wherein the means for detecting the first detectable label is a surface acoustic wave device.
135. The microfluidics device according to paragraph 134, wherein the first detectable label is selected from the group consisting of a charged particle, a magnetic particle, a metal particle, a particle of 1 pg or greater, and a spore or a combination thereof.
136. The microfluidics device according to any one of paragraphs 124-133, wherein the means for detecting the first detectable label is a field effect transistor.
137. The microfluidics device according to paragraph 136, wherein the first detectable label is selected from the group consisting of a charged particle, a magnetic particle, a metal particle, and an ionic solution or a combination thereof.
138. The microfluidics device according to paragraph 137, wherein the ionic solution is comprised within a lipid vesicle, and the microfluidics comprises means for releasing the ionic solution from the lipid vesicle.
139. The microfluidics device according to paragraph 138, wherein the ionic solution comprises a metal ion.
140. The microfluidics device according to paragraph 139, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
141. The microfluidics device according to paragraph 139, further comprising a metal ion chelator or metal ion derivatized chelator specific for the metal ion in the ionic solution, wherein the metal ion chelator or metal ion derivatized chelator is located at or near the detector.
142. The microfluidics device according to paragraph 141, wherein the metal ion is Ca²⁺.
143. The microfluidics device according to paragraph 142, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
144. The microfluidics device according to any one of paragraphs 138-143, wherein the means for releasing the ionic solution from the lipid vesicle comprises a detergent.
145. The microfluidics device according to paragraph 144, wherein the detergent is a non-ionic detergent.
146. The microfluidics device according to any one of paragraphs 124-133, wherein the means for detecting the first detectable label is selected from the group consisting of means for detecting a fluorescent label, means for detecting an enzymatic label, means for detecting a fluorogenic label, means for detecting a dye, means for detecting a colorimetric label, means for detecting a magnetic label, means for detecting a luminescent label, means for detecting a chemiluminescent label and means for detecting a radioactive label.
147. The microfluidics device according to any one of paragraphs 124-146, wherein the means for detecting the second detectable label is a surface acoustic wave device.
148. The microfluidics device according to paragraph 147, wherein the second detectable label is selected from the group consisting of a charged particle, a magnetic particle, a metal particle, a particle of 1 pg or greater, and a spore or a combination thereof.
149. The microfluidics device according to any one of paragraphs 124-146, wherein the means for detecting the second detectable label is a field effect transistor.
150. The microfluidics device according to paragraph 149, wherein the second detectable label is selected from the group consisting of a magnetic particle, a metal particle, and an ionic solution or a combination thereof.
151. The microfluidics device according to paragraph 150, wherein the ionic solution is comprised within a lipid vesicle, and the microfluidics comprises means for releasing the ionic solution from the lipid vesicle.
152. The microfluidics device according to paragraph 151, wherein the ionic solution comprises a metal ion.
153. The microfluidics device according to paragraph 152, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
154. The microfluidics device according to paragraph 152, further comprising a metal ion chelator or metal ion derivatized chelator specific for the metal ion in the ionic solution, wherein the metal ion chelator or metal ion derivatized chelator is located at or near the detector.
155. The microfluidics device according to paragraph 154, wherein the metal ion is Ca²⁺.
156. The microfluidics device according to paragraph 155, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
157. The microfluidics device according to any one of paragraphs 151-156, wherein the means for releasing the ionic solution from the lipid vesicle comprises a detergent.
158. The microfluidics device according to paragraph 157, wherein the detergent is a non-ionic detergent.
159. The microfluidics device according to any one of paragraphs 124-146, wherein the means for detecting the second detectable label is selected from the group consisting of means for detecting a fluorescent label, means for detecting an enzymatic label, means for detecting a fluorogenic label, means for detecting a dye, means for detecting a colorimetric label, means for detecting a magnetic label, means for detecting a luminescent label, means for detecting a chemiluminescent label and means for detecting a radioactive label.
160. The microfluidics device according to any one of paragraphs 124-146, wherein the microfluidics device further comprises one or more additional detection DBDs attached to one or more additional detectable labels.
161. The microfluidics device according to paragraph 160, wherein the one or more additional detectable labels are the same as the first and second detectable labels.
162. The microfluidics device according to paragraph 160, wherein each of the one or more additional detectable labels is different from the first detectable label, the second detectable label, and any other additional detectable labels.
163. The microfluidics device according to any one of paragraphs 160-162, wherein the microfluidics device comprises one or more additional antibody conjugates, wherein each of the one or more additional antibody conjugates comprises an antibody linked to one or more additional polynucleotides, wherein each of the one or more additional polynucleotides comprises a capture nucleotide sequence that binds to the capture DBD and does not bind to the first, second, or any additional detection DBD, and an additional detection nucleotide sequence that binds to the one or more additional detection DBDs and does not bind to the capture DBD, the first, second or any other additional detection DBD.
164. The microfluidics device of any one of paragraphs 124-163, wherein the device further comprises means for washing the amplified polynucleotide bound to the capture DBD.
165. The microfluidics device according to any one of paragraphs 124-164, wherein the device further comprises means for washing the amplified polynucleotide bound to the first detection DBD.
166. The microfluidics device according to any one of paragraphs 124-165, wherein the device further comprises means for washing the amplified polynucleotide bound to the second detection DBD.
167. The microfluidics device according to any one of paragraphs 124-166, wherein the device further comprises means for washing the amplified polynucleotide bound to the one or more additional detection DBDs.
168. The microfluidics device according to any one of paragraphs 124-167, wherein the device further comprises means for immobilizing the marker.
169. The microfluidics device according to paragraph 168, wherein the means for immobilizing the marker is an antibody that binds the marker and is affixed to a solid surface or is capable of being affixed to a solid surface.
170. The microfluidics device according to paragraphs 168 or 169, wherein the device further comprises means for washing the immobilized marker.
171. The microfluidics device according to any one of paragraphs 124-170, wherein the device comprises means for cycling an electric field or a magnetic field.
172. A method for detecting a microorganism in a sample comprising:
(a) amplifying a polynucleotide from the microorganism with a first and second primer, wherein the first primer introduces a first binding sequence capable of binding to a capture DNA binding domain (DBD) and incapable of binding to a detection DBD and the second primer introduces a second binding sequence capable of binding the detection DBD and incapable of binding the capture DBD;
(b) binding the first binding sequence of the amplified polynucleotide to the capture DBD, wherein the capture DBD is affixed to a scaffold;
(c) binding the second binding sequence of the amplified polynucleotide to the detection DBD, wherein the detection DBD is attached to a detectable label; and
(d) detecting the label.
173. The method according to paragraph 172, further comprising releasing the polynucleotide from the microorganism.
174. The method according to paragraphs 172 or 173, wherein the sample is an environmental sample, a food sample, or a sample obtained from a subject.
175. The method according to paragraph 172, wherein the microorganism is selected from the group consisting of a bacterium, a fungus, an archaeon, an alga, a protozoan and a virus.
176. The method according to paragraphs 172 or 173, further comprising the step of washing the amplified polynucleotide bound to the capture DBD prior to binding the detection DBD.
177. The method according to paragraph 172 or 173, further comprising the step of washing the amplified polynucleotide bound to the detection DBD prior to detecting the presence of the detectable label.
178. The method according to any one of paragraphs 172-177, wherein the amplifying step is performed by isothermal amplification.
179. The method according to any one of paragraphs 172-177, wherein the amplifying step is performed by PCR amplification.
180. The method according to any one of paragraphs 172-179, wherein the detectable label is capable of being detected by a surface acoustic wave device.
181. The method according to paragraph 180, wherein the detectable label is selected from the group consisting of a charged particle, a magnetic particle, a metal particle, a particle of 1 pg or greater, and a spore or a combination thereof.
182. The method according to any one of paragraphs 172-179, wherein the detectable label is capable of being detected by a field effect transistor.
183. The method according to paragraph 182, wherein the detectable label is selected from the group consisting of a charged particle, a magnetic particle, a metal particle, and an ionic solution or a combination thereof.
184. The method according to paragraph 183, wherein the ionic solution is comprised within a lipid vesicle, and the method comprises releasing the ionic solution from the lipid vesicle.
185. The method according to paragraph 184, wherein the ionic solution comprises a metal ion.
186. The method according to paragraph 185, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
187. The method according to paragraph 185, further comprising contacting the metal ion from the released ionic solution with a metal ion chelator or metal ion derivatized chelator, wherein the metal ion chelator or metal ion derivatized chelator is located at or near the detector.
188. The method according to paragraph 187, wherein the metal ion is Ca²⁺.
189. The method according to paragraph 188, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
190. The method according to any one of paragraphs 172-179, wherein the detectable label is selected from the group consisting of a fluorescent label, an enzymatic label, a fluorogenic label, a dye, a colorimetric label, a magnetic label, a luminescent label, a chemiluminescent label and a radioactive label.
191. The method according to any one of paragraphs 172-184, wherein the method is performed on a microfluidics device.
192. The method according to any one of paragraphs 172-191, wherein the sample is obtained from a subject.
193. A method for detecting one of a plurality of microorganisms in a sample comprising:
(a) amplifying a polynucleotide from the plurality of microorganisms using a plurality of primer sets, each comprising a first and second primer, wherein the each primer set specifically recognizes the nucleotide sequence of a different microorganism, wherein the first primer of each primer set introduces a first binding sequence that is capable of binding to a capture DNA binding domain (DBD) and incapable of binding to any of a plurality of detection DBDs, and the second primer of each primer set introduces a second binding sequence that is capable of binding to one of the plurality of detection DBDs, wherein the first binding sequence is the same for each primer set and the second binding sequence is unique to each primer set;
(b) binding the first binding sequence of the amplified polynucleotide to the capture DBD, wherein the capture DBD is affixed to a scaffold;
(c) binding the second binding sequence of the amplified polynucleotide to a plurality of detection DBDs, wherein the detection DBDs are each attached to a detectable label; and
(d) detecting the label.
194. The method according to paragraph 193, further comprising releasing the polynucleotides from the microorganisms.
195. The method according to paragraphs 193 or 194, wherein each detectable label is unique to the detection DBD, and the microorganism is detected by detecting which detectable label is present.
196. The method according to paragraph 193 or 194, wherein each detectable label is the same, wherein the amplified polynucleotide is sequentially contacted with each of the plurality of detection DBDs, wherein a detection step is performed between each sequential contacting step, and wherein the microorganism is detected by the sequential contacting step in which the detectable label is detected.
197. The method according to any one of paragraphs 193-196, further comprising the step of washing the amplified polynucleotide bound to the capture DBD prior to binding the detection DBD.
198. The method according to any one of paragraphs 193-196, further comprising the step of washing the amplified polynucleotide bound to the detection DBD prior to the detection step.
199. The method according to any one of paragraphs 198, further comprising the step of washing the amplified polynucleotide bound to the capture DBD after each sequential detection step.
200. The method according to any one of paragraphs 193-199, wherein the amplifying step is performed by isothermal amplification.
201. The method according to any one of paragraphs 193-199, wherein the amplifying step is performed by PCR amplification.
202. The method according to any one of paragraphs 193-201, wherein at least one of the detectable labels is capable of being detected by a surface acoustic wave device.
203. The method according to paragraph 193, wherein at least one of the detectable labels is selected from the group consisting of a charged particle, a magnetic particle, a particle of 1 pg or greater, a metal particle, and a spore or a combination thereof.
204. The method according to any one of paragraphs 193-203, wherein at least one of the detectable labels is capable of being detected by a field effect transistor.
205. The method according to paragraph 204, wherein at least one of the detectable labels is selected from the group consisting of a magnetic particle, a metal particle, and an ionic solution or a combination thereof.
206. The method according to paragraph 205, wherein the ionic solution is comprised within a lipid vesicle, and the method comprises releasing the ionic solution from the lipid vesicle.
207. The method according to paragraph 206, wherein the ionic solution comprises a metal ion.
208. The method according to paragraph 207, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
209. The method according to paragraph 207, further comprising contacting the metal ion from the released ionic solution with a metal ion chelator or metal ion derivatized chelator, wherein the metal ion chelator or metal ion derivatized chelator is located at or near the detector.
210. The method according to paragraph 208, wherein the metal ion is Ca²⁺.
211. The method according to paragraph 210, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
212. The method according to any one of paragraphs 193-211, wherein at least one of the detectable labels is selected from the group consisting of a fluorescent label, an enzymatic label, a fluorogenic label, a dye, a colorimetric label, a magnetic label, a luminescent label, a chemiluminescent label and a radioactive label.
213. The method according to any one of paragraphs 193-212, wherein the method is performed on a microfluidics device.
214. The method according to paragraph 213, wherein the each of the plurality of detection DBDs is released from a separate channel in the microfluidics device.
215. The method according to any one of paragraphs 193-214, wherein the sample is obtained from a subject.
216. A microfluidics device for detecting a microorganism in a sample comprising:
(a) means for receiving the sample;
(b) means for amplifying a polynucleotide from the microorganism and introducing a first binding site that is capable of binding to a capture DNA binding domain (DBD) and is incapable of binding to a detection DBD and a second binding site that is capable of binding to the detection DBD and is incapable of binding to the capture DBD;
(c) the capture DBD affixed to a scaffold;
(d) means for contacting the amplified polynucleotide with the capture DBD;
(e) the detection DBD attached to a detectable label;
(f) means for contacting the amplified polynucleotide with the detection DBD; and
(g) means for detecting the label.
217. The microfluidics device according to paragraph 216, further comprising means for releasing the polynucleotide from the microorganism.
218. The microfluidics device according to paragraphs 216 or 217, wherein the polynucleotide is amplified by isothermal amplification.
219. The microfluidics device according to paragraphs 216 or 217, wherein the polynucleotide is amplified by PCR amplification.
220. The microfluidics device according to any one of paragraphs 216-219, wherein the means for detecting the detectable label is a surface acoustic wave device.
221. The microfluidics device according to paragraph 220, wherein the detectable label is selected from the group consisting of a charged particle, a magnetic particle, a metal particle, a particle of 1 pg or greater, and a spore or a combination thereof.
222. The microfluidics device according to any one of paragraphs 216-219, wherein the means for detecting the detectable label is a field effect transistor.
223. The microfluidics device according to paragraph 222, wherein the detectable label is selected from the group consisting of a magnetic particle, a metal particle, and an ionic solution or a combination thereof.
224. The microfluidics device according to paragraph 223, wherein the ionic solution is comprised within a lipid vesicle, and the microfluidics comprises means for releasing the ionic solution from the lipid vesicle.
225. The microfluidics device according to paragraph 224, wherein the ionic solution comprises a metal ion.
226. The microfluidics device according to paragraph 225, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
227. The microfluidics device according to paragraph 225, further comprising a metal ion chelator or metal ion derivatized chelator specific from the metal ion in the ionic solution, wherein the metal ion chelator or metal ion derivatized chelator is located at or near the detector.
228. The microfluidics device according to paragraph 226, wherein the metal ion is Ca²⁺.
229. The microfluidics device according to paragraph 228, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
230. The microfluidics device according to any one of paragraphs 224-229, wherein the means for releasing the ionic solution from the lipid vesicle comprises a detergent.
231. The microfluidics device according to paragraph 230, wherein the detergent is a non-ionic detergent.
232. The microfluidics device according to any one of paragraphs 216-219, wherein the means for detecting the detectable label is selected from the group consisting of means for detecting a fluorescent label, means for detecting an enzymatic label, means for detecting a fluorogenic label, means for detecting a dye, means for detecting a colorimetric label, means for detecting a magnetic label, means for detecting a luminescent label, means for detecting a chemiluminescent label and means for detecting a radioactive label.
233. The microfluidics device according to any one of paragraphs 216-232, wherein the device further comprises means for washing the amplified polynucleotide bound to the capture DBD.
234. The microfluidics device according to any one of paragraphs 216-233, wherein the device further comprises means for washing the amplified polynucleotide bound to the detection DBD.
235. The microfluidics device according to any one of paragraphs 216-234, wherein the device comprises means for cycling an electric field or a magnetic field.
236. A microfluidics device for detecting one of a plurality of microorganisms in a sample obtained from a subject comprising
(a) means for receiving the sample;
(b) means for amplifying a polynucleotide from a plurality of microorganisms and introducing (i) a capture binding site that is capable of binding a capture DNA binding domain (DBD) and is not capable of binding any of a plurality of detection DBDs and (ii) detection binding sites that are capable of binding a plurality of detection DBDs and are incapable of binding the capture DBD;
(c) the capture DBD affixed to a scaffold;
(d) means for contacting the amplified polynucleotide with the capture DBD;
(e) the plurality of detection DBDs, wherein each detection DBD is attached to a detectable label;
(f) means for contacting the amplified polynucleotide with the plurality of detection DBDs; and
(g) means for detecting the detectable label attached to each of the plurality of detection DBDs.
237. The microfluidics device according to paragraph 236, further comprising means for releasing the polynucleotide from the plurality of microorganisms.
238. The microfluidics device according to paragraphs 236 or 237, wherein each detection DBD is unique to a specific microorganism from the plurality of microorganisms.
239. The microfluidics device according to paragraphs 236 or 237, wherein the polynucleotide is amplified by isothermal amplification.
240. The microfluidics device according to paragraphs 236 or 237, wherein the polynucleotide is amplified by PCR amplification.
241. The microfluidics device according to any one of paragraphs 236-240, wherein each detection DBD is attached to a different detectable label.
242. The microfluidics device according to any one of paragraphs 236-240, wherein if each detection DBD is attached to the same detectable label, the amplified polynucleotide is sequentially contacted with each of the plurality of detection DBDs, wherein a detection step is performed between each sequential contacting step, and wherein the microorganism is detected by in which sequential contacting step the detectable label is detected.
243. The microfluidics device according to any one of paragraphs 236-242, further comprising means for washing the amplified polynucleotide bound to the capture DBD.
244. The microfluidics device according to any one of paragraphs 238-243, further comprising means for washing the amplified polynucleotide bound to the detection DBD.
245. The microfluidics device according to paragraph 242, further comprising means for washing the amplified polynucleotide bound to the capture DBD after each sequential detection step.
246. The microfluidics device according to any one of paragraphs 236-245, wherein at least one means for detecting the detectable label is a surface acoustic wave device.
247. The microfluidics device according to paragraph 246, wherein the detectable label is selected from the group consisting of a charged particle, a magnetic particle, a metal particle, a particle of 1 pg or greater, and a spore or a combination thereof.
248. The microfluidics device according to any one of paragraphs 236-247, wherein at least one means for detecting the detectable label is a field effect transistor.
249. The microfluidics device according to paragraph 248, wherein the detectable label is selected from the group consisting of a magnetic particle, a metal particle, and an ionic solution or a combination thereof.
250. The microfluidics device according to paragraph 249, wherein the ionic solution is comprised within a lipid vesicle, and the microfluidics comprises means for releasing the ionic solution from the lipid vesicle.
251. The microfluidics device according to paragraph 250, wherein the ionic solution comprises a metal ion.
252. The microfluidics device according to paragraph 251, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion.
253. The microfluidics device according to paragraph 251, further comprising a metal ion chelator or metal ion derivatized chelator specific for the metal ion in the ionic solution, wherein the metal ion chelator or metal ion derivatized chelator is located at or near the detector.
254. The microfluidics device according to paragraph 252, wherein the metal ion is Ca²⁺.
255. The microfluidics device according to paragraph 254, wherein the chelator or the derivatized chelator is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (tetrapotassium salt); DMNP-EDTA; INDO 1 pentapotassium salt; FURA-2 pentapotassium salt; FURA 2/AM; MAPTAM; FLUO 3 (pentaammonium salt); Tetraacetoxymethyl Bis(2-aminoethyl) Ether N,N,Nprime,Nprime-Tetraacetic Acid; and derivatives thereof.
256. The microfluidics device according to any one of paragraphs 250-255, wherein the means for releasing the ionic solution from the lipid vesicle comprises a detergent.
257. The microfluidics device according to paragraph 256, wherein the detergent is a non-ionic detergent.
258. The microfluidics device according to any one of paragraphs 236-257, wherein at least one means for detecting the detectable label is selected from the group consisting of means for detecting a fluorescent label, means for detecting an enzymatic label, means for detecting a fluorogenic label, means for detecting a dye, means for detecting a colorimetric label, means for detecting a magnetic label, means for detecting a luminescent label, means for detecting a chemiluminescent label and means for detecting a radioactive label.
259. The microfluidics device according to any one of paragraphs 236-258, wherein the each of the plurality of detection DBDs is released from a separate channel in the microfluidics device.

### BRIEF DESCRIPTION OF DRAWINGS

FIG. 1A shows an antibody conjugate of the present disclosure. FIG. 1B shows an antibody conjugate of the present disclosure binding to a marker in a sample. FIG. 1C shows the amplification of the polynucleotide portion of the marker-bound antibody conjugate.

FIG. 2 shows a schematic side cross sectional representation of a transistor device and liposome DBD assay in accordance with embodiments of the present disclosure;

FIGS. 3A-3D show side cross sectional representations of a scheme for detection of an amplified nucleic acid molecule in solution using FETs in accordance with embodiments of the present disclosure;

FIG. 4 shows the electrical double-layer length known as the Debye limit for materials' ability to interact with a substrate interface to make a detectable change in the device voltage, in accordance with embodiments of the present disclosure.

### DETAILED DESCRIPTION OF THE DISCLOSURE

### 1. General Techniques

Unless otherwise defined herein, scientific and technical terms used in this application shall have the meanings that are commonly understood by those of ordinary skill in the art. Generally, nomenclature used in connection with, and the laboratory procedures techniques performed in pharmacology, cell and tissue culture, analytical chemistry, biochemistry, synthetic organic chemistry, medicinal and pharmaceutical chemistry, molecular biology, cell and cancer biology, neurobiology, neurochemistry, virology, immunology, microbiology, genetics and protein and nucleic acid chemistry, described herein, are those well-known and commonly used in the art. Standard techniques are used for chemical syntheses and chemical analyses. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein.

Throughout this specification and embodiments, the word “comprise,” or variations such as “comprises” or “comprising,” will be understood to imply the inclusion of a stated integer or group of integers, but not the exclusion of any other integer or group of integers. The term “including” is used to mean “including but not limited to.” “Including” and “including but not limited to” are used interchangeably.

It is understood that wherever embodiments are described herein with the language “comprising,” otherwise analogous embodiments described in terms of “consisting of” and/or “consisting essentially of” are also provided.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. As used herein, the term “about” modifying the quantity of an ingredient, parameter, calculation, or measurement in the compositions of the disclosure or employed in the methods of the disclosure refers to variation in the numerical quantity that can occur, for example, through typical measuring and liquid handling procedures used for making isolated polypeptides or pharmaceutical compositions in the real world;

through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10%, more typically still within 5%, of a given value or range. Reference to “about” a value or parameter herein includes (and describes) embodiments that are directed to that value or parameter per se. For example, description referring to “about X” includes description of “X”. Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the embodiments of the disclosure.

Exemplary methods and materials are described herein, although methods and materials similar or equivalent to those described herein can also be used in the practice or testing of the present disclosure. The materials, methods, and examples are illustrative only and not intended to be limiting.

### 2. Definitions

The term “antibody,” as used herein, refers to a gamma-globulin, or a fragment thereof, that exhibits a specific binding activity for a target molecule, namely. The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. Such antibodies may be produced in a variety of ways, including hybridoma cultures, recombinant expression in bacteria or mammalian cell cultures, and recombinant expression in transgenic animals. Also, antibodies can be produced by selecting a sequence from a library of sequences expressed in display systems such as filamentous phage, bacterial, yeast or ribosome. There is abundant guidance in the literature for selecting a particular production methodology, e.g., Chadd and Chamow, Curr. Opin. Biotechnol., 12:188-194 (2001). The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species.

The term “antibody,” as used herein, also includes the term “antigen binding fragment,” which refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments. Antibody fragments of small size, such as Fab and Fv fragments, having no effector functions and limited pharmacokinetic activity, may be generated in a bacterial expression system. Single chain Fv fragments show low immunogenicity and are cleared rapidly from the blood.

The term “aptamer,” as used herein, refers to an oligonucleotide (such as, for example, DNA or RNA) or a peptide molecule that binds to a specific target molecule. Aptamers may show a high affinity and specificity for their target molecules. Aptamers may be synthesized by chemical or enzymatic procedures, or a combination thereof. Non-limiting examples of aptamer targets include proteins, peptides, carbohydrates, and small molecules. Aptamer binding is typically determined by its tertiary structure, not its primary sequence. Target recognition and binding are usually determined by three-dimensional, shape-dependent interactions as well as hydrophobic interactions, base-stacking, and intercalation. Methods for generating aptamers that specifically bind a target molecule are known in the art. For example, the skilled artisan may generate aptamers by systematic evolution of ligands by exponential enrichment (SELEX). See, e.g., Darmostuk et al. Biotechnology Advances, 2015, vol. 33(6): 1141-1161. In some embodiments, the aptamer is an oligonucleotide. Optionally, the aptamer comprises DNA residues. In some embodiments, the aptamer comprises RNA residues. Optionally, the oligonucleotide is single-stranded.

As used herein, a “capture molecule” is a molecule used to capture the antigen or marker being assayed via an affinity binding between the capture molecule and the antigen in a liquid phase or on a solid phase. The capture molecule may be an antibody, a recombinant antibody, a protein, a recombinant protein, small or big organic molecules, or peptide or nucleic acid aptamers. If the capture molecule is an antibody, then it is named as “capture antibody.” If the capture molecule is an aptamer, then it is named as “capture aptamer.” The capture molecule may be affixed to the solid phase. In some embodiments, the capture molecule is capable of being affixed to the solid phase, e.g., upon cycling of a magnetic field or an electric current.

The terms “chelator” and “chelating agent” are used interchangeably herein and refer to a molecule that binds to metal ions and form a complex. The affinity of the chelating agent for the metal ion is measured by the dissociation constant or K_{D}. As used herein, a high-affinity chelator is typically a binding with an affinity corresponding to a K_{D} of about 10⁻⁸ M or less, about 10⁻⁹ M or less, about 10⁻¹⁰ M or less, or about 10⁻¹¹ M or less. K_{D} values are measured by techniques known by the skilled in the art, such as, the pH metric method developed by Moisescu and Pusch (Moisescu, D. G. and Pusch, H. (1975) Pfluegers Arch. 355, 243) or a modified version of said pH metric method (Smith and Miller. (1985). Biochimica et Biophysica Acta; Vol. 839, Issue 3, 287-299).

The terms “derivatized chelator” and “derivatized chelating agent” are used interchangeable herein and refer to chelators that have been chemically altered to permit them to be interposed onto a field effect transistor or a component thereof, including a substrate, a carbon nanotube, a dielectric material, a gate, or between a source and a drain. Methods for derivatizing chelators for such disposition are known in the art. For example, pyrenes are known to adsorb to carbon nanotube surfaces through π-π interactions. Additionally, azide chemistry has been demonstrated to be a powerful means to covalently modify carbon nanotubes.

As used herein, the term “calcium chelator” is used to refer to molecules that are able to bind calcium in a selective way, because they have higher affinity for calcium than for any other metal ions. Binding to calcium is typically performed through carboxylic groups.

As used herein, the term “iron chelators” is used to refer to molecules that are able to bind iron in a selective way, because they have higher affinity for iron than for any other metal ions. They typically contain oxygen, nitrogen or sulfur-donor atoms that form coordinate bonds with bound iron. The donor atoms of the ligand affect the preference of the chelator for either the Fe(II) or Fe(III) oxidation states.

As used herein, the “Debye length” (also called Debye radius), named after Peter Debye, is a measure of a charge carrier's net electrostatic effect in a solution and how far its electrostatic effect persists. A Debye sphere is a volume whose radius is the Debye length. With each Debye length, charges are increasingly electrically screened. Every Debye-length λ_{D}, the electric potential will decrease in magnitude by 1/e. Specifically, in physiological solution environments, which are relevant to many important biological, medical, and diagnostic applications, the short screening length, <1 nm, reduces the field produced by charged biomolecules at FET surface and thus makes real-time label-free detection difficult. This short screening length is also called “Debye limit” or “Debye screening limitation”.

The term “detectable label,” as used in the present disclosure, refers to a molecule with a physical property or biochemical activity that is analyzable by a detector via the label's physical property or the label's catalyzed activity. Non-limiting examples of detectable labels include fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof. A fluorogenic label may be a substrate for an enzymatic or chemical reaction that emits light following the reaction. In some embodiments, the fluorogenic label is an enzyme or a chemical reactant that causes a substrate to fluoresce following an enzymatic or chemical reaction. A colorimetric label may be a substrate for an enzymatic or chemical reaction that changes color following the reaction. In some embodiments, the colorimetric label is an enzyme or a chemical reactant that causes a substrate to change color following an enzymatic or chemical reaction. The detectable label may also be a molecule that can be detected by, e.g., a Surface Acoustic Wave (SAW) device or a Field Effect Transistor (FET). The detectable label may be contained within a lipid vesicle or displayed on the surface of the lipid vesicle. In some embodiments, the detectable label may be the lipids forming the lipid vesicle.

A “DNA-binding domain” or “DBD,” as used herein, is an independently folded protein domain that contains at least one structural motif that recognizes double- or single-stranded DNA. Preferably, the DBDs binds to the corresponding binding nucleotide sequences selectively, i.e., it observably binds to that DNA sequence despite the presence of numerous alternative candidate DNA sequences. The DBD may comprise a helix-turn-helix motif, a zinc-finger motif, a basic leucine zipper motif, or a helix-loop-helix motif.

The term “Field Effect Transistor” or “FET,” as used herein, refers to a transistor that uses an electric field to control the electrical behavior of the device. FET consists of three electrodes: source, drain, and gate. The positive gate voltage attracts electrons from the bulk to the surface of the substrate. A sufficient number of electrons induced form a thin n-channel by electrically bridging the source and drain. Otherwise, when a specific molecular recognition occurs on the gate, the FET detects the change of charge density at the interface by an electrostatic interaction with the electrons in the n-channel FETs have several advantages, including small size, low cost, and large-scale integration with other sensors and signal-processing circuits on a single chip. A skilled artisan will be able to determine which materials should be coated on the surface of the gate insulator of the FET for detection of a particular molecule. The FET may be a chelator-coated FET, such as those described in U.S. Provisional Application No. 62/718,632, U.S. Provisional Application No. 62/886,759 and PCT/US2019/046568, each of which is incorporated by reference herein in its entirety.

The term “isothermal” or “substantially isothermal,” as used in the present disclosure, describes reaction conditions that do not require thermocycling. A substantially isothermal reaction may have temperature changes at the beginning and end of an amplification reaction. For example, substantially isothermal reactions include reactions that employ a “hot start” mechanism, in which the reaction mixture is heated to a temperature necessary to activate a component of the reaction mixture and then optionally cooled to a temperature at which a nucleic acid polymerase catalyzes nucleic acid synthesis. Similarly, substantially isothermal reactions may employ a temperature to deactivate the amplification reaction, a temperature suitable for storage of the amplification products, a temperature for the release of stored reagents, or combination thereof. Thermocycling equipment can be employed to provide reaction conditions comprising a “hot start,” the reaction temperature, a deactivating temperature, or a storage temperature. The temperature at which a polymerase catalyzes the formation of a nucleic acid strand can be substantially isothermal, especially if the enzyme is active or a range of temperatures at or near its ideal polymerization temperature.

A “linker”, as used herein, is a linear chain of nucleotides of any length separating two other molecules or sequences. For example, a linker may separate the first binding nucleotide sequence from the second binding nucleotide sequence.

The term “lipid vesicle” as used in the present application, refers to spherical bilayers which are comprised of one or more lipids. As used herein, the lipid vesicles of the disclosure may also be referred to as “liposomes”. The type, number and ratio of lipids may vary with the proviso that collectively they form spherical bilayers or vesicles. The lipids may be isolated from a naturally occurring source or they may be synthesized apart from any naturally occurring source. In some embodiments, the liposome or lipid vesicle is a multilamellar vesicle (MLV), with several lamellar phase lipid bilayers. Optionally, the liposome or lipid vesicle is a small unilamellar liposome vesicle (SUV) with one lipid bilayer and a diameter typically ranging between 15-30 nm. The liposome or lipid vesicle may be a large unilamellar vesicle (LUV) with one lipid bilayer and a diameter typically ranging between 100-200 nm or larger. Lipid vesicles may be disrupted by contacting them with, e.g., a detergent. Optionally, the detergent is a non-ionic detergent.

The terms “marker” and “analyte”, as used herein, are used interchangeably and refer to one or more molecules (or signals due to such molecules in an analytical method such as mass spectrometry) that are differentially present in a sample and that are indicators of the presence of an event, condition or process. In another embodiment, the marker is a biomarker. The term “biomarker” as used herein, refers to one or more molecules (or signals due to such molecules in an analytical method such as mass spectrometry) that are differentially released into a biological fluid by any means (including secretion or by leakage through the cell membrane). The term “biomarker” also refers to a distinctive biological or biologically derived indicator of a process, event or condition. Analyte biomarkers can be used in methods of diagnosis, e.g. clinical screening, and prognosis assessment and in monitoring the results of therapy, identifying patients most likely to respond to a particular therapeutic treatment, drug screening and development. Diagnostically useful biomarkers are identified using measured levels of a single biomarker obtained from a statistically significant number of disease-negative and disease-positive subjects in a population and establishing a mean and a standard deviation for the disease negative and positive states.

A “microfluidics device” or “biochip”, as used herein, refers to a device or system that has channels and/or chambers that are generally fabricated on the micron or submicron scale. For example, the typical channels or chambers have at least one cross-sectional dimension in the range of about 0.1 microns to about 500 microns. Optionally, the cross-sectional dimension is in the range of 10 to 500, of 20 to 500, of 40 to 500, of 80 to 500, of 100 to 500, of 200 to 500, of 300 to 500, or of 400 to 500. Optionally, the cross-sectional dimension is in the range of about 0.1 to about 400 microns, of 10 to 400, of 20 to 400, of 40 to 400, of 80 to 400, of 100 to 400, of 200 to 400, of 300 to 400. Optionally, the cross-sectional dimension is in the range of about 0.1 to about 300 microns, of 10 to 300, of 20 to 300, of 40 to 300, of 80 to 300, of 100 to 300, of 200 to 300 microns. The microfluidic device comprises multiple “microfluidic channel blocks,” with fluid flow between said blocks being selectively operable. In the context of the present application, a “block” may be defined as a discrete area on the device having a microfluidic channel with a long path within a confined space.

The terms “nucleic acid molecule,” “polynucleotide,” and “oligonucleotide” are used interchangeably herein and refer to linear chains of nucleotides of any length. The nucleotides may be deoxyribonucleotides, modified nucleotides or bases, and/or their analogs, or any substrate that can be incorporated into a chain by DNA polymerase. The nucleotides may be ribonucleotides, any substrate that can be incorporated into a chain by RNA polymerase, or any substrate that can be reverse transcribed into a DNA polynucleotide. The polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in the polynucleotide need to be identical.

As used herein, a “Polymerase Chain Reaction” (PCR) amplification refers to an enzymatic nucleic acid amplification process that involves multiple cycles of denaturing template nucleic acid, annealing primers, and synthesizing a nucleic acid strand complimentary to the template strand. Each cycle involves raising and lowering the reaction temperature to provide the proper thermal environment for each step of the cycle. Denaturing template nucleic acid is usually accomplished using high temperature, while annealing primers requires a lower temperature. Synthesis of the nucleic acid complementary to the template strand will typically occur at a temperature between the temperatures used for denaturing and annealing.

The term “primer,” as defined herein, is meant to encompass any polynucleotide that is capable of priming the synthesis of a nascent polynucleotide in a template-dependent process. Sequence-specific primers should be of sufficient length to provide specific annealing to the targeted nucleotide sequence. Primers may be chemically synthesized by methods well known within the art and a skilled artisan will know how to design a successful primer set.

As used herein, “protease cleavage site” refers to an amino acid sequence that is recognized and cleaved by a protease. Non-limiting examples of protease cleavage sites include thrombin, plasmin, Factor Xa, trypsin, pepsin, Lys-N, Glu-C, caspase, Asp-N or Arg-C.

The term “sample,” as used herein, can refer to a fluid wherein the markers or biomarkers are present, or a fluid derived from the specimen into which the markers or biomarkers are initially present. In some embodiments, the sample is a biological sample into which biomarkers are released, or a fluid derived from the biological sample into which biomarkers are initially released. Such derivation may occur either in vivo or in vitro. In some instances, the biological sample is a circulating fluid such as blood or lymph, or a fraction thereof, such as serum or plasma. In other cases, the biological sample remains substantially in a particular locus, for example, synovial fluid, cerebrospinal fluid or interstitial fluid. In still further cases, the biological fluid is an excreted fluid, for example, urine, breast milk, saliva, sweat, tears, mucous, nipple aspirants, semen, vaginal fluid, pre-ejaculate and the like. A biological fluid also refers to a liquid in which cells are cultured in vitro such as a growth medium, or a liquid in which a cell sample is homogenized, such as a buffer. In some cases, the sample is a food sample or an environmental sample, such as a water or a soil sample, which contains markers or molecules to be detected.

The term “scaffold,” as used in the present disclosure, refers to a solid phase onto which the capture DBD is or can be adsorbed or immobilized. The term “solid phase” means a non-fluid substance, and includes particles (including microparticles and beads) made from materials such as polymer, metal (paramagnetic, ferromagnetic particles or beads), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase may be a stationary component, such as a surface, a membrane, a tube, a strip, a cuvette or a microtiter plate, or may be a non-stationary component, such as beads and microparticles. Microparticles can also be used as a solid phase for homogeneous assay formats. A variety of microparticles that allow either non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See for example Martin, C. R., et al., Analytical Chemistry-News & Features, May 1, 1998, 322A-327A.

The term “steric hindrance,” as used in the present embodiment, is the slowing of chemical reactions or biological processes due to steric bulk. It is usually manifested in intermolecular reactions. For example, a first DBD bound to a first binding site may prevent a second DBD from binding to a second binding site because the first DBD binds the same physical space, or a portion thereof, that the second DBD would need to occupy in order to bind the second binding site.

The term “Surface Acoustic Wave device” (SAW), as used herein, refer to mass sensors which operate with mechanical acoustic waves as their transduction mechanism and wherein the acoustic wave propagates, guided or unguided, along a single surface of the substrate. Any other Acoustic Wave biosensor can be suitable for use in the present invention, such as Bulk Acoustic Wave (BAW) devices or

Acoustic Plate Mode devices (APM), wherein in BAW devices the acoustic wave propagates unguided through the volume of the substrate and in APM devices the waves are guided by reflection from multiple surfaces. The SAW and APM devices can be grouped as Surface Generated Acoustic Wave (SGAW) devices, because both develop acoustic waves generated and detected in the surface of the piezoelectric substrate by means of Interdigital Transducers (IDTs). Examples of SGAW devices are Shear Horizontal Surface Acoustic Wave (SH-SAW), Surface Transverse Wave (STW), Love Wave (LW), Flexural Plate Wave (FPW), Shear Horizontal Acoustic Plate Mode (SH-APM) and Layered Guided Acoustic Plate Mode (LG-APM).

The terms “thermocycle” and “thermocycling,” as used herein, refer to an automated process of changing temperature at fixed time intervals during each cycle of an amplification reaction. Thermocycling is often used in PCR technique because the denaturing, annealing, and synthesizing steps typically are performed at different temperatures.

The term “uniform”, as used herein, refers to the lipid vesicles having the same size or substantially the same size. The term “uniform” may also refer to the lipid vesicle membrane comprising only one type of lipid.

### 3. Antibody-Polynucleotide Conjugates and Aptamer-Polynucleotide Conjugates

In a first aspect, the disclosure of the application provides an antibody conjugate comprising an antibody or an antigen-binding portion thereof linked to a polynucleotide. This aspect of the disclosure also provides an aptamer linked to a polynucleotide. In some embodiments, the aptamer is an oligonucleotide. Optionally, the aptamer comprises DNA residues. In some embodiments, the aptamer comprises RNA residues. Optionally, the oligonucleotide is single-stranded.

In some embodiments, the polynucleotide of the disclosure comprises a first binding nucleotide sequence (also known as a capture nucleotide sequence) that is capable of binding or specifically binds to a capture DNA binding domain (DBD), but it is not capable of binding or does not specifically bind to a detection DBD. Optionally, the polynucleotide of the disclosure also comprises a second binding nucleotide sequence (also known as a detection nucleotide sequence) that is capable of binding or specifically binds to the detection DBD, but it is not capable of binding or does not specifically binding to the capture DBD. The polynucleotide may comprise a first binding nucleotide sequence that is capable of binding to a capture DNA binding domain (DBD) but is incapable of binding to a detection DBD (i.e., a capture nucleotide sequence) and a second binding nucleotide sequence that is capable of binding to the detection DBD but is incapable of binding to the capture DBD (i.e., a detection nucleotide sequence).

The affinity binding of the marker present in the sample and bound to the antibody conjugate or aptamer conjugate of the invention, as referred in the present application, may be measured by the dissociation constant or K_{D}. In some embodiments, the antibody binds to its antigen or the aptamer binds to its target with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less. K_{D} values are measured by techniques known by the skilled in the art, such as, for example ELISA, surface plasmon resonance (SPR), fluorescence anisotropy, Bio-Layer Interferometry, typically using OCTET® technology (Octet QKe system, ForteBio®) or a KinExA® (Kinetic Exclusion Assay) assay.

Various methods to conjugate polynucleotides or oligonucleotides to antibodies and aptamers have been described in the art. In some embodiments, the antibody is linked to the polynucleotide through a protein. The protein may be protein A. Optionally, the protein is protein G. In some embodiments, the protein is protein L. DNA conjugation strategies that are available for antibodies (including antigen-binding fragments) and aptamers include non-covalent strategies, such as coupling via biotin-streptavidin (Sano, T. et al. (1992). Science 258, 120-122) or covalent conjugation, using e.g. thiol-maleimide chemistry (Agasti, S. S. et al. (2012). J. Am. Chem. Soc. 134, 18499-502). Heterobifunctional cross-linkers, such as succinimidyl 4-hydrazinonicotinate acetone hydrazone (SANH) (Mocanu, M. M. et al. (2011). Proteomics 11, 2063-70) and succinimidyl 4-(N-maleimidomethyl) cyclohexane-1-carboxylate (SMCC) (Soderberg, O. et al. (2006). Nat. Methods 3, 995-1000), may also be used to introduce a bridge between the oligonucleotide and the antibody or the aptamer. Another method that may be used is Copper-free alkyne-azide cycloaddition or click reaction based on strain-promoted alkyne-azide cycloaddition (SPAAC) (Manova, R. K. et al. (2012). Langmuir 28, 8651-63 and van Hest, J. C. et al. (2011) ChemBioChem 12, 1309-12. Commercial kits are also available for the production of oligonucleotide-conjugated antibodies and antigen-binding fragments thereof. Non-limiting examples include the Solulink Antibody-Oligonucleotide All-in-One Conjugation Kit, and the Innova Thunder-Link kit.

In some embodiments, the polynucleotide comprises deoxyribonucleic acid residues. Optionally, the polynucleotide is a DNA molecule. In some embodiments, the polynucleotide comprises ribonucleic acid residues. The polynucleotide may be a RNA molecule. Optionally, the polynucleotide comprises deoxyribonucleic acid residues and ribonucleic acid residues. In some embodiments, the sequence of nucleic acid conjugated to the antibody or the aptamer is designed to optimize the rate of isothermal PCR amplification.

Suitable DNA-binding domains to be used in the present application are DNA-binding domains which can bind with high affinity to the first or second binding nucleotide sequences linked to the antibody or aptamer.

The affinity binding of the DNA-binding domains to the polynucleotide, as referred in the present application, may be measured by the dissociation constant or K_{D}. In some embodiments, a high affinity binding corresponds to a K_{D} of at least about 10⁻⁹M, of at least 10⁻¹⁰ M, of at least 10⁻¹¹M or of at least 10⁻¹²M. K_{D} values for DNA binding proteins may be measured by techniques known by the skilled in the art, such as, for example, nitrocellulose filter binding, EMSA, DNase I footprinting, surface plasmon resonance (SPR), fluorescence anisotropy, Bio-Layer Interferometry, typically using OCTET® technology (Octet QKe system, ForteBio®) or a KinExA® (Kinetic Exclusion Assay) assay.

Other factors to consider for the DBD-polynucleotide binding include the dissociation constant k_{off} and the association constant kₒₙ. The k_{off} constant relates to the lifetime of the DBD-polynucleotide interaction and is the first-order rate constant for the dissociation of the protein-ligand complex. The k_{off} is measured in time⁻¹. Mean life of the complex, which is the average life span of the protein-ligand complexes, is calculated as 1/k_{off}. In some embodiments, the 1/k_{off} rate for the interaction of the DBD and the polynucleotide is in the range of minutes or hours. Optionally, the 1/k_{off} rate range is between 3 minutes and 60 minutes. The 1/k_{off} rate may be at least 5 minutes. Optionally, the 1/k_{off} rate range may be between 5 minutes and 40 minutes. Optionally, the 1/k_{off} rate range may be between 10 minutes and 30 minutes. In some embodiments, the 1/k_{off} rate is at least 10 minutes. Optionally, the 1/k_{off} rate range may be between 10 minutes and 20 minutes. Optionally, the 1/k_{off} rate is at least 15 minutes. Optionally, the 1/k_{off} rate range may be between 15 minutes and 30 minutes. The 1/k_{off} rate is at least 30 minutes. Optionally, the 1/k_{off} rate range may be between 30 minutes and 50 minutes. Optionally, the 1/k_{off} rate range may be between 30 minutes and 40 minutes.

The choice of the specific DNA-binding domains for the present application may be influenced by a number of considerations, including the affinity rates, the on- and off-rates of binding (such as reactions with rapid on rates and slow off rates), the ease of protein production and stability of the protein and the desired application or utility. The choice of DNA-binding domains may also be influenced by the individual DNA sequence specificity of the domain and the ability of the domain to interact with other proteins or to be influenced by a particular cellular regulatory pathway. The DNA-binding domains can be isolated from a naturally-occurring protein or may be a synthetic molecule based in whole or in part on a naturally-occurring domain. It is within the skill in the art to select a DNA-binding domain based on the above factors that is appropriate for the intended use.

Suitable component DNA-binding domains for use in the present application may be selected from any of several different classes of natural DNA-binding proteins. Optionally, the DNA-binding domains are customized or made-to-order DNA-binding domains, for instance, engineered ZFP transcription factors can be built to recognize a specific DNA sequence. In some embodiments, the DNA-binding domain is comprised of multiple reiterated modules that cooperate to achieve high-affinity binding of DNA, such as the C2H2 class of zinc-finger proteins, which typically contain a tandem array of from two or three to dozens of zinc-finger modules. Each module contains an alpha-helix capable of contacting a three base-pair stretch of DNA. Typically, at least three zinc-fingers are required for high-affinity DNA binding. Therefore, one or two zinc-fingers constitute a low affinity DNA-binding domain.

In some embodiments, the DNA-binding domain comprises a helix-turn-helix motif. DNA-binding proteins with a helix-turn-helix motif include, but are not limited to, MAT α1, MAT α2, MAT a1, Antennapedia, Ultrabithorax, Engrailed, Paired, Fushi tarazu, HOX, Unc86, Oct1, Oct2, Oct4, hRFX1, Pit, TCF-1, SRY, TrpR, RuvC, LexA, Lac I repressor, Bacteriophage Lambda Cro repressor, Bacteriophage 434 C1 and Cro repressors, P22 C2 repressor, and Bacteriophage Mu Ner protein, and variants thereof. In some embodiments, the DNA-binding domain comprises a zinc finger motif. DNA-binding proteins with zinc finger motifs include, but are not limited to, Zif268, SWI5, SIP1, FOG, Msn2p, A20, Klf4, Mac1, steroid receptors, the yeast transcriptional activator GAL4, and Krüppel and Hunchback and variants thereof. The DNA-binding domain may comprise DNA-binding proteins with the helix-loop-helix motifs. DNA-binding proteins with helix-loop-helix motifs include, but are not limited to, AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, BHLHE41, CLOCK, BMAL-1-CLOCK, EPAS1, FERD3L, FIGLA, HAND1, HAND2, HES7, HES2, HES3, HES4, HES5, HES6, HES7, HEY1, HEY2, HIF, HIF1A, ICE1, ID1, ID2, ID3, ID4, KIAA2018, LYL1, MASH1, MATH2, MAX, MESP1, MESP2, MIST1, MITF, MLX, MLXIP, MLXIPL, MNT, MOP5, MSC, MSGN1, MXD1, MXD3, MXD4, MXI1, C-Myc, N-Myc, MyoD, MYC, MYCL1, MYCL2, MYCN, MYF5, MYF6, MYOD1, MYOG, NCOA1, NCOA3, Neurogenins, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, Pho4, PTF1A, SCL, Scleraxis, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, USF2, Beta2/NeuroD1, Daughterless, Achaete-scute (T3), E12 and E47 and variants thereof. In some embodiments, the DNA-binding domain comprises a basic leucine-zipper. DNA-binding domains with a basic leucine zipper include, but are not limited to, c-Fos/c-Jun, AP-1 Fos/Jun, CREB, ATF1, ATF2, ATF4, ATF5, ATF6, ATF7, BACH1, BACH2, BATF, BATF2, CEBPA, CEBPB, CEBPD, CEBPE, CEBPG, CEBPZ, CREB1, CREB3, CREB3L1, CREB3L2, CREB3L3, CREB3L4, CREB5, CREBL1, CREM, E4BP4, FOSL1, FOSL2, GCN4, JUN, JUNB, JUND, MAFA, MAFB, NFE2, NFE2L2, NFE2L3, SNFT, XBP1 OPAQUE, NFE2L2, and Bzip Maf and variants thereof. The amino acid sequences of the component DNA-binding domains may be naturally-occurring or non-naturally-occurring (or modified).

In some embodiments, the capture DBD and the detection DBD can be selected from the same DBD family (for instance, they can both be selected from the DBD family comprising a helix-turn-helix motif, the DBD family comprising a zinc-finger motif, the DBD family comprising a basic leucine zipper motif, or the DBD family comprising a helix-loop-helix motif). Optionally, the capture DBD can be selected from one DBD family and the detection DBD can be selected from a different DBD family.

In some embodiments, the first binding nucleotide sequence (i.e., the capture nucleotide sequence) is separated from the second binding nucleotide sequence (i.e., the detection nucleotide sequence) by a linker sufficient to avoid steric hindrance between the capture DBD and the detection DBD. The linker may be at least 80 nucleotides long. In some embodiments, the linker is within the range of 25-500 nucleotides. Optionally, the linker is 25-75 nucleotides long, 25-50 nucleotides long, 30-60 nucleotides long, 50-500 nucleotides long, 100-500 nucleotides long, 200-500 nucleotides long, 300-500 nucleotides long, 400-500 nucleotides long, 25-400 nucleotides long, 50-400 nucleotides long, 100-400 nucleotides long, 200-400 nucleotides long, 300-400 nucleotides long, 25-300 nucleotides long, 50-300 nucleotides long, 100-300 nucleotides long, 200-300 nucleotides long, 25-200 nucleotides long, 50-200 nucleotides long, 100-200 nucleotides long, 25-100 nucleotides long, 35-100 nucleotides long, 45-100 nucleotides long, 55-100 nucleotides long, 65-100 nucleotides long or 75-100 nucleotides long. The length and sequence of the linker can readily be determined by one skilled in the art. In some embodiments, the linker is optimal in length and sequence for PCR amplification. In another embodiment, the length of the linker is sufficient to allow interaction of the binding nucleotide sequences with both the capture DBD and the detection DBD. In another embodiment, the linker is sufficient to allow interaction with both the capture DBD and the detection DBD, wherein a magnetic bead is bound to the capture DBD.

In some embodiments, the polynucleotide linked to the antibody or the aptamer comprises a first amplification nucleotide sequence and a second amplification nucleotide sequence, wherein the first binding nucleotide sequence and the second binding nucleotide sequence are located between the first amplification nucleotide sequence and the second amplification nucleotide sequence, i.e. the first and second binding nucleotide sequences are flanked by two amplification nucleotide sequences.

The first amplification nucleotide sequence and the second amplification nucleotide sequences may be used to amplify or replicate the polynucleotide comprising the first binding nucleotide sequence and the second binding nucleotide sequence to generate multiple copies of the polynucleotide comprising the first and second binding nucleotide sequences.

The amplification of the first and second binding nucleotide sequences present in the polynucleotide-antibody conjugate or the polynucleotide-aptamer conjugate can be performed by any technique available in the art.

In some embodiments, the amplification is performed by PCR amplification. Optionally, the amplification is performed by isothermal amplification or by an amplification substantially isothermal. Other amplification techniques that can be used to amplify the first and second binding nucleotide sequences are Loop-mediated isothermal amplification (LAMP) (Notomi T et al. Nucleic Acids Res. 2000; 28:E63) 3SR or self-sustained sequence replication (Guatelli J C et al. Proc Natl Acad Sci USA. 1990; 87:7797), Nucleic Acid Sequence-Based Amplification (NASBA) (Compton J. Nature. 1991; 350:91-2), Strand Displacement Amplification (SDA) (Walker G T. PCR Methods Appl. 1993; 3:1-6) and Rolling Circle Amplification (RCA) (Lizardi P M et al. Nat Genet. 1998; 19:225-32) and Ligase Chain Reaction (LCR) (Wiedmann M et al. PCR Methods Appl. 1994; 3:S51-64). A skilled person in the art is able to choose and synthetize the optimum nucleic acid sequence for conjugation with the antibody or the aptamer. In some embodiments, the sequence of the nucleic acid conjugated to the antibody or the aptamer is selected to optimize the rate of isothermal amplification.

In some embodiments, the polynucleotide linked to the antibody is released from the antibody upon antigen binding. Optionally, the amplification of the polynucleotide comprising the first and second binding nucleotide sequences is performed after the polynucleotide is released from the antibody. In some embodiments, the amplification of the polynucleotide comprising the first and second binding nucleotide sequences is performed with the polynucleotide linked to the antibody.

In some embodiments, the polynucleotide linked to the aptamer is released from the aptamer upon binding to its target. Optionally, the amplification of the polynucleotide comprising the first and second binding nucleotide sequences is performed after the polynucleotide is released from the aptamer. In some embodiments, the amplification of the polynucleotide comprising the first and second binding nucleotide sequences is performed with the polynucleotide linked to the aptamer.

Various methods to release a polynucleotide from an antibody or an aptamer have been described in the art. Examples of suitable methods include the cleavage of a disulfide bond between the polynucleotide and the antibody or the aptamer. The cleavage of the disulfide bond may occur via reduction. A variety of reductants can be used. Examples of reductants to be used for the cleavage of the disulfide bonds include thiols, such as β-mercaptoethanol (β-ME) or dithiothreitol (DTT). Other reductants to be used are tris(2-carboxyethyl)phosphine (TCEP) or sodium borohydride. In some embodiments, the polynucleotide is linked to the antibody or the aptamer by a peptide linker. Optionally, the polynucleotide is released by cleaving the peptide linker. The peptide linker may be cleaved by a protease. In some embodiments, the peptide linker comprises a protease cleavage site. In some embodiments, the release of the polynucleotide from the antibody or the aptamer may occur by site-specific nucleases, such as glycosidases.

In some embodiments, the antibody conjugate or aptamer conjugate of the present disclosure is used in a diagnostic marker analysis system for the detection of markers or microorganisms, providing a sensitive, specific, and robust system with small sample consumption. Said antibody conjugate or aptamer conjugate comprises (i) a polynucleotide; and (ii) an antibody linked to the polynucleotide, wherein the polynucleotide includes: a first binding nucleotide sequence that is capable of binding to a capture DNA binding domain (DBD) but incapable of binding to a detection DBD; and a second binding nucleotide sequence that is capable of binding to the detection DBD but incapable of binding to the capture DBD.

In some embodiments, the method for the detection of markers or microorganisms of the present disclosure provides a sensitive, specific, and robust sensing detection system with small sample consumption. Said method comprises:

contacting the sample with an antibody conjugate or an aptamer conjugate, wherein the antibody portion or the aptamer portion of the conjugate binds to the marker;
amplifying the polynucleotide portion of the antibody conjugate or aptamer conjugate;
binding the first nucleotide binding sequence of the amplified polynucleotide to a capture DNA binding domain (DBD), wherein the capture DBD is affixed to a scaffold;
binding the second nucleotide binding sequence of the amplified polynucleotide to a detection DBD, wherein the detection DBD is affixed to a detectable label; and
detecting the detectable label.

### 4. Methods for Detecting Markers, Microorganisms, and Viruses in a Sample

In a second aspect, the disclosure provides a method for detecting a marker in a sample. In some embodiments, the method comprises:
(a) contacting the sample with the antibody conjugate according to the first aspect of the disclosure, wherein the antibody portion of the conjugate binds to the marker;
(b) amplifying the polynucleotide portion of the antibody conjugate;
(c) binding the first nucleotide binding sequence of the amplified polynucleotide to a capture DNA binding domain (DBD), wherein the capture DBD is affixed to a scaffold;
(d) binding the second nucleotide binding sequence of the amplified polynucleotide to a detection DBD, wherein the detection DBD is affixed to a detectable label; and
(e) detecting the detectable label.

In some embodiments, the method comprises:
(a) contacting the sample with the aptamer conjugate according to the first aspect of the disclosure, wherein the aptamer portion of the conjugate binds to the marker;
(b) amplifying the polynucleotide portion of the aptamer conjugate;
(c) binding the first nucleotide binding sequence of the amplified polynucleotide to a capture DNA binding domain (DBD), wherein the capture DBD is affixed to a scaffold;
(d) binding the second nucleotide binding sequence of the amplified polynucleotide to a detection DBD, wherein the detection DBD is affixed to a detectable label; and
(e) detecting the detectable label.

In the case of the method for detection of a marker, the definitions and examples detailed above for the antibody conjugate and the aptamer conjugate apply herein.

In some embodiments, the method for detecting a marker in a sample comprises a step which involves the interaction of a sample, which includes one or more markers, with the antibody conjugate or the aptamer conjugate according to the first aspect of the disclosure. The antibody portion of the antibody conjugate or the aptamer portion of the aptamer conjugate specifically recognizes and binds the marker.

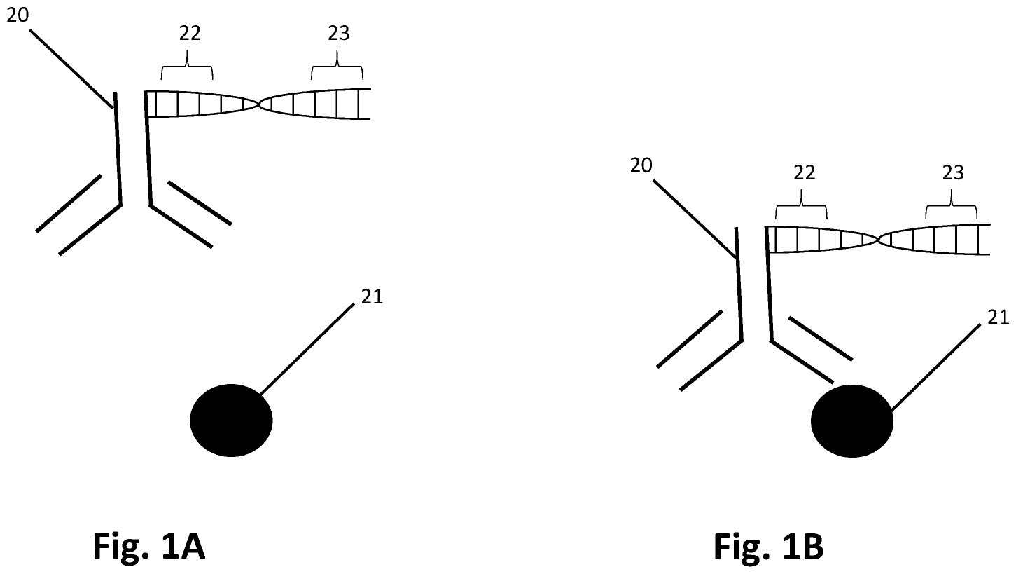

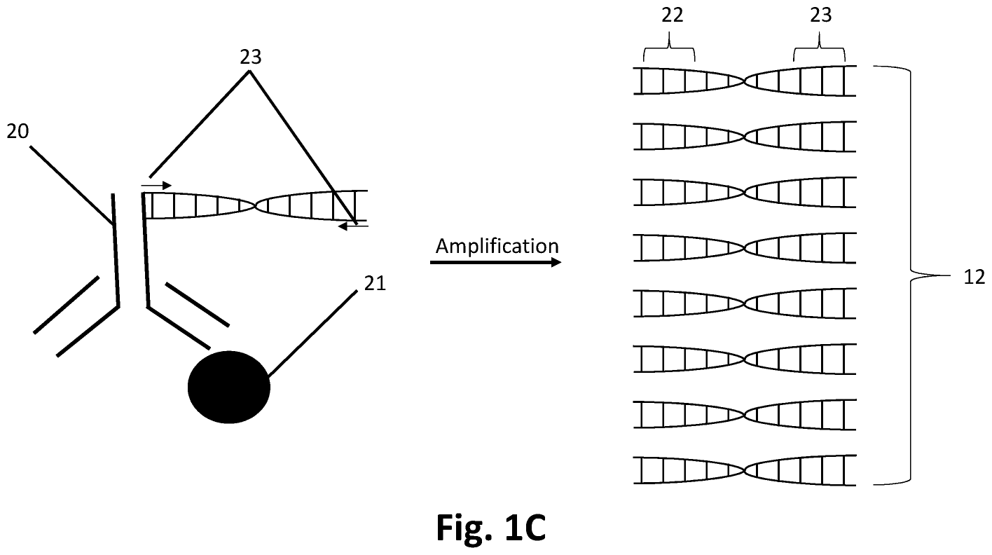

FIGS. 1 A-C show side cross sectional representations of a scheme for detection of a marker (21) in solution and binding molecule, such as an antibody conjugate (20) described herein. FIG. 1A shows an antibody conjugate of the present disclosure comprising a first binding nucleotide sequence (22) that is capable of binding to a capture DNA binding domain (DBD) but incapable of binding to a detection DBD and a second binding nucleotide sequence (23) that is capable of binding to the detection DBD but incapable of binding to the capture DBD. FIG. 1B shows the binding of the antibody conjugate (20) to the marker (21). FIG. 1C shows the amplification of the marker-bound antibody conjugate (20) using primers (23) to generate amplified polynucleotides (12) comprising a first binding nucleotide sequence (22) that is capable of binding to a capture DNA binding domain (DBD) but incapable of binding to a detection DBD and a second binding nucleotide sequence (23) that is capable of binding to the detection DBD but incapable of binding to the capture DBD.

Non-limiting examples of scaffolds to be used in the present disclosure include particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. Other particles that may be used as scaffolds include polymer particles such as polystyrene and poly(methylmethacrylate); gold particles such as gold nanoparticles and gold colloids; and ceramic particles such as silica, glass, and metal oxide particles.

In some embodiments, the method further comprises a step of washing the antibody conjugate-bound marker before the amplification step. Optionally, the method comprises a step of washing the aptamer conjugate-bound marker before the amplification step. As used in such embodiments, the washing step refers to a step to remove interfering and extraneous substances and excess antibody conjugate or aptamer not bound to the marker which may be present in the sample. Those skilled in the art will be able to determine adequate conditions to perform the washing step. Optionally, multiple steps of binding and washing may be accomplished with the use of magnetic particles or of a matrix (solid phase) through which the specimen and all subsequent mixtures/solutions are passed.

In some embodiments, the method further comprising the step of washing the amplified polynucleotide bound to the capture DBD prior to binding the detection DBD. As used in such embodiments, the washing step refers to a step to remove interfering and extraneous substances and excess amplified polynucleotide not bound to the capture DBD which may be present in the capture DBD binding step. Those skilled in the art will be able to determine adequate conditions to perform the washing step. Optionally, multiple steps of binding and washing may be accomplished with the use of magnetic particles or of a matrix (solid phase) through which the specimen and all subsequent mixtures/solutions are passed.

In some embodiments, the method further comprising the step of washing the amplified polynucleotide bound to the detection DBD prior to detecting the detectable label. As used in such embodiments, the washing step refers to a step to remove interfering and extraneous substances and excess amplified polynucleotide not bound to the detection DBD which may be present in the detection DBD binding step. Those skilled in the art will be able to determine adequate conditions to perform the washing step. Optionally, multiple steps of binding and washing may be accomplished with the use of magnetic particles or of a matrix (solid phase) through which the specimen and all subsequent mixtures/solutions are passed.

Non-limiting examples of detectable labels include fluorescent labels, fluorogenic labels, dyes, colorimetric labels, radioactive labels, luminescent labels, chemiluminescent labels, magnetic particles, metal particles, particles of 1 pg or greater, charged particles, spores and enzymatic labels.

In some embodiments, the detectable label may also be a molecule that can be detected by, e.g., a Surface Acoustic Wave (SAW) device or a Field Effect Transistor (FET). In some embodiments, the FET is a chelator-coated FET as described in US 62/718,632, U.S. Provisional Application No. 62/886,759 and PCT/US2019/046568, each of which is incorporated by reference herein in its entirety.

In some embodiments, the detectable label may be detected by a surface acoustic wave device, wherein the detectable label is selected from the group consisting of a magnetic particle, a metal particle, a particle of 1 pg or greater, a charged particle and a spore or a combination thereof.

In some embodiments, the detectable label may be detected by a field effect transistor, wherein the detectable label is selected from the group consisting of a magnetic particle, a metal particle, a charged particle and an ionic solution or a combination thereof. In some embodiments, the detectable label comprises an ionic solution. Optionally, the ionic solution comprises a metal ion. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion (e.g., As⁺³, Hg⁺², Sb⁺³, and AO. Preferably, the metal ions to be detected are divalent and trivalent ions.

In some embodiments, the detectable label is comprised within a lipid vesicle. In some embodiments, the ionic solution is comprised within a lipid vesicle. In some embodiments, the ionic solution comprises a metal ion, which is released upon disruption of the lipid vesicle.

In some embodiments, when the detectable label comprises a metal ion, the method further comprises contacting the released metal ions with a metal ion chelator or metal ion derivatized chelator located at or near the detector. In some embodiments, the metal ion chelator is attached to the surface of the detector. In some embodiments, the detector is a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, chelating agents of metallic ions include chelating agents of Ca²⁺, Fe²⁺, Fe³⁺, Mg²⁺, Mn²⁺, Cu²⁺, Cu³⁺, Zn²⁺, Ni²⁺, Co²⁺, heavy metal ions (e.g., As⁺³, Hg⁺², Sb⁺³, and AO, and the like. It is within the skill of the art to select a chelating agent or derivatized chelating agent that will bind or complex with a particular ion of interest. See, e.g., Bers D. M., MacLeod K. T. (1988) Calcium Chelators and Calcium Ionophores. In: Baker P. F. (eds) Calcium in Drug Actions. Handbook of Experimental Pharmacology, vol 83. Springer, Berlin, Heidelberg; Hatcher, H C. et al. Future Med Chem. 2009 December; 1(9): 10.4155; Sheth, S., Curr Opin Hematol 2014, 21:179; Missy P. et al. Hum Exp Toxicol., 2000, vol. 19(8): 448-456; Sigma Aldrich, BioUltra Reagents: Chelators (available at https://www.sigmaaldrich.com/life-science/metabolomics/bioultra-reagents/chelators.html); Santa Cruz Biotechnology Chelators (available at https://www.scbt.com/scbt/browse/chelators/_/N-1azot5l); Lawson M K, et al. Curr Pharmacol Rep (2016) 2:271-280; Radford and Lippard, Curr Opin Chem Biol. 2013 April; 17(2): 129-136; Chaitman, M. et al., P T. 2016 January; 41(1): 43-50, each of which is incorporated herein in its entirety.

In some embodiments, the chelating agent or derivatized chelating agent selectively binds a metal ion. Preferably, the chelating agent or derivatized chelating agent selectively binds the metal ion contained within the lipid vesicle of a detection molecule, such as a detection antibody. Optionally, the chelating agent or derivatized chelating agent binds several metal ions. The chelating agent or derivatized chelating agent may preferentially bind one metal ion, but still bind other metal ions. In some embodiments, the chelator is a custom designed chelator.

In some embodiments, the chelator is selected from the group consisting of 1,1,1-Trifluoroacetylacetone; 1,4,7-Trimethyl-1,4,7-triazacyclononane; 2,2′-Bipyrimidine; Acetylacetone; Alizarin; Amidoxime; Amidoxime group; Aminoethylethanolamine; Aminomethylphosphonic acid; Aminopolycarboxylic acid; ATMP; BAPTA; Bathocuproine; BDTH2; Benzotriazole; Bidentate; Bipyridine; 2,2′-Bipyridine; Bis(dicyclohexylphosphino)ethane; 1,2-Bis(dimethylarsino)benzene; 1,2-Bis(dimethylphosphino)ethane; 1,2-Bis(diphenylphosphino)ethane; Calixarene; Carcerand; Catechol; Cavitand; Chelating resin; Chelex 100; Citrate; Citric acid; Clathrochelate; Corrole; Cryptand; 2.2.2-Cryptand; Cyclam; Cyclen; Cyclodextrin; Deferasirox; Deferiprone; Deferoxamine; Denticity; Dexrazoxane; Diacetyl monoxime; Trans-1,2-Diaminocyclohexane; 1,2-Diaminopropane; 1,5-Diaza-3,7-diphosphacyclooctanes; 1,4-Diazacycloheptane; Dibenzoylmethane; Diethylenetriamine; Diglyme; 2,3-Dihydroxybenzoic acid; Dimercaprol; 2,3-Dimercapto-1-propanesulfonic acid; Dimercaptosuccinic acid; 1,1-Dimethylethylenediamine; 1,2-Dimethylethylenediamine; Dimethylglyoxime; DIOP; Diphenylethylenediamine; 1,5-Dithiacyclooctane; Domoic acid; DOTA; DOTA-TATE; DTPMP; EDDHA; EDDS; EDTA; EDTMP; EGTA; 1,2-Ethanedithiol; Ethylenediamine; Ethylenediaminediacetic acid; Ethylenediaminetetraacetic acid; Etidronic acid; Fluo-4; Fura-2; Gallic acid; Gluconic acid; Glutamic acid; Glyoxal-bis(mesitylimine); Glyphosate; Hexafluoroacetylacetone; Homocitric acid; Iminodiacetic acid; Indo-1; Isosaccharinic acid; Kainic acid; Ligand; Malic acid; Metal acetylacetonates; Metal dithiolene complex; Metallacrown; Nitrilotriacetic acid; Oxalic acid; Oxime; Pendetide; Penicillamine; Pentetic acid; Phanephos; Phenanthroline; O-Phenylenediamine; Phosphonate; Phthalocyanine; Phytochelatin; Picolinic acid; Polyaspartic acid; Porphine; Porphyrin; 3-Pyridylnicotinamide; 4-Pyridylnicotinamide; Pyrogallol; Salicylic acid; Sarcophagine; Sodium citrate; Sodium diethyldithiocarbamate; Sodium polyaspartate; Terpyridine; Tetramethylethylenediamine; Tetraphenylporphyrin; Thenoyltrifluoroacetone; Thioglycolic acid; TPEN; 1,4,7-Triazacyclononane; Tributyl phosphate; Tridentate; Triethylenetetramine; Triphos; Trisodium citrate; 1,4,7-Trithiacyclononane; and TTFA and derivatives thereof.

In some embodiments, the metal ion is Ca²⁺. Optionally, the chelator or the derivatized chelator for Ca²⁺ is selected from the group consisting of ethylene glycol-bis(β-aminoethyl ether)-N,N,N′,N′-tetraacetic acid (EGTA); ethylene diamine tetra acetic acid (EDTA); N-(2-Hydroxyethyl)ethylenediamine-N, N′, N′-triacetic acid Trisodium saIt (HEDTA); Nitrilotriacetic acid (NTA); BAPTA; 5,5′-dimethyl BAPTA (such as tetrapotassium salt); DMNP-EDTA; INDO 1 (such as pentapotassium salt); FURA-2 (such as pentapotassium salt); FURA 2/AM; MAPTAM; FLUO 3 (such as pentaammonium salt); Tetraacetoxymethyl Bis(2-aminoethyl) Ether N,N,N′,N′-Tetraacetic Acid; 2-{(carboxymethyl) 2-trimethylaminoethyl amino}acetic acid and salts of such agents, as well as free acids, derivatives and combinations thereof. Preferably, the chelator or the derivatized chelator for Ca²⁺ is EGTA or a derivative thereof.

Methods to determine the calcium binding affinity of EGTA are known in the art. A non-limiting example of such method is the Bers method (Bers D M. Am J Physiol. 1982; 242(5):C404-8), incorporated by reference herein in its entirety, wherein free Ca²⁺ in Ca-EGTA solutions are measured with a Ca electrode, bound Ca is calculated, and Scatchard and double-reciprocal plots are resolved for the total EGTA and the apparent Ca-EGTA association constant (Kₐₚₚ) in the solutions used. The free Ca²⁺ is then recalculated using the determined parameters, giving a more accurate knowledge of the free Ca²⁺ in these solutions and providing an accurate calibration curve for the Ca electrode. This method allows determination of free Ca²⁺, Kₐₚₚ, and total EGTA in the actual solutions used regardless of pH, temperature, or ionic strength.

In some embodiments, the metal ion is Fe²⁺ or Fe³⁺. Optionally, the chelator or derivatized chelator for Fe²⁺ or Fe³⁺ is selected from the group consisting of deferasirox; deferiprone; deferoxamine; desferrioxamine; desferrithiocin[2-(3-hydroxypyridin-2-yl)-4-methyl-4,5-dihyrothiazole-4-carboxylic acid; clioquinol; 0-trensox (Tris-N-(2-aminoethyl-[8-hydroxyquinoline-5-sulfonato-7-carboxamido] amine); tachpyr (N,N′,N″-tris(2-pyridylmethyl)-cis,cis-1,3,5-triamino-cyclohexane); dexrazoxane; triapine (3-aminopyridine-2-carboxaldehyde thiosemicarbazone); pyridoxal isonicotinoyl hydrazone; di-2-pyridylketone thiosemicarbazone series; flavan-3-ol; curcumin; apocynin; kolaviron; floranol; baicalein; baicalin; Ligusticum wallichi Francha (ligustrazine); quercetin; epigallocatechin gallate; theaflavin; phytic acid; genistein (5,7,4′-tri-hydroxyisoflavone); EDTA; NTA; HBED, o-Phenanthroline monohydrate; Pyridoxal Isonicotinoyl Hydrazone, 2,2prime-Dipyridyl, (S) 1 (p Bromoacetamidobenzyl)ethylenediaminetetraacetic Acid, (S) 1 (4 Aminoxyacetamidobenzyl)ethylenediaminetetraacetic Acid; Lipoic Acid and salts of such agents, as well as the free acids, derivatives thereof and combinations thereof.

In some embodiments, the metal ion is Mg²⁺. Optionally, the chelator or the derivatized chelator for Mg²⁺ is selected from the group consisting of EDTA, EGTA, HEDTA, NTA and salts of such agents, as well as the free acids, derivatives thereof and combinations thereof.

In some embodiments, the metal ion is Mn²⁺. Optionally, the chelator or the derivatized chelator for Mn²⁺ is selected from the group consisting of EDTA; EGTA; HEDTA; NTA; triethylenetetramine-N,N,N′,N″,N′″,N′″-hexaacetic acid (TTHA); para-aminosalicylic acid (PAS), 1,2-cyclohexylenedinitrilotetraacetic acid (CDTA), nitrilotriacetic acid (NAS), diethylenetriaminepentaacetic acid (DTPA); DPTA-OH; HBED; and salts of such agents, as well as the free acids, derivatives thereof and combinations thereof.

In some embodiments, the metal ion is Cu²⁺ or Cu³⁺. Optionally, the chelator or the derivatized chelator for Cu²⁺ or Cu³⁺ is selected from the group consisting of EDTA; NTA; D-Penicillamine (DPA); Tetraethylenetetraamine (TETA); clioquinol; glutamic acid; lipoic acid; and salts of such agents, as well as the free acids, derivatives thereof and combinations thereof.

In some embodiments, the metal ion is Zn²⁺. Optionally, the chelator or the derivatized chelator for Zn²⁺ is selected from the group consisting of ADAMTS-5 Inhibitor; N,N,N′,N-tetrakis(2-pyridinylmethyl)-1,2-ethanediamine (TPEN); EDPA; 1,2-bis(o-aminophenoxy)ethane-N,N,N′,N′-tetraacetic acid (BAPTA); CaEDTA; EDTA; EGTA; Tricine; ZX1; 4-{[2-(bis-pyridin-2-ylmethylamino)ethylamino]methyl} phenyl)methanesulfonic acid (DPESA); [4-({[2-(bis-pyridin-2-ylmethylamino)ethyl]pyridin-2-ylmethylamino}-methyl)phenyl]methanesulfonic acid (TPESA); and derivatives thereof.

In some embodiments, the metal ion is Ni²⁺. Optionally, the chelator or the derivatized chelator for Ni²⁺ is selected from the group consisting of citrate, malate, histidine, EDTA, sodium diethyldithiocarbamate (Dithiocarb), dimethyldithiocarbamate, diisopropyl, morpholine-I-dithiocarbamate, N,N′-ethylene-bis-dithiocarbamate, 2-2(oxo-1-imidazo-lidyl) ethyldithiocarbamate, dithiocarbamate, tetraethylthiuram (Antabuse), d-penicillamine, dimercaprol (BAL), N-methyl formamide, 8-Hydroxyquinoline-Cyclodextrin Conjugate, glutamic acid and salts of such agents, as well as the free acids, derivatives thereof and combinations thereof. In some embodiments the chelator or derivatized chelator for Ni²⁺ is a nickel binding protein. See, e.g., Sudan R J J, et al. (2015) Ab Initio Coordination Chemistry for Nickel Chelation Motifs. PLoS ONE 10(5): e0126787. doi:10.1371/journal.pone.0126787, incorporated by reference herein in its entirety.

In some embodiments, the metal ion is Co²⁺. Optionally, the chelator or the derivatized chelator for Co²⁺ is selected from the group consisting of L-cysteine; L-methionine; N-acetyl-cysteine; EDTA; sodium 2,3-dimercaptopropane sulfonate (DMPS); diethylenetriaminepentaacetic acid (DTPA); 2,3-dimercaptosuccinic acid (DMSA); dimercaprol; 8-Hydroxyquinoline-Cyclodextrin Conjugate; glutamic acid; deferasirox; desferrioxamine; deferiprone; and salts of such agents, as well as the free acids, derivatives thereof and combinations thereof.

In some embodiments, the metal ion is a heavy metal ion. Optionally, the heavy metal ion is selected from the group consisting of As⁺³, Hg⁺², Sb⁺³, and Au⁺. In some embodiments, the chelator or the derivatized chelator for the heavy metal ion is selected from the group consisting of Dimercaprol (2,3-dimercapto-1-propanol); Sodium 2,3 dimercaptopropanesulfonate monohydrate; 2,3-Dimercapto-1-propanesulfonic acid sodium salt; Dimercaptosuccinic acid; Penicillamine; Lipoic Acid; and salts of such agents, as well as the free acids, derivatives thereof and combinations thereof. In some embodiments, the chelator or derivatized chelator for Au⁺ comprises an SH group. Optionally, the chelator or derivatized chelator for Hg²⁺ comprises an SH group.

Methods for adding a thiol group to the chelator are known in the art. Non-limiting examples include, but are not limited, to: (a) Potassium thioacetate was added into a solution of 1,4-diioidobutane to afford the corresponding thioester. The thioester is added to a dilute solution of K₄EGTA, resulting in the formation of the mono-functionalized thioester-K₃EGTA. Thioester-K₃EGTA then reacts with KOH followed by neutralization with HCl to afford EGTA-SH; (b) Addition of 2-aminoethane-1-thiol to a solution of protected EGTA; and (c) Reaction of EGTA with 1-pyrenebutyric acid to form a thioester.

Methods for derivatizing chelators to permit disposition onto surfaces of microfluidics devices are known in the art. For example, pyrenes are known to adsorb to carbon nanotube (CNT) surfaces through π-π interactions. By reacting a chelator, such as EGTA, with 1-pyrenebutyric acid, to form the corresponding thioester, the chelator can be adsorbed to the carbon nanotube surface. Additionally, azide chemistry has been demonstrated to be a powerful means to covalently modify carbon nanotubes. Specifically, diazonium salts react with the surface of carbon nanotube surfaces to generate C—C bonds. Through the derivatization of the chelating agent with a diazonium salt, the chelator can be attached to the surface of the device. In some embodiments, the diazonium salt is 4-(2,5-dioxo-2,5-dihydro-1H-pyrrol-1-yl)benzenediazonium.

A third aspect of the present application relates to a method of detecting one of a plurality of markers in a sample. In some embodiments, the method comprises:
(a) contacting the sample with a first antibody conjugate and a second antibody conjugate, wherein the first antibody conjugate and second antibody conjugate are antibody conjugates according to the first aspect of the disclosure, wherein the antibody portion of the first antibody conjugate binds a different marker than the antibody portion of the second antibody conjugate, wherein the first binding nucleotide sequence of the polynucleotide portion of the first antibody conjugate binds to a first capture DNA binding domain (DBD) and the first binding nucleotide sequence of the polynucleotide portion of the second antibody conjugate binds to a second capture DBD, and wherein the second binding nucleotide sequence of the polynucleotide portion of the first antibody conjugate binds to a first detection DBD but not a second detection DBD, and the second binding nucleotide sequence of the polynucleotide portion of the second antibody conjugate binds to the second detection DBD but not the first detection DBD;
(b) amplifying the polynucleotide portion of the marker-bound antibody conjugate;
(c) binding the first nucleotide binding sequence of the amplified polynucleotide to a capture DBD, wherein the capture DBD binds to the first binding nucleotide sequence of the first and second antibody conjugates and is affixed to a scaffold;
(d) binding the second nucleotide binding sequence of the amplified polynucleotide to a first detection DBD, wherein the first detection DBD binds to the second binding nucleotide sequence of the first antibody conjugate and is affixed to a first detectable label;
(e) performing a first detection step to detect the first detectable label;
(f) binding the amplified polynucleotide to a second detection DBD, wherein the second detection DBD binds to the second binding nucleotide sequence of the second antibody conjugate and is affixed to a second detectable label; and
(g) performing a second detection step to detect the second detectable label.

In some embodiments, the method comprises:
(a) contacting the sample with a first aptamer conjugate and a second aptamer conjugate, wherein the first aptamer conjugate and second aptamer conjugate are aptamer conjugates according to the first aspect of the disclosure, wherein the aptamer portion of the first aptamer conjugate binds a different marker than the aptamer portion of the second aptamer conjugate, wherein the first binding nucleotide sequence of the polynucleotide portion of the first aptamer conjugate binds to a first capture DNA binding domain (DBD) and the first binding nucleotide sequence of the polynucleotide portion of the second aptamer conjugate binds to a second capture DBD, and wherein the second binding nucleotide sequence of the polynucleotide portion of the first aptamer conjugate binds to a first detection DBD but not a second detection DBD, and the second binding nucleotide sequence of the polynucleotide portion of the second aptamer conjugate binds to the second detection DBD but not the first detection DBD;
(b) amplifying the polynucleotide portion of the marker-bound aptamer conjugate;
(c) binding the first nucleotide binding sequence of the amplified polynucleotide to a capture DBD, wherein the capture DBD binds to the first binding nucleotide sequence of the first and second aptamer conjugates and is affixed to a scaffold;
(d) binding the second nucleotide binding sequence of the amplified polynucleotide to a first detection DBD, wherein the first detection DBD binds to the second binding nucleotide sequence of the first aptamer conjugate and is affixed to a first detectable label;
(e) performing a first detection step to detect the first detectable label;
(f) binding the amplified polynucleotide to a second detection DBD, wherein the second detection DBD binds to the second binding nucleotide sequence of the second aptamer conjugate and is affixed to a second detectable label; and
(g) performing a second detection step to detect the second detectable label.

The skilled artisan would recognize that in any of the methods of detecting one of a plurality of markers in a sample, a mixture of antibody conjugates and aptamer conjugates could be used. In such embodiments, the method comprises:
(a) contacting the sample with an aptamer conjugate and an antibody conjugate, wherein the aptamer conjugate and the antibody conjugate are an aptamer conjugate and an antibody conjugate according to the first aspect of the disclosure, wherein the aptamer portion of the aptamer conjugate binds a different marker than the antibody portion of the antibody conjugate, wherein the first binding nucleotide sequence of the polynucleotide portion of the aptamer conjugate binds to a first capture DNA binding domain (DBD) and the first binding nucleotide sequence of the polynucleotide portion of the antibody conjugate binds to a second capture DBD, and wherein the second binding nucleotide sequence of the polynucleotide portion of the aptamer conjugate binds to a first detection DBD but not a second detection DBD, and the second binding nucleotide sequence of the polynucleotide portion of the antibody conjugate binds to the second detection DBD but not the first detection DBD;
(b) amplifying the polynucleotide portion of the marker-bound aptamer conjugate or the marker-bound antibody conjugate;
(c) binding the first nucleotide binding sequence of the amplified polynucleotide to a capture DBD, wherein the capture DBD binds to the first binding nucleotide sequence of the aptamer and antibody conjugates and is affixed to a scaffold;
(d) binding the second nucleotide binding sequence of the amplified polynucleotide to a first detection DBD, wherein the first detection DBD binds to the second binding nucleotide sequence of the aptamer conjugate and is affixed to a first detectable label;
(e) performing a first detection step to detect the first detectable label;
(f) binding the amplified polynucleotide to a second detection DBD, wherein the second detection DBD binds to the second binding nucleotide sequence of the antibody conjugate and is affixed to a second detectable label; and
(g) performing a second detection step to detect the second detectable label.

In the case of the detection of one of a plurality of markers, the definitions and examples detailed above for the antibody conjugate and the aptamer conjugate apply herein.

In some embodiments, the first binding nucleotide sequences of the polynucleotide portions of the first and second antibody conjugates recognizes and bind to the same capture DBD. Optionally, the first binding nucleotide sequences of the polynucleotide portions of the first and second antibody conjugates recognizes and bind to different capture DBDs. In some embodiments, the first binding nucleotide sequences of the polynucleotide portions of the first and second aptamer conjugates recognizes and bind to the same capture DBD. Optionally, the first binding nucleotide sequences of the polynucleotide portions of the first and second aptamer conjugates recognizes and bind to different capture DBDs. In some embodiments, the first binding nucleotide sequences of the polynucleotide portions of the aptamer and antibody conjugates recognizes and bind to the same capture DBD. Optionally, the first binding nucleotide sequences of the polynucleotide portions of the aptamer and antibody conjugates recognizes and bind to different capture DBDs.

In some embodiments, the second binding nucleotide sequence of the polynucleotide portions of the first and second antibody conjugates recognizes and binds to different detection DBDs. In other embodiments, the second binding nucleotide sequence of the polynucleotide portions of the first and second antibody conjugates recognizes and binds to the same detection DBDs. In some embodiments, the second binding nucleotide sequence of the polynucleotide portions of the first and second aptamer conjugates recognizes and binds to different detection DBDs. In other embodiments, the second binding nucleotide sequence of the polynucleotide portions of the first and second aptamer conjugates recognizes and binds to the same detection DBDs. In some embodiments, the second binding nucleotide sequence of the polynucleotide portions of the aptamer and antibody conjugates recognizes and binds to different detection DBDs. In other embodiments, the second binding nucleotide sequence of the polynucleotide portions of the aptamer and antibody conjugates recognizes and binds to the same detection DBDs.

In some embodiments, when the first and second detection DBDs are the same DBD, the second binding sequence in the amplified polynucleotide is sequentially contacted with each of the plurality of detection DBDs and a detection step is performed between each sequential contacting step, and the marker is detected by the sequential contacting step in which the detectable label is detected. In some embodiments, the second binding sequence in the amplified polynucleotide is simultaneously contacted with each of the plurality of detection DBDs, wherein each detection DBD is in a different location (such as a channel of a microfluidics device) and a detection step is performed at each location, and the marker is detected by the location in which the detectable label is detected.

Definitions and examples of capture and detection DBDs can be found in the first aspect of the present disclosure.

In some embodiments, the method of detecting one of a plurality of markers is used for the detection of more than two markers and in this case, more than two antibody conjugates are used. A skilled person in the art will know how to adapt the present method accordingly to more than two antibody conjugates. In some embodiments, the method of detecting one of a plurality of markers is used for the detection of more than two markers and in this case, more than two aptamer conjugates are used. A skilled person in the art will know how to adapt the present method accordingly to more than two aptamer conjugates. In some methods comprising a mixture of aptamer conjugates and antibody conjugates, a plurality of aptamer conjugates may be used. A skilled person in the art will know how to adapt the present method accordingly to a plurality of aptamer conjugates. In some methods comprising a mixture of aptamer conjugates and antibody conjugates, a plurality of antibody conjugates may be used. A skilled person in the art will know how to adapt the present method accordingly to a plurality of antibody conjugates.

In certain embodiments, the polynucleotide portion of each antibody conjugate is released upon binding of each of the markers to the antibody conjugate/s. In another embodiment, each of the plurality of markers is bound to a capture molecule, wherein each capture molecule is affixed to a scaffold or capable of being affixed to a scaffold. In certain embodiments, the polynucleotide portion of each aptamer conjugate is released upon binding of each of the markers to the aptamer conjugate/s. In some embodiments, each of the plurality of markers is bound to a capture antibody, wherein each capture antibody is affixed to a scaffold or capable of being affixed to a scaffold. Optionally, each of the plurality of markers is bound to a capture aptamer, wherein each capture aptamer is affixed to a scaffold or capable of being affixed to a scaffold. In some embodiments, the plurality of markers are bound to a mixture of capture antibodies and capture aptamers, wherein each capture aptamer and each capture antibody is affixed to a scaffold or capable of being affixed to a scaffold.

In one embodiment, the method of detecting one of a plurality of markers further includes one or more washing steps, such as the following steps: a step of washing the antibody conjugate-bound markers before the amplification step and/or a step of washing the aptamer conjugate-bound markers before the amplification step and/or a step of washing the amplified polynucleotide bound to the capture DBD prior to binding the detection DBD and/or a step of washing the amplified polynucleotide bound to the first detection DBD prior to first detection step and/or a step of washing the amplified polynucleotide bound to the capture DBD after the first detection step prior to binding the second detection DBD and/or a step of washing the amplified polynucleotide bound to the second detection DBD prior to second detection step. Optionally, multiple steps of binding and washing may be accomplished with the use of magnetic particles or of a matrix (solid phase) through which the specimen and all subsequent mixtures/solutions are passed.

In another embodiment, the first detectable label and the second detectable label are different and one of the markers is detected by detecting one of the detectable labels and the other one by detecting a different detectable label than the first one.

In another embodiment, the first detectable label and the second detectable label are the same, and the marker is detected by whether the detectable label is present in the first detection step or in the second detection step.

In some embodiments, the first detectable label comprises an ionic solution. In some embodiments, the second detectable label comprises an ionic solution. In some embodiments, the first detectable label comprises an ion, e.g. a metal ion. In some embodiments, the second detectable label comprises an ion, e.g. a metal ion. In some embodiments, the first and the second detectable labels comprise a metal ion. Optionally, the metal ion of the first and second detectable label are the same ion. Optionally, the metal ion of the first and second detectable label are different ions. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, selected from the group consisting of Ca²⁺, Fe²⁺, Fe³⁺, Mg²⁺, Mn²⁺, Cu²⁺, Cu³⁺, Zn²⁺, Ni²⁺, Cu³⁺, Co²⁺ and a heavy metal ion (e.g., As⁺³, Hg⁺², Sb⁺³, and AO. Preferably, the metal ions to be detected are divalent and trivalent ions.

In some embodiments, when the first detectable label comprises a metal ion, the method further comprises contacting the released metal ions with a metal ion chelator or metal ion derivatized chelator located at or near the detector of the first detection step. In some embodiments, the metal ion chelator is attached to the surface of the detector. In some embodiments, the detector of the first detection step is a chelator-coated FET, such as those described in U.S. Provisional Application No. 62/718,632, U.S. Provisional Application No. 62/886,759 and PCT/US2019/046568, each of which is incorporated by reference herein in its entirety. In some embodiments, when the second detectable label comprises a metal ion, the method further comprises contacting the released metal ions with a metal ion chelator or metal ion derivatized chelator located at or near the detector of the second detection step. In some embodiments, the metal ion chelator is attached to the surface of the detector. In some embodiments, the detector of the second detection step is a chelator-coated FET, such as those described in U.S. Provisional Application No. 62/718,632, U.S. Provisional Application No. 62/886,759 and PCT/US2019/046568, each of which is incorporated by reference herein in its entirety. In some embodiments, the released metal ions from the first and second detectable labels are the same ions. Optionally, the released metal ions from the first and second detectable labels are different ions. Optionally, the metal ion chelator or metal ion derivatized chelator for the second detectable marker is one of the metal ion chelators described supra or derivatized therefrom.

In a fourth aspect, the present disclosure provides a method for detecting a microorganism in a sample, comprising:
(a) amplifying a polynucleotide from the microorganism with a first and second primer, wherein the first primer introduces a first binding sequence capable of binding to a capture DNA binding domain (DBD) and incapable of binding to a detection DBD and the second primer introduces a second binding sequence capable of binding the detection DBD and incapable of binding the capture DBD;
(b) binding the first binding sequence of the amplified polynucleotide to the capture DBD, wherein the capture DBD is affixed to a scaffold;
(c) binding the second binding sequence of the amplified polynucleotide to the detection DBD, wherein the detection DBD is attached to a detectable label; and
(d) detecting the label.

In some embodiments, the method for detecting a microorganism in a sample further includes a step of releasing or extracting the polynucleotide from said microorganism. Those methods are known for the skilled in the art. The methods to release or obtain a polynucleotide from a microorganism will depend on the microorganism. In some embodiments, the method to release a polynucleotide from the microorganism includes a cell disruption step. In certain embodiments, the polynucleotide is isolated or purified after the release.

Examples of methods for releasing the polynucleotide from bacteria include enzymatic, chemical or thermal lysis, mechanical disruption of the cell wall by beads or sonication, or a combination of the above (See Ahmed et al. (African Journal of Microbiology Research. Vol. 8(6), 598-602, 2014)).

Examples of methods for DNA isolation and purification for viruses can be found infra.

In some embodiments, the method for detecting a microorganism in a sample further comprises a step of isolating or purifying the polynucleotide before the amplification step.

In some embodiments, the method for detecting a microorganism in a sample further comprises a step of washing the amplified polynucleotide bound to the capture DBD. Optionally, the method for detecting a microorganism in a sample further comprises a step of washing the amplified polynucleotide bound to the detection DBD prior to detecting the presence of the detectable label. Optionally, multiple steps of binding and washing may be accomplished with the use of magnetic particles or of a matrix (solid phase) through which the specimen and all subsequent mixtures/solutions are passed.

Non-limiting examples of detectable labels include fluorescent labels, fluorogenic labels, dyes, colorimetric labels, radioactive labels, luminescent labels, chemiluminescent labels, magnetic particles, metal particles, particles of 1 pg or greater, charged particles, spores and enzymatic labels.

In some embodiments, the detectable label may also be a molecule that can be detected by, e.g., a Surface Acoustic Wave (SAW) device or a Field Effect Transistor (FET). In some embodiments, the FET is a chelator-coated FET as described in US 62/718,632, U.S. 62/886,759 and PCT/US2019/046568, each of which is incorporated by reference herein in its entirety.

In some embodiments, the detectable label may be detected by a surface acoustic wave device, wherein the detectable label is selected from the group consisting of a magnetic particle, a metal particle, a particle of 1 pg or greater, a charged particle and a spore or a combination thereof.

In some embodiments, the detectable label may be detected by a field effect transistor, wherein the detectable label is selected from the group consisting of a magnetic particle, a metal particle, a charged particle and an ionic solution or a combination thereof. In some embodiments, the detectable label comprises an ionic solution. Optionally, the ionic solution comprises a metal ion. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, the metal ion is selected from the group consisting of Ca²⁺, Fe²⁺, Fe³⁺, Mg²⁺, Mn²⁺, Cu²⁺, Cu³⁺, Zn²⁺, Ni²⁺, Cu³⁺, Co²⁺ and a heavy metal ion (e.g., As⁺³, Hg⁺², Sb⁺³, and AO. Preferably, the metal ions to be detected are divalent and trivalent ions.

In some embodiments, the detectable label is comprised within a lipid vesicle. In some embodiments, the ionic solution is comprised within a lipid vesicle. In some embodiments, the ionic solution comprises a metal ion, which is released upon disruption of the lipid vesicle.

In some embodiments, when the detectable label comprises a metal ion, the method further comprises contacting the released metal ions with a metal ion chelator or metal ion derivatized chelator located at or near the detector. In some embodiments, the metal ion chelator is attached to the surface of the detector. In some embodiments, the detector is a chelator-coated FET, such as those described in U.S. Provisional Application No. 62/718,632, U.S. Provisional Application No. 62/886,759 and PCT/US2019/046568, each of which is incorporated by reference herein in its entirety.

In the method of the detection of a microorganism, the definitions and embodiments detailed above for the method of detection of a marker apply herein.

In some embodiments, the method for detecting a microorganism in a sample is performed on a microfluidics device. Examples of microfluidics device can be found infra.

In a fifth aspect, the present disclosure provides a method for detecting a virus in a sample, comprising:
(a) amplifying a polynucleotide from the virus with a first and second primer, wherein the first primer introduces a first binding sequence capable of binding to a capture DNA binding domain (DBD) and incapable of binding to a detection DBD and the second primer introduces a second binding sequence capable of binding the detection DBD and incapable of binding the capture DBD;
(b) binding the first binding sequence of the amplified polynucleotide to the capture DBD, wherein the capture DBD is affixed to a scaffold;
(c) binding the second binding sequence of the amplified polynucleotide to the detection DBD, wherein the detection DBD is attached to a detectable label; and
(d) detecting the label.

In some embodiments, the method for detecting a virus in a sample further comprises a step of releasing the polynucleotide from a virus, which include extracting the polynucleotide from said virus. Viral DNA and RNA can be isolated from any biological material such as living or conserved tissues, cells, virus particles, or other samples for analytical or preparative purposes. In some embodiments, the viral polynucleotide extraction comprises the following steps (i) effective disruption of cells or tissue; (ii) denaturation of nucleoprotein complexes; (iii) inactivation of nucleases, for example, RNase for RNA extraction and DNase for DNA extraction and (iv) separation of the desired polynucleotide from cell debris. The resultant extracted polynucleotide may be substantially free of contaminants including protein, carbohydrate, lipids, or other nucleic acid, for example, DNA free of RNA or RNA free of DNA.

Methods of extracting a polynucleotide (DNA or RNA) from a virus are known in the art. Non-limiting examples of said methods include Guanidinium Thiocyanate-Phenol-Chloroform Extraction, Alkaline Extraction method, Ethidium Bromide (EtBr)-Cesium Chloride (CsCl) Gradient Centrifugation, IsoQuick (Orca Research, Inc., Bothell, Wash.), lysis buffer and proteinase K, MagMAX Pathogen RNA/DNA Kit (ThermoFisher Scientific) and silica gel extraction.

In some embodiments, the virus comprises RNA as its genetic material and a step of reverse transcription (RT) is performed to convert said RNA into cDNA.

In some embodiments, the method for detecting a virus in a sample further comprises a step of isolating or purifying the polynucleotide before the amplification step.

In some embodiments, the method for detecting a virus in a sample further comprises a step of washing the amplified polynucleotide bound to the capture DBD. Optionally, the method for detecting a virus in a sample further comprises a step of washing the amplified polynucleotide bound to the detection DBD prior to detecting the presence of the detectable label. Optionally, multiple steps of binding and washing may be accomplished with the use of magnetic particles or of a matrix (solid phase) through which the specimen and all subsequent mixtures/solutions are passed.

In some embodiments, the method for detecting a virus in a sample is performed on a microfluidics device. Examples of microfluidics device can be found infra.

In the method of the detection of a virus, the definitions and embodiments detailed above for the method of detection of a marker apply herein.

In a sixth aspect, the present disclosure provides a method for detecting one of a plurality of microorganisms in a sample, comprising:
(a) amplifying a polynucleotide from the plurality of microorganisms using a plurality of primer sets, each comprising a first and second primer, wherein the each primer set specifically recognizes the nucleotide sequence of a different microorganism, wherein the first primer of each primer set introduces a first binding sequence that is capable of binding to a capture DNA binding domain (DBD) and incapable of binding to any of a plurality of detection DBDs, and the second primer of each primer set introduces a second binding sequence that is capable of binding to one of the plurality of detection DBDs, wherein the first binding sequence is the same for each primer set and the second binding sequence is unique to each primer set;
(b) binding the first binding sequence of the amplified polynucleotide to the capture DBD, wherein the capture DBD is affixed to a scaffold;
(c) binding the second binding sequence of the amplified polynucleotide to a plurality of detection DBDs, wherein the detection DBDs are each attached to a detectable label; and
(d) detecting the label.

In some embodiments, the method for detecting one of a plurality of microorganisms further comprises a first step of releasing the polynucleotide from the microorganism or microorganisms present in the sample. Methods for releasing or extracting the polynucleotide from a microorganism in a sample are known in the state of the art and examples of those methods are described supra.

In some embodiments, the method for detecting one of a plurality of microorganisms further comprises a step of isolating or purifying the polynucleotide.

In some embodiments, the method for detecting one of a plurality of microorganisms comprises contacting the sample with a first antibody conjugate and a second antibody conjugate, wherein the antibody portion of the first antibody conjugate binds a different marker than the antibody portion of the second antibody conjugate, wherein the first binding nucleotide sequence of the polynucleotide portion of the first antibody conjugate binds to a first capture DNA binding domain (DBD) and the first binding nucleotide sequence of the polynucleotide portion of the second antibody conjugate binds to a second capture DBD, and wherein the second binding nucleotide sequence of the polynucleotide portion of the first antibody conjugate binds to a first detection DBD but not to a second detection DBD, and the second binding nucleotide sequence of the polynucleotide portion of the second antibody conjugate binds to the second detection DBD but not to the first detection DBD.

In some embodiments, the method for detecting one of a plurality of microorganisms comprises contacting the sample with a first aptamer conjugate and a second aptamer conjugate, wherein the aptamer portion of the first aptamer conjugate binds a different marker than the aptamer portion of the second aptamer conjugate, wherein the first binding nucleotide sequence of the polynucleotide portion of the first aptamer conjugate binds to a first capture DNA binding domain (DBD) and the first binding nucleotide sequence of the polynucleotide portion of the second aptamer conjugate binds to a second capture DBD, and wherein the second binding nucleotide sequence of the polynucleotide portion of the first aptamer conjugate binds to a first detection DBD but not to a second detection DBD, and the second binding nucleotide sequence of the polynucleotide portion of the second aptamer conjugate binds to the second detection DBD but not to the first detection DBD.

In some embodiments, the method for detecting one of a plurality of microorganisms comprises contacting the sample with an aptamer conjugate and an antibody conjugate, wherein the aptamer portion of the aptamer conjugate binds a different marker than the antibody portion of the antibody conjugate, wherein the first binding nucleotide sequence of the polynucleotide portion of the aptamer conjugate binds to a first capture DNA binding domain (DBD) and the first binding nucleotide sequence of the polynucleotide portion of the antibody conjugate binds to a second capture DBD, and wherein the second binding nucleotide sequence of the polynucleotide portion of the aptamer conjugate binds to a first detection DBD but not to a second detection DBD, and the second binding nucleotide sequence of the polynucleotide portion of the antibody conjugate binds to the second detection DBD but not to the first detection DBD.

In some embodiments, the first and second capture DBDs bind to the same first binding nucleotide sequence. In other embodiments, the first and second capture DBDs bind to different first binding nucleotide sequences. Optionally, the first and second capture DBDs are the same DBD. Optionally, the first and second capture DBDs are different DBDs.

In some embodiments, the first and second detection DBDs bind to the same second binding nucleotide sequence. In other embodiments, the first and second detection DBDs bind to different first binding nucleotide sequences. Optionally, the first and second detection DBDs are the same DBD. Optionally, the first and second detection DBDs are different DBDs.

In some embodiments, when the first and second detection DBDs are the same DBD, the second binding sequence in the amplified polynucleotide is sequentially contacted with each of the plurality of detection DBDs and a detection step is performed between each sequential contacting step, and the marker is detected by the sequential contacting step in which the detectable label is detected. In some embodiments, the second binding sequence in the amplified polynucleotide is simultaneously contacted with each of the plurality of detection DBDs, wherein each detection DBD is in a different location (such as a channel of a microfluidics device) and a detection step is performed at each location, and the microorganism is detected by the location in which the detectable label is detected.

The amplification step performed in step (a) may introduce a capture binding site by including a tail (a fragment of polynucleotides) on the forward primers (5′ end primers) recognizing the plurality of microorganisms, wherein the tail does not anneal to the polynucleotides from the plurality of microorganisms. The amplification step performed in step (a) may also introduce a detection binding site by including a tail (a fragment of polynucleotides) on the reverse primers (3′ end primers) recognizing the plurality of microorganisms, wherein the tail does not anneal to the polynucleotides from the plurality of microorganisms.

In some embodiments, the detectable label of the method for detecting one of a plurality of microorganisms is unique to the detection DBD and each different microorganism is detected by identifying which detectable label is present.

In some embodiments, each detectable label of the method for detecting one of a plurality of microorganisms is the same. Optionally, the second binding sequence in the amplified polynucleotide is sequentially contacted with each of the plurality of detection DBDs and a detection step is performed between each sequential contacting step, and the microorganism is detected by the sequential contacting step in which the detectable label is detected. In some embodiments, the second binding sequence in the amplified polynucleotide is simultaneously contacted with each of the plurality of detection DBDs, wherein each detection DBD is in a different location (such as a channel of a microfluidics device) and a detection step is performed at each location, and the microorganism is detected by the location in which the detectable label is detected.

In some embodiments, the method for detecting one of a plurality of microorganisms in a sample further comprises a step of washing the amplified polynucleotide bound to the capture DBD prior to binding to the capture DBD. Optionally, the method for detecting one of a plurality of microorganisms in a sample further comprises a step of washing the amplified polynucleotide bound to the detection DBD prior to detecting the presence of the detectable label. The method for detecting one of a plurality of microorganisms in a sample may further comprise a step of washing the amplified polynucleotide bound to the capture DBD after each sequential detection step. Optionally, multiple steps of binding and washing may be accomplished with the use of magnetic particles or of a matrix (solid phase) through which the specimen and all subsequent mixtures/solutions are passed.

In some embodiments, the method for detecting one of a plurality of microorganisms is performed on a microfluidics device. Optionally, each of the plurality of detection DBDs is released from a separate channel in the microfluidics device. Examples of microfluidics devices are provided infra.

In the method of the detection one of a plurality of microorganisms, the definitions and embodiments detailed above for the method of detection of a microorganism apply herein.

In a seventh aspect, the present disclosure provides a method for detecting one of a plurality of viruses in a sample, comprising:
(a) amplifying a polynucleotide from the plurality of viruses using a plurality of primer sets, each comprising a first and second primer, wherein the each primer set specifically recognizes the nucleotide sequence of a different microorganism, wherein the first primer of each primer set introduces a first binding sequence that is capable of binding to a capture DNA binding domain (DBD) and incapable of binding to any of a plurality of detection DBDs, and the second primer of each primer set introduces a second binding sequence that is capable of binding to one of the plurality of detection DBDs, wherein the first binding sequence is the same for each primer set and the second binding sequence is unique to each primer set;
(b) binding the first binding sequence of the amplified polynucleotide to the capture DBD, wherein the capture DBD is affixed to a scaffold;
(c) binding the second binding sequence of the amplified polynucleotide to a plurality of detection DBDs, wherein the detection DBDs are each attached to a detectable label; and
(d) detecting the label.

In some embodiments, the method for detecting one of a plurality of viruses further comprises releasing the polynucleotide from the virus or viruses present in the sample. Methods for releasing or extracting the polynucleotide from a virus in a sample are known in the state of the art and examples of those methods are described supra.

In some embodiments, the method for detecting one of a plurality of viruses further comprises a step of isolating or purifying the polynucleotide.

In some embodiments, the virus comprises RNA as its genetic material and a step of reverse transcription (RT) is performed to convert said RNA into cDNA.

In some embodiments, the detectable label of the method for detecting one of a plurality of viruses is unique to the detection DBD and each different virus is detected by identifying which detectable label is present.

In some embodiments, each detectable label of the method for detecting one of a plurality of viruses is the same. Optionally, the second binding sequence in the amplified polynucleotide is sequentially contacted with each of the plurality of detection DBDs and a detection step is performed between each sequential contacting step, and the virus is detected by the sequential contacting step in which the detectable label is detected. In some embodiments, the second binding sequence in the amplified polynucleotide is simultaneously contacted with each of the plurality of detection DBDs, wherein each detection DBD is in a different location (such as a channel of a microfluidics device) and a detection step is performed at each location, and the virus is detected by the location in which the detectable label is detected.

In some embodiments, the method for detecting one of a plurality of viruses in a sample further comprises a step of washing the amplified polynucleotide bound to the capture DBD prior to binding to the capture DBD. Optionally, the method for detecting one of a plurality of viruses in a sample further comprises a step of washing the amplified polynucleotide bound to the detection DBD prior to detecting the presence of the detectable label. The method for detecting one of a plurality of viruses in a sample may further comprise a step of washing the amplified polynucleotide bound to the capture DBD after each sequential detection step. Optionally, multiple steps of binding and washing may be accomplished with the use of magnetic particles or of a matrix (solid phase) through which the specimen and all subsequent mixtures/solutions are passed.

In some embodiments, the method for detecting one of a plurality of viruses is performed on a microfluidics device. Optionally, each of the plurality of detection DBDs is released from a separate channel in the microfluidics device. Examples of microfluidics devices are provided infra.

In the method of the detection one of a plurality of viruses, the definitions and embodiments detailed above for the method of detection of a virus apply herein.

The following embodiments apply to any of the above methods disclosed in section 4.

In some embodiments, the sample is a biological sample. Optionally, the biological sample comprises one or more markers. The sample may be a biological sample into which one or more biomarkers are released, or a fluid derived from the biological sample into which one or more biomarkers are initially released. Such derivation may occur either in vivo or in vitro. In some instances, the biological sample is a circulating fluid such as blood or lymph, or a fraction thereof, such as serum or plasma. In other embodiments, the biological sample remains substantially in a particular locus, for example, synovial fluid, cerebrospinal fluid or interstitial fluid. Optionally, the biological sample is an excreted fluid, for example, urine, breast milk, saliva, sweat, tears, mucous, nipple aspirants, semen, vaginal fluid, pre-ejaculate and the like. A biological sample may also comprise a liquid in which cells are cultured in vitro such as a growth medium, or a liquid in which a cell sample is homogenized, such as a buffer. In some embodiments, the sample is a food sample. Optionally, the sample is an environmental sample, such as a water or a soil sample, which contains markers or molecules to be detected. The sample may contain an allergen or a microorganism. In some embodiments, the microorganism is selected from the group of a bacterium, a fungus, an archaeon, an alga, a protozoan and a virus.

In some embodiments, the marker is a biomarker, an environmental marker, an allergen, or a microorganism (e.g. a bacterium, a fungus, an archaeon, an alga, a protozoan and a virus). Examples of markers which can be detected according to the methods of the present disclosure include proteins, lipids, lipoproteins, glycoproteins, nucleic acids (including circulating nucleic acids), carbohydrates, lipopolysaccharides, small molecule metabolites, and fragments thereof. The marker or biomarker may be present in the sample at a concentration that cannot be detected without signal amplification. Typically, the presence and/or the concentration of a biomarker (or biomarkers, or pattern or patterns of biomarkers) in a sample is discriminatory between physiological and pathological states of the cells from which they are released.

The amplification step can be performed according to any technique available in the art. The amplification step may be performed by isothermal amplification. In some embodiments, the amplification step is performed by PCR amplification. Optionally, the amplification step comprises the step of binding a first amplification primer to the first amplification sequence in the polynucleotide portion of the antibody conjugate and binding a second amplification primer to a second amplification sequence in the polynucleotide portion of the antibody conjugate. In some embodiments, the amplification step comprises the step of binding a first amplification primer to the first amplification sequence in the polynucleotide portion of the aptamer conjugate and binding a second amplification primer to a second amplification sequence in the polynucleotide portion of the aptamer conjugate.

In some embodiments, a multiplex PCR or a multiplex isothermal amplification is performed. As used herein, a multiplex PCR or a multiplex isothermal amplification refer to amplification techniques which are used to amplify or replicate multiple targets in a single step. In some embodiments, different primer sets are used to amplify the polynucleotide portions of the plurality of antibody conjugates. For example, two different primer sets can be used to amplify the polynucleotide portions of the first and second antibody conjugates. Optionally, the same set of primers are used to amplify the polynucleotide portions of the plurality of antibody conjugates. For example, a single set of primers can be used to amplify the polynucleotide portions of the first and second antibody conjugates. In some embodiments, different primer sets are used to amplify the polynucleotide portions of the plurality of aptamer conjugates. For example, two different primer sets can be used to amplify the polynucleotide portions of the first and second aptamer conjugates. Optionally, the same set of primers are used to amplify the polynucleotide portions of the plurality of aptamer conjugates. For example, a single set of primers can be used to amplify the polynucleotide portions of the first and second aptamer conjugates. In some embodiments, different primer sets are used to amplify the polynucleotide portions of the mixture of aptamer and antibody conjugates. For example, two different primer sets can be used to amplify the polynucleotide portions of the aptamer and antibody conjugates. Optionally, the same set of primers are used to amplify the polynucleotide portions of the mixture of aptamer and antibody conjugates. For example, a single set of primers can be used to amplify the polynucleotide portions of the aptamer and antibody conjugates. Those skilled in the art will be able to determine adequate conditions to perform the multiplex amplification step, such as the optimum primer length, temperature and specificity.

The binding step of the first nucleotide binding sequence of the amplified polynucleotide to a capture DNA binding domain (DBD) can be performed according to the any technique available in the art. The skilled artisan can readily identify conditions suitable for the binding of the capture DBD and the first nucleotide binding sequence, including temperature, buffer and pH. In some embodiments, the capture DBD is affixed to a scaffold or is capable of being affixed to a scaffold. Optionally, the scaffold is a detector for the detectable label. In some embodiments, the scaffold is adjacent to the detector for the detectable label. In some embodiments, the detection step comprises the step of transporting the detectable label to a detector for the detectable label.

In some embodiments, any of the methods of detection of markers, microorganisms or viruses includes one or more washing steps. Optionally, the washing steps can be substituted by filtering steps. In some embodiments, the methods of the disclosure further comprise a step of washing or removing the unbound material present in the sample, once the marker is bound to the capture molecule or antibody in the scaffold. The methods of the disclosure may further comprise the step of removing the unbound marker before the amplification step. In some embodiments, the method further comprises one or more steps of washing antibody conjugate-bound marker before the amplification step. In some embodiments, the method further comprises one or more steps of washing aptamer conjugate-bound marker before the amplification step. The method may further comprise the step of removing the unbound amplified polynucleotide. In some embodiments, the method further comprises one or more steps of washing the amplified polynucleotide bound to the capture DBD prior to binding the detection DBD. The method may further comprise the step of removing the unbound detection DBD. In some embodiments, the method further comprises one or more steps of washing the amplified polynucleotide bound to the detection DBD prior to detecting the detectable label.

In some embodiments, the detectable label is selected from the group of: a fluorescent label, a fluorogenic label, a dye, a colorimetric label, a radioactive label, a luminescent label, a chemiluminescent label, a magnetic particle, a metal particle, a particle of 1 pg or greater, a charged particle, a spore and an enzymatic label or combination thereof.

In some embodiments, the charged solution of the detectable label is an ionic solution. In some embodiments, the ionic solution comprises a metal ion. Optionally, the metal ion is a divalent or trivalent ion. The metal ion may be selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

A fluorogenic label may be a substrate for an enzymatic or chemical reaction that emits light following the reaction. In some embodiments, the fluorogenic label is an enzyme or a chemical reactant that causes a substrate to fluoresce following an enzymatic or chemical reaction. A luminescent label may be a substrate for an enzymatic or chemical reaction that causes luminescence following the reaction. In some embodiments, the luminescent label is an enzyme or a chemical reactant that causes a substrate to luminesce following an enzymatic or chemical reaction. A colorimetric label may be a substrate for an enzymatic or chemical reaction that changes color following the reaction. In some embodiments, the colorimetric label is an enzyme or a chemical reactant that causes a substrate to change color following an enzymatic or chemical reaction. Non-limiting examples of fluorescent labels include, but are not limited to, MDCC (Coumarin), Cy3/Cy5, Fluorescein, Rhodamine, GFP, RFP, Alexa dyes, FITC, TRITC, DyLight fluors and Qdots. Examples of enzymatic labels include horseradish peroxidase (HRP), alkaline phosphatase (AP), glucose oxidase and β-galactosidase, Formylglycine generating enzyme (FGE), Phosphopantetheinyl transferase (PPTase), Sortase, Transglutaminase, Farnesyl transferase, Biotin ligase and Lipoic acid ligase. Examples of radioactive labels include Phosphorus-32, Phosphorus-33, Hydrogen-3, Carbon-14, Sulfur-35, Yttrium-90, Gallium-68 and Iodine-125.

In some embodiments, the detectable label is capable of being detected by a Surface Acoustic Wave (SAW) device. Optionally, the detectable label is capable of being detected by a Field Effect Transistor (FET). In some embodiments, the FET is a chelator-coated FET as described in US 62/718,632, U.S. Provisional Application No. 62/886,759 and PCT/US2019/046568, each of which is incorporated by reference herein in its entirety.

In some embodiments, the detectable label is capable of being detected by a Surface Acoustic Wave (SAW) device. Non-limiting examples of labels capable of being detected by a SAW device include a magnetic particle, a metal particle, any particle of 1 pg or greater and a spore or a combination thereof.

In some embodiments, the detectable label is capable of being detected by a Field Effect Transistor (FET). Non-limiting examples of labels capable of being detected by a FET device include a charged particle, magnetic particle, a metal particle and an ionic solution or a combination thereof. In some embodiments, the detectable label comprises an ionic solution. Optionally, the ionic solution comprises a metal ion. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion (e.g., As⁺³, Hg⁺², Sb⁺³, and Au⁺). In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺. Preferably, the metal ions to be detected are divalent and trivalent ions. In some embodiments, the FET is a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, the detectable label is comprised within a lipid vesicle. In some embodiments, the ionic solution is comprised within a lipid vesicle. In some embodiments, the ionic solution comprises a metal ion, which is released upon disruption of the lipid vesicle.

In some embodiments, when the detectable label comprises a metal ion, the method further comprises contacting the released metal ions with a metal ion chelator or metal ion derivatized chelator located at or near the detector. In some embodiments, the metal ion chelator is attached to the surface of the detector. In some embodiments, the detector is a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, chelating agents of metallic ions include chelating agents of Ca²⁺, Fe²⁺, Fe³⁺, Mg²⁺, Mn²⁺, Cu²⁺, Cu³⁺, Zn²⁺, Ni²⁺, Co²⁺, heavy metal ions (e.g., As⁺³, Hg⁺², Sb⁺³, and Au⁺), and the like. It is within the skill of the art to select a chelating agent or derivatized chelating agent that will bind or complex with a particular ion of interest. See, e.g., Bers D. M., MacLeod K. T. (1988) Calcium Chelators and Calcium Ionophores. In: Baker P. F. (eds) Calcium in Drug Actions. Handbook of Experimental Pharmacology, vol 83. Springer, Berlin, Heidelberg; Hatcher, H C. et al. Future Med Chem. 2009 December; 1(9): 10.4155; Sheth, S., Curr Opin Hematol 2014, 21:179; Missy P. et al. Hum Exp Toxicol., 2000, vol. 19(8): 448-456; Sigma Aldrich, BioUltra Reagents: Chelators (available at https://www.sigmaaldrich.com/life-science/metabolomics/bioultra-reagents/chelators.html); Santa Cruz Biotechnology Chelators (available at https://www.scbt.com/scbt/browse/chelators/_/N-1azot5l); Lawson M K, et al. Curr Pharmacol Rep (2016) 2:271-280; Radford and Lippard, Curr Opin Chem Biol. 2013 April; 17(2): 129-136; Chaitman, M. et al., P T. 2016 January; 41(1): 43-50, each of which is incorporated herein in its entirety.

Other detectable labels include labels capable of being detected by surface plasmon resonance (SPR). In some embodiments, if the detectable label is a magnetic or metal particle, the solutions can be mixed by cycling an electric/magnetic field.

Examples of methods for detection of the detectable label include fluorescence, luminescence, colorimetry, radioactivity, Surface Acoustic Wave (SAW) or Surface Generated Acoustic Wave (SGAW) and Field Effect Transistor (FET).

In some embodiments, the detector is a Surface Acoustic Wave device. Any other Acoustic Wave biosensor may be used in the present disclosure, including Bulk Acoustic Wave (BAW) devices or Acoustic Plate Mode devices (APM). In BAW devices the acoustic wave propagates unguided through the volume of the substrate, and in APM devices the waves are guided by reflection from multiple surfaces. The SAW and APM devices can be combined in Surface Generated Acoustic Wave (SGAW) devices, because both develop acoustic waves generated and detected in the surface of the piezoelectric substrate by means of Interdigital Transducers (IDTs).

Example of SGAW devices, that can be used herein, include Shear

Horizontal Surface Acoustic Wave (SH-SAW), Surface Transverse Wave (STW), Love Wave (LW), Flexural Plate Wave (FPW), Shear Horizontal Acoustic Plate Mode (SH-APM) and Layered Guided Acoustic Plate Mode (LG-APM).

The input port of a SGAW sensor, comprised of metal electrodes or Interdigital Transducers (IDTs) deposited or photodesigned on an optically polished surface of a piezoelectric crystal, launches a mechanical acoustic wave into the piezoelectric material due to the inverse piezoelectric phenomenon and the acoustic wave propagates through the substrate. SAW or SGAW techniques require one binding component to be immobilized on a transducer surface, while the other binding component in buffer solution is flowed over the transducer surface. A binding interaction is detected using an acoustic method that measures small changes in the phase and amplitude of the acoustic waves that travel through the transducer sensor surface. The output signals, corresponding to changes in the phase and amplitude of waves, give information about the pure mass loading, intrinsic properties of bound materials, and viscoelastic effects such as conformational changes in protein structures, protein-protein complexes, and the internal structure of layers. These changes can be detected with network analyzers, vector voltmeters or more simple electronics, such as oscillators. These sensors offer a method for not only detection but also quantification of binding events because of being capable of measuring real-time quantitative binding affinities (KO) and kinetic constants (kₒₙ and k_{off}) of biological complexes and also concentrations of target analytes. The dimensions and physical properties of the piezoelectric substrate determine the optimal resonant frequency for the transmission of the acoustic wave and will be determined by the skilled person in the art.

The immobilization of biomolecules on the solid substrate of the transducer surface of the SGAW device helps to ensure biosensor performance, because of its role in specificity, sensitivity, reproducibility and recycling ability. In some embodiments, covalent binding is used to attach biomolecules, such as the capture molecule, the capture aptamer, the aptamer conjugate, the capture antibody or the antibody conjugate to the transducer surface. Covalent immobilization assures a reproducible, durable and stable attachment to the substrate against physico-chemical variations in the aqueous microenvironment. Self-assembled monolayer (SAM) technology provides the best results in covalent binding and allows the generation of monomolecular layers of biological molecules on a variety of substrates. Gold surfaces allow the use of functionalized thiols, whereas SiO2 surfaces enable the use of various silanes. Both methods produce monolayers of active groups for the subsequent coupling of biomolecules onto the transducer surface. A skilled person can readily determine and develop the immobilization method appropriate for every combination of biomolecule and sensor surface.

In some embodiments, the detection DBD is linked to a lipid vesicle. The lipid vesicle may comprise a detectable label, which can be contained within the vesicle or displayed on the surface of the vesicle. Optionally, the detectable label may be the lipids forming the lipid vesicle. Optionally, the lipid vesicle comprises a charged solution. In some embodiments, the charged solution is an ionic solution. In some embodiments, the ionic solution comprises a metal ion. Optionally, the metal ion is a divalent or trivalent ion. The metal ion may be selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

The type, number and ratio of lipids in the vesicle may vary with the proviso that collectively they form bilayers or vesicles. Optionally, the bilayers or vesicles are spherical. The lipids may be isolated from a naturally occurring source or they may be synthesized apart from any naturally occurring source. In a specific embodiment, the liposome or lipid vesicle is a multilamellar vesicle (MLV), with several lamellar phase lipid bilayers. In another embodiment, the liposome or lipid vesicle is a small unilamellar liposome vesicle (SUV) with one lipid bilayer and a diameter typically ranging between 15-30 nm. In another embodiment, the liposome or lipid vesicle is a large unilamellar vesicle (LUV) with one lipid bilayer and a diameter typically ranging between 100-300 nm or larger.

In some embodiments, the lipid vesicle comprises an amphipathic or amphiphilic lipid. Amphipathic and amphiphilic lipids have a hydrophilic portion and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may comprise polar or charged groups such as carbohydrates, phosphate, carboxylic, sulfate, amino, sulihydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

In some embodiments, the lipids in the lipid vesicle are phospholipids. Phospholipids include, without limitation, phosphatidic acid, phosphatidylcholine, phosphatidylethanolamine, phosphatidylglycerol, phosphatidylinositol or phosphoinositides, phosphatidylserine, and combinations thereof.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include, without limitation, dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol. In one embodiment, the lipid is a phospholipid. In another embodiment, the phospholipid is a phosphatidylcholine. In some embodiments, the lipid vesicle comprises lecithin.

Optionally, the lipid vesicles are uniform in size. Optionally, the sizes of the lipid vesicles are not uniform in size.

In some embodiments, the lipid vesicle membrane may comprise only one type of lipid. In some embodiments, all lipids present in the lipid vesicle are phospholipids. In some embodiments, all lipids present in the lipid vesicle are phosphatidylcholine. Optionally, all lipids in the lipid vesicle are lecithin. In other embodiments, the lipid vesicle membrane comprises a combination of different lipids.

The number of lipid bilayers in each vesicle may vary, with a typical range of at least 1 to about 50, or at least 1 to about 25, or at least 1 to about 15, or at least 1 to about 10, or at least 1 to about 5 lipid bilayers in each vesicle. In some embodiments, the vesicle comprises 1 lipid bilayer. Optionally, the vesicle comprises 2 lipid bilayers. The vesicle may comprise 3 lipid bilayers. In some embodiments, the vesicle comprises 4 lipid bilayers. Optionally, the vesicle comprises 5 lipid bilayers. The vesicle may comprise 6 lipid bilayers. In some embodiments, the vesicle comprises 7 lipid bilayers. Optionally, the vesicle comprises 8 lipid bilayers. The vesicle may comprise 9 lipid bilayers. In some embodiments, the vesicle comprises 10 lipid bilayers.

The diameter of the vesicles may vary. In some embodiments, the vesicles will have a diameter ranging from about 20 to about 100 nm, from about 25 to about 50 nm, from 100 to about 500 nm, from about 200 to about 500, from about 300 to about 500, from about 400 to about 500, from about 100 to about 400 nm, from about 200 to about 400, from about 300 to about 400, from about 100 to about 300 nm, from about 150 to about 300 nm, from about 200 to about 300 nm, or from about 100 to 1000 nm.

It will be understood that, in any preparation of vesicles, there will be certain heterogeneity between the vesicles relating to vesicle diameter, number of lipid bilayers, etc.

Methods for the synthesis of the lipid vesicles are known in the art. An exemplary synthesis for MLVs is as follows: Lipids and optionally other bilayer components are combined to form a homogenous mixture. This may occur through a drying step in which the lipids are dried to form a lipid film. The lipids are then combined (e.g., rehydrated) with an aqueous solvent. The aqueous solvent may have a pH in the range of about 6 to about 8, including a pH of about 7. Buffers compatible with vesicle fusion are used, typically with low concentrations of salt, such as for example bis-tris propane (BTP) buffer or PBS. The nature of the buffer may impact the length of the incubation. Accordingly, a variety of aqueous buffers may be used provided that a sufficient incubation time is also used.

In some embodiments, an ionic solution is incorporated in the resultant liposomes by including ions in the solvent for rehydration. Vesicles may be broken down to smaller sizes by vigorous mixing to obtain very large multilamellar vesicles, by sonication to obtain the smallest possible single-walled vesicles, or by various means to obtain vesicles of intermediate size and characteristics. The liposomes may be prepared in the presence of a charged solution, such as ions and, therefore, the lipid vesicles will comprise the charged solution (e.g., ionic solution) in their core. In some embodiments, the ionic solution comprises a metal ion. Optionally, the metal ion is a divalent or trivalent ion. The metal ion may be selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

The resultant MLVs may then be incubated with a crosslinker, and preferably a membrane-permeable crosslinker. The nature of the crosslinker will vary depending on the nature of the reactive groups being linked together. For example, a dithiol-containing crosslinker such as DTT or (1,4-Di-[3′-(2′-pyridyldithio)-propionamido] butane) may be used to crosslink MLVs comprised of maleimide functionalized lipids (or other functionalized lipid bilayer components), or diazide crosslinkers could be used to crosslink alkyne headgroup lipids via “click” chemistry. These various incubations are all carried out under aqueous conditions at a pH in the range of about 6 to about 8, or about 6.5 to about 7.5, or at about 7. The crosslinking step may be performed at room temperature (e.g., 20-25° C.) or at a higher temperature, including for example up to or higher than 37° C.

The resultant crosslinked lipid vesicles are then collected (e.g., by centrifugation or other pelleting means), washed with water or other aqueous buffer and then PEGylated (if needed) on their outermost or external surface by incubation with a thiol-PEG. The PEG may be of any size, including but not limited to 0.1-10 kDa, 0.5-5 kDa, or 1-3 kDa.

The vesicles may be stored at 4° C. in a buffered solution such as, but not limited to, PBS or they may be lyophilized in the presence of suitable cryopreservants and then stored at −20° C. Suitable cryopreservants include those that include sucrose.

In some embodiments, the SUV are usually prepared by sonication using, for example, a cuphorn, bath, or probe tip sonicator. Optionally, LUV are prepared by a variety of methods. Examples of those methods include extrusion (LUVET or “Large Unilamellar Vesicles prepared by Extrusion Technique”), detergent dialysis (DOV or “Di-Octylglucoside Vesicles”), fusion of SUV (FUV or “Fused Unilamellar Vesicles”), reverse evaporation (REV or “Reverse Evaporation Vesicles”), and ethanol injection.

In some embodiments, the lipid vesicles of the present application are hybrid vesicles resulting from the combined self-assembly of both amphiphilic copolymers and lipids.

Methods for the conjugation or coupling of the lipid vesicle to an antibody or an aptamer are known in the art. For example, antibodies and aptamers can be conjugated (a) directly on the phospholipid head groups of non-PEGylated liposomes; (b) conjugated directly on the phospholipid headgroups of PEGylated liposomes; or (c) conjugated on the free terminus of PEGylated chains. An example of conjugation directly on the lipid head group is the following: the antibody or aptamer to be coupled to the vesicle is modified by reaction with a bifunctional agent which reacts with a free NH2 group on the antibody and provides a free sulihydryl group available for attachment to the vesicle. The modified antibody or aptamer, which retains its chemical activity after the modification, is then reacted with the lipid vesicle containing the free sulihydryl group under conditions such that a S—S bond is formed, thereby covalently linking the antibody or aptamer to the vesicle. Examples of bifunctional agents are selected from a group comprising N-hydroxysuccinimidyl 3-(2-pyridyldithio) propionate, PDP (3-(2-pyridyldithio)propionate), maleimide, MBP, MCC derivatives and chemical analogs thereof. Other non-limiting methods to conjugate antibodies to liposomes or lipid vesicles include the following: (1) conjugation through a modified antibody or aptamer covalently bonded to avidin, which binds the surface of biotinylated liposomes or both the antibody and liposome may have conjugated biotin molecules, where the quadrivalent avidin binds the two biotinylated components; (2) conjugation through a thiol modified antibody or aptamer; (3) conjugation through a maleimide modified antibody or aptamer; (4) conjugation through an aldehyde modified antibody or aptamer to a hydrazide modified lipid; (5) conjugation through a hydrazide modified antibody or aptamer to an aldehyde modified lipid; (6) conjugation of an EDC/NHS activated PEGylated carboxylic acid modified lipid to the N-terminus of antibody or aptamer; (7) conjugation of an EDC/NHS activated (PEGylated or non-PEGylated) succinyl modified lipid to the N-terminus of antibody or aptamer; (8) conjugation of an EDC/NHS activated glutaryl modified lipid to the N-terminus of antibody or aptamer (non-PEGylated); (9) conjugation of an EDC/NHS activated dodecanoyl modified lipid to the N-terminus of antibody or aptamer (non-PEGylated); (10) conjugation of a NHS ester lipid to the N-terminus of antibody or aptamer; (11) conjugation of a cyanur modified (PEGylated or non-PEGylated) lipid to the N-terminus of antibody or aptamer; (12) conjugation through a carboxy group on an EDC/NHS activated antibody or aptamer to an amine modified PEGylated lipid; (13) conjugation through a carboxy group on an EDC/NHS activated antibody/aptamer to a phosphatidylethanolamine (PE) lipid; and (14) conjugation through a carboxy group on an EDC/NHS activated antibody or aptamer to a caproylamine lipid.

In some embodiments, the antibody or aptamer is linked to the lipid vesicle by a linker. The linker may be a peptide linker. In some embodiments, the peptide linker has a length ranging between 5 and 50 amino acids, including from about 10 to 40 amino acids or from 15 to 35 amino acids or from 20 to 30 amino acids. Optionally, the peptide linker comprises a protease cleavage site.

In some embodiments, the peptide linker comprises a protease cleavage site. Optionally, the protease cleavage sites is selected from the group consisting of thrombin, plasmin, Factor Xa, trypsin, pepsin, Lys-N, Glu-C, caspase, Asp-N or Arg-C.

In some embodiments, the linker is released by cleavage of a disulfide bond. The cleavage of the disulfide bond may occur via reduction. A variety of reductants can be used. Non-limiting examples of reductants to be used for the cleavage of the disulfide bonds include thiols, such as β-mercaptoethanol (β-ME) or dithiothreitol (DTT). In some embodiments, the reductant is tris(2-carboxyethyl)phosphine (TCEP) or sodium borohydride.

Methods for encapsulation of ions in lipid vesicles or liposomes are known in the art. Examples of these methods are detailed in McConnell and Kornberg (Biochemistry, 1971, 10 (7), pp 1111-1120), and references therein. Commercial sources are also available (such as Avanti Polar Lipids). The confirmation of successful encapsulation can be done by electrochemical methods.

In some embodiments, the lipid vesicle is stable and has no leaks over time, releasing the vesicle content only upon disruption. Optionally, at least around 90% of the detectable label remains in the vesicle over time, such as around 91%, 92%, 93%, 94%, 95%, 96%, 97%, 98%, 99% or around 100% of the detectable label remains in the vesicle. In some embodiments, the amount of detectable label in the vesicle remains stable for at least one month, such as for at least two months, for at least three months, for at least four months, for at least five months, for at least six months, for at least seven months, for at least eight months, for at least nine months, for at least ten months, for at least eleven months, for at least twelve months, for at least eighteen months, for at least twenty months or for at least twenty four months.

In some embodiments, the lipid vesicle is soluble in a detergent solution and disruption is performed by adding a detergent. In a preferred embodiment, the detergent is a non-ionic detergent. Non-ionic detergents are known by the skilled person in the art and are typically based on polyoxyethylene or a glycoside. Non-limiting examples of non-ionic detergents include Tween, Triton, Nonidet P40 (NP-40) and the Brij series. In some embodiments, the lipid vesicle is susceptible to enzymatic disruption and the disruption is performed by adding an enzyme. Optionally, the disruption is performed by adding a non-detergent chemical.

Optionally, the detectable label is capable of being detected by surface plasmon resonance (SPR). In some embodiments, if a magnetic or metal particle is used as a detectable label, the solutions can be mixed by cycling an electric/magnetic field.

In some embodiments, the polynucleotide is released upon binding of the marker to the antibody conjugate. Optionally, the polynucleotide is released upon binding of the marker to the aptamer conjugate.

In some embodiments, any of the methods to detect one or a one of a plurality of markers, or a microorganism (e.g., a virus) or one of a plurality of microorganisms is performed on a microfluidic device. Optionally, the microfluidic device comprises multiple microfluidic channel blocks, with fluid flow between said blocks being selectively operable. Said blocks may be arranged sequentially, from a first block to subsequent downstream blocks. Alternatively, blocks may form multiple branches of microfluidic channel blocks. In a further embodiment of the present method, there may be located on the microfluidic device a valve for the control of the flow of fluid within the microfluidic device. Said control may include selectively permitting the pass or block of fluid. The valve may further allow the introduction of new materials to the microfluidic device. Further still, the valve may permit the drainage of waste material from the microfluidic device. In a further embodiment such valves may be located between adjacent microfluidic channel blocks, so as to control the flow of fluid between said blocks.

In some embodiments, the method of detecting one of a plurality of markers or one of a plurality of microorganisms (e.g., a virus) is performed on a microfluidics device. Optionally, the first detection DBD is released from a first channel in the microfluidics device. The second detection DBD may be released from a second channel in the microfluidics device. In some embodiments, the release of the first detection DBD from the first channel and the first detection step occur before the release of the second detection DBD from the second channel and before the second detection step. In the case of the detection of more than two markers, the different detection DBDs are released from subsequent channels of the microfluidics device.

In another embodiment, the first and second or subsequent detection DBDs are released from the same channel in the microfluidics device, but at different times.

In some embodiments, the method comprises the following steps in a microfluidics device:
(a) providing a plurality of antibody conjugates, each comprising a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD, and an antibody specific for a marker;
(b) conjugating the antibody conjugate with the marker;
(c) separating the marker-bound antibody conjugates from unbound antibody conjugates;
(d) immobilizing the marker-bound antibody conjugates to a scaffold in an amplification chamber of the microfluidics device using antibodies (e.g., capture antibodies) specific for the marker and attached to the scaffold to create an ELISA-like sandwich;
(e) disposing a wash buffer to remove any unbound antibody conjugates that have accumulated or unattached marker-bound antibody conjugates in the amplification chamber, such that the only effective source of polynucleotides in the amplification chamber is immobilized marker-bound antibody conjugates;
(f) disposing a polymerase and primers into the amplification chamber and amplifying the polynucleotide portion of the immobilized marker-bound antibody conjugates to generate amplified polynucleotides comprising the first and second binding nucleotide sequence;
(g) disposing the amplified polynucleotides in a testing chamber of the microfluidics device;
(h) immobilizing the amplified polynucleotides to a scaffold in the testing chamber of the microfluidics device using capture DBDs that bind the first binding nucleotide sequence of the amplified polynucleotides;
(i) contacting the amplified polynucleotides with a plurality of detection DBDs, wherein each detection DBD is capable of binding the second binding nucleotide sequence of the amplified polynucleotides and is attached to a detectable label;
(j) disposing a wash buffer to remove any unbound detection DBDs that have accumulated or unattached polynucleotide-bound detection DBDs in the testing chamber, such that the only effective source of detectable label in the amplification chamber is immobilized polynucleotide-bound detection DBDs;
(k) detecting the detectable label.

In some embodiments, the method comprises the following steps in a microfluidics device:
(a) providing a marker in an amplification chamber of the microfluidics device;
(b) immobilizing the marker to a scaffold in the amplification chamber of the microfluidics device using antibodies (e.g., capture antibodies) specific for the marker and attached to the scaffold;
(c) introducing a plurality of antibody conjugates, each comprising a comprising a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD, and an antibody specific for the marker, into the amplification chamber;
(d) conjugating the antibody conjugate with the immobilized marker to form an ELISA-like sandwich;
(e) disposing a wash buffer into the amplification chamber to remove any unbound antibody conjugates that have accumulated or unattached marker-bound antibody conjugates in the amplification chamber, such that the only effective source of polynucleotides in the amplification chamber is immobilized marker-bound antibody conjugates;
(f) disposing a polymerase and primers into the amplification chamber and amplifying the polynucleotide portion of the immobilized marker-bound antibody conjugates to generate amplified polynucleotides comprising the first and second binding nucleotide sequence;
(g) disposing the amplified polynucleotides in a testing chamber of the microfluidics device;
(h) immobilizing the amplified polynucleotides to a scaffold in the testing chamber of the microfluidics device using capture DBDs that bind the first binding nucleotide sequence of the amplified polynucleotides;
(i) contacting the amplified polynucleotides with a plurality of detection DBDs, wherein each detection DBD is capable of binding the second binding nucleotide sequence of the amplified polynucleotides and is attached to a detectable label;
(j) disposing a wash buffer to remove any unbound detection DBDs that have accumulated or unattached polynucleotide-bound detection DBDs in the testing chamber, such that the only effective source of detectable label in the amplification chamber is immobilized polynucleotide-bound detection DBDs; and
(k) detecting the detectable label.

In some embodiments, the detectable label is an ion-containing lipid vesicle and method further comprises the following steps:
(l) disrupting the lipid vesicles of the immobilized polynucleotide-bound detection DBDs to release the concentration of ions into a buffer;
(m) providing an electrode isolated and covered with pure water as a continuous reference value subtracted from the measured conductivity, impedance or resistivity of the buffer to establish a delta value;
(n) providing the delta value to a microcontroller for analysis to generate a time coefficient (τ) for use in establishing reaction kinetics (k^{+/-}); and
(o) the step of measuring conductivity, impedance or resistivity of the buffer to determine the presence and/or extent of conjugation of the amplified polynucleotides to the lipid vesicles includes the steps of applying a measuring signal to a first electrode disposed in the buffer, sensing current in a second electrode disposed in the buffer according a magnitude of ions released from the lipid vesicles into the buffer, and amplifying and/or signal conditioning the sensed current for output to a detector.

In some embodiments, the disrupting step comprises introducing a liposome-disrupting solution into the testing chamber. Optionally, the liposome-disrupting solution comprises a detergent. In some embodiments, the liposome-disrupting solution comprises a liposome-disrupting enzyme.

In some embodiments, the amplification chamber and the testing chamber are the same, and the capture antibodies and the capture DBDs are affixed to different surfaces in the chamber. Optionally, the amplification chamber is different than the testing chamber, and the amplified polynucleotides are transported from the amplification chamber to the testing chamber.

In some embodiments, the amplified polynucleotides are immobilized by the capture DBDs before they are contacted with the detection DBDs. Optionally, the amplified polynucleotides are contacted with the detection DBDs before they are immobilized by the capture DBDs. In some embodiments, the detection DBDs are disposed in the amplification chamber before the amplified polynucleotides are transported to the testing chamber. Optionally, the polynucleotide-bound detection DBDs are transported from the amplification chamber to the testing chamber. In some embodiments, the detection DBDs are disposed in the testing chamber. Optionally, the detection DBDs are disposed in the testing chamber before the amplified polynucleotides are transported to the testing chamber. In some embodiments, the detection DBDs are disposed in the testing chamber after the amplified polynucleotides are transported to the testing chamber.

In some embodiments, the method comprises performing convection enhanced delivery by recirculating a buffer including the antibody conjugates multiple times through a fluidic circuit including the testing chamber to reduce time required to saturate the capture antibodies from diffusive timescales to convective timescales. Optionally, the method comprises performing convection enhanced delivery by recirculating a buffer including the marker multiple times through a fluidic circuit including the testing chamber to reduce time required to saturate the capture antibodies from diffusive timescales to convective timescales. In some embodiments, the method comprises performing convection enhanced delivery by recirculating a buffer including the detection DBDs multiple times through a fluidic circuit including the testing chamber to reduce time required to saturate the amplified polynucleotides from diffusive timescales to convective timescales. Optionally, the method comprises performing convection enhanced delivery by recirculating a buffer including the amplified polynucleotides multiple times through a fluidic circuit including the testing chamber to reduce time required to saturate the capture DBDs from diffusive timescales to convective timescales. In some embodiments, the method comprises performing convection enhanced delivery by recirculating a buffer including the polynucleotide-bound detection DBDs multiple times through a fluidic circuit including the testing chamber to reduce time required to saturate the amplified polynucleotides from diffusive timescales to convective timescales. In some embodiments, the method comprises performing convection enhanced delivery by recirculating a buffer including the detection DBDs multiple times through a fluidic circuit including the amplification chamber to reduce time required to saturate the amplified polynucleotides from diffusive timescales to convective timescales.

In some embodiments, the method further comprises disposing a wash buffer to remove any unbound antibody conjugates that have accumulated or unattached marker-bound antibody conjugates that have accumulated in the amplification chamber, such that the only effective source of polynucleotides in the amplification chamber is the immobilized (e.g. capture-antibody-bound) marker-bound antibody conjugate. Optionally, the method further comprises disposing a wash buffer to remove any unbound detection DBDs that have accumulated or unattached polynucleotide-bound detection DBDs that have accumulated in the testing chamber, such that the only effective source of detectable label in the testing chamber is the immobilized (e.g. capture-DBD-bound) polynucleotide-bound detection DBD.

In some embodiments, the marker-bound antibody conjugates are circulated in a fluidic circuit and further comprise disposing a wash buffer to remove any unattached marker-bound antibody conjugates that have accumulated in the fluidic circuit. In some embodiments, the polynucleotide-bound detection DBDs are circulated in a fluidic circuit and further comprise disposing a wash buffer to remove any unattached polynucleotide-bound detection DBDs that have accumulated in the fluidic circuit.

In some embodiments, the marker-bound antibody conjugates are circulated in a fluidic circuit and further comprising disposing a wash buffer to remove any unbound antibody conjugates that have accumulated in the fluidic circuit, before amplifying the polynucleotide portion of the immobilized marker-bound antibody conjugate, so that false positives from non-marker bound polynucleotides are not measured. In some embodiments, the polynucleotide-bound detection DBDs are circulated in a fluidic circuit and further comprising disposing a wash buffer to remove any unbound detection DBDs that have accumulated in the fluidic circuit, before detecting the detectable label of the immobilized polynucleotide-bound detection DBDs, so that false positives from unbound detection DBDs are not measured.

Another aspect included in the present disclosure is a method of improving a limit of detection (LOD) in a microfluidics device. In some embodiments, the method of improving LOD in a microfluidics device comprises:
(a) providing a plurality of antibody conjugates, each comprising a comprising a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD, and an antibody specific for the marker;
(b) conjugating the antibody conjugates with a selected marker;
(c) immobilizing the marker-bound antibody conjugates to a capture antibody affixed to a scaffold in an amplification chamber in the microfluidics device to form an ELISA-like sandwich;
(d) separating the immobilized marker-bound antibody conjugates from unbound antibody conjugates and non-immobilized antibody conjugates;
(e) disposing a polymerase and primers into the amplification chamber and amplifying the polynucleotide portion of the antibody conjugates to produce amplified polynucleotides comprising the first and second binding nucleotide sequences;
(f) disposing the amplified polynucleotides in a testing chamber of the microfluidics device;
(g) immobilizing the amplified polynucleotides to a scaffold in the testing chamber of the microfluidics device using capture DBDs that bind the first binding nucleotide sequence of the amplified polynucleotides;
(h) contacting the amplified polynucleotides with a plurality of detection DBDs, wherein each detection DBD is capable of binding the second binding nucleotide sequence of the amplified polynucleotides and is attached to a detectable label;
(i) disposing a wash buffer to remove any unbound detection DBDs that have accumulated or unattached polynucleotide-bound detection DBDs in the testing chamber, such that the only effective source of detectable label in the amplification chamber is immobilized polynucleotide-bound detection DBDs;
(j) detecting the detectable label.

In some embodiments, the method of improving LOD in a microfluidics device comprises:
(a) disposing a marker into an amplification chamber in the microfluidics device;
(b) immobilizing the marker using antibodies (e.g., capture antibodies) specific for the marker and affixed to a scaffold in the amplification chamber;
(c) providing a plurality of antibody conjugates, each comprising a comprising a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD, and an antibody specific for the marker, into the testing chamber;
(d) conjugating the antibody conjugates with the marker to form an ELISA-like sandwich;
(e) separating the immobilized marker-bound antibody conjugates from unbound antibody conjugates and non-immobilized antibody conjugates;
(f) disposing a polymerase and primers into the amplification chamber and amplifying the polynucleotide portion of the antibody conjugates to produce amplified polynucleotides comprising the first and second binding nucleotide sequences;
(g) disposing the amplified polynucleotides in a testing chamber of the microfluidics device;
(h) immobilizing the amplified polynucleotides to a scaffold in the testing chamber of the microfluidics device using capture DBDs that bind the first binding nucleotide sequence of the amplified polynucleotides;
(i) contacting the amplified polynucleotides with a plurality of detection DBDs, wherein each detection DBD is capable of binding the second binding nucleotide sequence of the amplified polynucleotides and is attached to a detectable label;
(j) disposing a wash buffer to remove any unbound detection DBDs that have accumulated or unattached polynucleotide-bound detection DBDs in the testing chamber, such that the only effective source of detectable label in the amplification chamber is immobilized polynucleotide-bound detection DBDs;
(k) detecting the detectable label.

In some embodiments, the detectable label is an ion-containing lipid vesicle and method further comprises the following steps:
(e) disrupting the lipid vesicles of the immobilized polynucleotide-bound detection DBDs to release the concentration of ions into a buffer;
(f) providing an electrode isolated and covered with pure water as a continuous reference value subtracted from the measured conductivity, impedance or resistivity of the buffer to establish a delta value;
(g) providing the delta value to a microcontroller for analysis to generate a time coefficient (τ) for use in establishing reaction kinetics (k+/−); and
(h) the step of measuring conductivity, impedance or resistivity of the buffer to determine the presence and/or extent of conjugation of the amplified polynucleotides to the lipid vesicles includes the steps of applying a measuring signal to a first electrode disposed in the buffer, sensing current in a second electrode disposed in the buffer according a magnitude of ions released from the lipid vesicles into the buffer, and amplifying and/or signal conditioning the sensed current for output to a detector.

In some embodiments, the disrupting step comprises introducing a liposome-disrupting solution into the testing chamber. Optionally, the liposome-disrupting solution comprises a detergent. In some embodiments, the liposome-disrupting solution comprises a liposome-disrupting enzyme.

In some embodiments, the amplification chamber and the testing chamber are the same, and the capture antibodies and the capture DBDs are affixed to different surfaces in the chamber. Optionally, the amplification chamber is different than the testing chamber, and the amplified polynucleotides are transported from the amplification chamber to the testing chamber.

In some embodiments, the amplified polynucleotides are immobilized by the capture DBDs before they are contacted with the detection DBDs. Optionally, the amplified polynucleotides are contacted with the detection DBDs before they are immobilized by the capture DBDs. In some embodiments, the detection DBDs are disposed in the amplification chamber before the amplified polynucleotides are transported to the testing chamber. Optionally, the polynucleotide-bound detection DBDs are transported from the amplification chamber to the testing chamber. In some embodiments, the detection DBDs are disposed in the testing chamber. Optionally, the detection DBDs are disposed in the testing chamber before the amplified polynucleotides are transported to the testing chamber. In some embodiments, the detection DBDs are disposed in the testing chamber after the amplified polynucleotides are transported to the testing chamber.

In some embodiments, the method comprises performing convection enhanced delivery by recirculating a buffer including the antibody conjugates multiple times through a fluidic circuit including the testing chamber to reduce time required to saturate the capture antibodies from diffusive timescales to convective timescales. Optionally, the method comprises performing convection enhanced delivery by recirculating a buffer including the marker multiple times through a fluidic circuit including the testing chamber to reduce time required to saturate the capture antibodies from diffusive timescales to convective timescales. In some embodiments, the method comprises performing convection enhanced delivery by recirculating a buffer including the detection DBDs multiple times through a fluidic circuit including the testing chamber to reduce time required to saturate the amplified polynucleotides from diffusive timescales to convective timescales. Optionally, the method comprises performing convection enhanced delivery by recirculating a buffer including the amplified polynucleotides multiple times through a fluidic circuit including the testing chamber to reduce time required to saturate the capture DBDs from diffusive timescales to convective timescales. In some embodiments, the method comprises performing convection enhanced delivery by recirculating a buffer including the polynucleotide-bound detection DBDs multiple times through a fluidic circuit including the testing chamber to reduce time required to saturate the amplified polynucleotides from diffusive timescales to convective timescales. In some embodiments, the method comprises performing convection enhanced delivery by recirculating a buffer including the detection DBDs multiple times through a fluidic circuit including the amplification chamber to reduce time required to saturate the amplified polynucleotides from diffusive timescales to convective timescales.

In some embodiments, the method further comprises disposing a wash buffer to remove any unbound antibody conjugates that have accumulated or unattached marker-bound antibody conjugates that have accumulated in the amplification chamber, such that the only effective source of polynucleotides in the amplification chamber is the immobilized (e.g. capture-antibody-bound) marker-bound antibody conjugate. Optionally, the method further comprises disposing a wash buffer to remove any unbound detection DBDs that have accumulated or unattached polynucleotide-bound detection DBDs that have accumulated in the testing chamber, such that the only effective source of detectable label in the testing chamber is the immobilized (e.g. capture-DBD-bound) polynucleotide-bound detection DBD.

In some embodiments, the marker-bound antibody conjugates are circulated in a fluidic circuit and further comprise disposing a wash buffer to remove any unattached marker-bound antibody conjugates that have accumulated in the fluidic circuit. In some embodiments, the polynucleotide-bound detection DBDs are circulated in a fluidic circuit and further comprise disposing a wash buffer to remove any unattached polynucleotide-bound detection DBDs that have accumulated in the fluidic circuit.

In some embodiments, the marker-bound antibody conjugates are circulated in a fluidic circuit and further comprising disposing a wash buffer to remove any unbound antibody conjugates that have accumulated in the fluidic circuit, before amplifying the polynucleotide portion of the immobilized marker-bound antibody conjugate, so that false positives from non-marker bound polynucleotides are not measured. In some embodiments, the polynucleotide-bound detection DBDs are circulated in a fluidic circuit and further comprising disposing a wash buffer to remove any unbound detection DBDs that have accumulated in the fluidic circuit, before detecting the detectable label of the immobilized polynucleotide-bound detection DBDs, so that false positives from unbound detection DBDs are not measured.

In some embodiments, the method further comprises providing an electrode isolated and covered with pure water as a continuous reference value subtracted from the measured conductivity, impedance or resistivity of the buffer to establish a delta value.

In some embodiments, the method further comprises providing the delta value to a microcontroller for analysis to generate a time coefficient (τ) for use in establishing reaction kinetics (k+/−).

In some embodiments, the method further comprises a step of measuring conductivity, impedance or resistivity of the buffer to determine the presence and/or extent of conjugation of the marker to the liposomes comprises applying a measuring signal to a first electrode disposed in the buffer, sensing current in a second electrode disposed in the buffer according a magnitude of ions released from the marker ELISA sandwich into the buffer, and amplifying and/or signal conditioning the sensed current for output to a detector.

### 5. Microfluidics Device for Detecting Markers, Microorganisms and Viruses

In an eighth aspect, the present disclosure provides a microfluidics device comprising:
(a) means for receiving a sample;
(b) an antibody conjugate, wherein the conjugate comprises an antibody linked to a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD;
(c) means for contacting the sample with the antibody conjugate;
(d) means for amplifying the polynucleotide portion of the antibody conjugate;
(e) the capture DBD affixed to a scaffold;
(f) means for contacting the amplified polynucleotide with the capture DBD;
(g) the detection DBD attached to a detectable label, wherein the capture DBD and the detection DBD are different;
(h) means for contacting the amplified polynucleotide with the detection DBD; and
(i) means for detecting the detectable label.

As used herein, “means for contacting the sample with the antibody conjugate” refers to providing the adequate conditions and parameters, such as antibody concentration, temperature, pH, buffer, etc., so that the antibody conjugate specifically recognizes and binds the sample antigen. In some embodiments, the means for contacting the sample with the antibody conjugate comprises moving the sample into a channel of the microfluidics device containing the antibody conjugate. Optionally, the means for contacting the sample with the antibody conjugate comprises moving the antibody into a channel of the microfluidics device containing the sample. The means for contacting the sample with the antibody conjugate may comprise simultaneously moving the sample from a first channel of the microfluidics device into a second channel of the microfluidics device and the antibody conjugate from a third channel of the microfluidics device into the second channel.

In some embodiments, the microfluidic device comprises means for immobilizing the marker. Optionally, the means for immobilizing the marker is a capture molecule that binds the marker and is affixed to a solid phase or is capable of being affixed to a solid phase. Optionally, the solid phase comprises particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase may be a stationary component, such as a surface, a membrane, a tube, a strip, a cuvette or a microtiter plate, or may be a non-stationary component, such as beads and microparticles. Microparticles can also be used as a solid phase for homogeneous assay formats. A variety of microparticles that allow either non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See for example Martin, C. R., et al., Analytical Chemistry-News & Features, May 1, 1998, 322A-327A.

In some embodiments, the antibody conjugate is affixed to the solid phase. Optionally, the antibody conjugate is capable of being affixed to the solid phase. For example, the antibody conjugate may be linked to a magnetic bead or a metallic particle, which will bind the solid phase upon the application of an electric current or a magnetic field. In such embodiments, the cycling of the electric current or the magnetic field can be used to mix the solutions within the microfluidics device.

In some embodiments, the microfluidic device comprises means for washing or filtering. In some embodiments, the microfluidic device comprises means for washing the immobilized marker, once the marker is bound to the capture molecule in the scaffold, in order to remove unbound materials present in the sample. The means for washing the immobilized marker may comprise moving one or more wash buffers into the channel of the microfluidics device containing the immobilized marker and removing the one or more wash buffers from that channel Optionally, the means for washing the immobilized marker comprises moving the immobilized marker through one or more channels comprising a wash buffer. In some embodiments, the means for washing the immobilized marker comprises moving the immobilized marker into a channel comprising a wash buffer and removing the wash buffer from that channel Optionally, the means for washing the immobilized marker comprises moving the wash buffer into a channel comprising the immobilized marker and removing the immobilized marker from that channel Optionally, multiple steps of binding and washing may be accomplished with the use of magnetic particles or of a matrix (solid phase) through which the specimen and all subsequent mixtures/solutions are passed.

The skilled artisan would recognize that the antibody conjugate of the eighth aspect of the disclosure may be replaced with an aptamer conjugate. Definitions and Examples of the antibody, antibody conjugate, aptamer, aptamer conjugate, amplification techniques, the capture DBD, the detection DBD, the detectable label, scaffolds, SAW and FET techniques, can be found in the earlier aspects of the disclosure (antibody-polynucleotide conjugate aspect and methods to detect a marker or microorganism).

In a ninth aspect of the present application, the present disclosure provides a microfluidics device comprising:
(a) means for receiving a sample;
(b) a first antibody conjugate, wherein the first antibody conjugate comprises a first antibody linked to a first polynucleotide, wherein the first polynucleotide comprises a capture nucleotide sequence that is capable of binding to a capture DNA binding domain (DBD) and is incapable of binding to a first or second detection DBD and a first detection nucleotide sequence that is capable of binding to the first detection DBD and is incapable of binding to the capture DBD or the second detection DBD;
(c) a second antibody conjugate, wherein the second antibody conjugate comprises a second antibody linked to a second polynucleotide, wherein the second polynucleotide comprises a capture nucleotide sequence that is capable of binding to the capture DBD and is incapable of binding to the first or second detection DBD and a second detection nucleotide sequence that is capable of binding to the second detection DBD and is incapable of binding to the capture DBD or the first detection DBD;
(d) means for contacting the sample with the antibody conjugates;
(e) means for amplifying the polynucleotide portions of the first and second antibody conjugates;
(f) the capture DBD affixed to a scaffold;
(g) means for contacting the amplified polynucleotides with the capture DBD;
(h) the first detection DBD attached to a first detectable label, the second detection DBD attached to a second detectable label;
(i) means for contacting the amplified polynucleotides with the first and second detection DBDs; and
(j) means for detecting the first and second detectable labels.

The skilled artisan would recognize that the antibody conjugates of the ninth aspect of the disclosure may be replaced with aptamer conjugates or a mixture of antibody conjugates and aptamer conjugates.

In some embodiments, the microfluidics device as disclosed herein, comprises two or more than two of the antibody conjugates, wherein each of the one or more additional antibody conjugates comprises an antibody linked to one or more additional polynucleotides, wherein each of the one or more additional polynucleotides comprises a capture nucleotide sequence that recognizes and binds or is capable of binding to the capture DBD and does not recognize and bind or is capable of binding to the first, second, or any additional detection DBD, and an additional detection nucleotide sequence that recognizes and binds or is capable of binding to the one or more additional detection DBDs and does not recognize and bind or is capable of binding to the capture DBD, the first, second or any other additional detection DBD.

As used herein, “means for contacting the sample with the antibody conjugates” refers to providing the adequate conditions and parameters, such as antibody concentration, temperature, pH, buffer, etc., so that the first and second or subsequent antibody conjugates specifically recognize and bind the sample antigens.

In some embodiments, the means for contacting the sample with the antibody conjugates comprises moving the sample into a channel of the microfluidics device containing the first and second antibody conjugates. Optionally, the means for contacting the sample with the antibody conjugate comprises moving the sample into a first channel of the microfluidics device containing the first antibody conjugate and to a second channel of the microfluidics device containing the second antibody conjugate.

In some embodiments, the means for contacting the sample with the antibody conjugates comprises moving the first and second antibody conjugates into a channel of the microfluidics device containing the sample.

The means for contacting the sample with the antibody conjugates may comprise moving the sample from a first channel of the microfluidics device into a second channel of the microfluidics device and the first and second antibody conjugates from a third channel of the microfluidics device into the second channel Optionally, the means for contacting the sample with the antibody conjugates may comprise moving the sample from a first channel of the microfluidics device into a second channel of the microfluidics device, the first antibody conjugate from a third channel of the microfluidics device into the second channel and the second antibody conjugate from a fourth channel of the microfluidics device into the second channel.

The means for amplifying the polynucleotide portion of the first and second antibody conjugates may comprise any of the methods, devices, primers and reagents disclosed in the present application for amplifying the polynucleotide portion of an antibody conjugate.

The amplification step can be performed according to any technique available in the art. The amplification step may be performed by isothermal amplification. In some embodiments, the amplification step is performed by PCR amplification. Optionally, the amplification step comprises the step of binding a first amplification primer to the first amplification sequence in the polynucleotide portion of the antibody conjugate and binding a second amplification primer to a second amplification sequence in the polynucleotide portion of the antibody conjugate.

In some embodiments, a multiplex PCR or a multiplex isothermal amplification is performed. As used herein, a multiplex PCR or a multiplex isothermal amplification refer to amplification techniques which are used to amplify or replicate multiple targets in a single step. In some embodiments, different primer sets are used to amplify the polynucleotide portions of the plurality of antibody conjugates. For example, two different primer sets can be used to amplify the polynucleotide portions of the first and second antibody conjugates. Optionally, the same set of primers are used to amplify the polynucleotide portions of the plurality of antibody conjugates. For example, a single set of primers can be used to amplify the polynucleotide portions of the first and second antibody conjugates. Those skilled in the art will be able to determine adequate conditions to perform the multiplex amplification step, such as the optimum primer length, temperature and specificity.

In some embodiments, the microfluidics device used to detect one of a plurality of markers in a sample comprises several different capture DBDs, wherein each one of the several capture DBDs recognizes and binds each of the binding nucleotide sequences present in the several antibody conjugates.

In other embodiments, the microfluidics device used to detect one of a plurality of markers in a sample comprises a single capture DBD, wherein the single capture DBD recognizes and binds every binding nucleotide sequences present in the several antibody conjugates.

As used herein, “means for contacting the amplified polynucleotides with the capture DBD or the detection DBD” refers to providing the adequate conditions and parameters so that the capture DBD or detection DBD specifically recognize and binds the amplified polynucleotides, such as the right ratio of amplified polynucleotide to capture or detection DBD, temperature, pH, buffer, etc.

In some embodiments, the means for contacting the amplified polynucleotides with the capture DBD or capture DBDs comprises moving the first and second amplified polynucleotides into a channel of the microfluidics device containing the capture DBD. Optionally, the means for contacting the amplified polynucleotides with the capture DBD or capture DBDs comprises moving the first and second amplified polynucleotides from different channels into a channel of the microfluidics device containing the capture DBD.

In some embodiments, the means for contacting the amplified polynucleotide with the capture DBD comprises moving the capture DBD or the different capture DBDs into a channel of the microfluidics device containing the first and second amplified polynucleotides. Optionally, the means for contacting the amplified polynucleotide with the capture DBD comprises moving the capture DBD or the different capture DBDs into a channel of the microfluidics device containing the first amplified polynucleotide and subsequently into a channel of the microfluidics device containing the second amplified polynucleotide.

In some embodiments, the means for contacting the amplified polynucleotides with the capture DBD or the several different capture DBDs may comprise moving the amplified polynucleotides from a first channel of the microfluidics device into a second channel of the microfluidics device and the capture DBD or capture DBDs from a third channel of the microfluidics device into the second channel Optionally, the means for contacting the sample with the amplified polynucleotides may comprise moving the capture DBD or capture DBDs from a first channel of the microfluidics device into a second channel of the microfluidics device, the first amplified polynucleotide from a third channel of the microfluidics device into the second channel and the second amplified polynucleotide from a fourth channel of the microfluidics device into the second channel.

In some embodiments, the microfluidics device comprises multiple detection DBDs, one for each of the markers to be detected. Optionally, each of the plurality of detection DBDs is released from a separate channel in the microfluidics device, i.e. the first detection DBD is released from a first channel in the microfluidics device and the second detection DBD may be released from a second channel in the microfluidics device. In some embodiments, the release of the first detection DBD from the first channel and the first detection step occur before the release of the second detection DBD from the second channel and before the second detection step. In another embodiment, the first and second or subsequent detection DBDs are released from the same channel in the microfluidics device, but at different times.

In some embodiments, the first and second or subsequent detection DBDs are attached or linked to the same detectable labels. Optionally, the first and second or subsequent detection DBDs are attached or linked to different detectable labels.

In some embodiments, if the first and second or subsequent detection DBDs are attached to the same detectable labels, each of the plurality of detection DBDs is released from a separate channel in the microfluidics device and the determination of what marker is detected may be based on timing if the release of the different detection DBDs is sequential. Optionally, each of the plurality of detection DBDs is released from a separate channel in the microfluidics device and the release is simultaneous to different channels, the determination of what marker is detected may be based on the location of said detectable label, wherein each channel comprises means for detecting the detectable labels.

In some embodiments, if the first and second or subsequent detection DBDs are attached to different detectable labels, each of the plurality of detection DBDs is released from a separate channel in the microfluidics device or optionally each of the plurality of detection DBDs is released from a separate channel in the microfluidics device. If the first and second or subsequent detection DBDs are attached to different detectable labels the microfluidics device may comprise different means for detecting the detectable labels attached to the detection DBDs.

In some embodiments, the microfluidic device comprises means for washing the antibody conjugate bound to the antigen or marker.

In some embodiments, the microfluidic device comprises means for washing the amplified polynucleotides bound to the capture DBD. The means for washing the amplified polynucleotides bound to the capture DBD may comprise moving one or more wash buffers into the channel or channels of the microfluidics device containing the amplified polynucleotides bound to the capture DBD and removing the one or more wash buffers from that channel or channels. Optionally, the means for washing the amplified polynucleotides bound to the capture DBD comprises moving the wash buffer into a channel or channels comprising the amplified polynucleotides bound to the capture DBD and removing the amplified polynucleotide bound to the capture DBD from that channel Optionally, the means for washing the amplified polynucleotides bound to the capture DBD comprises moving the amplified polynucleotides bound to the capture DBD through one or more channels comprising a wash buffer. In some embodiments, the means for washing the amplified polynucleotides bound to the capture DBD comprises moving the amplified polynucleotides bound to the capture DBD into a channel or channels comprising a wash buffer and removing the wash buffer from that channel or channels.

In some embodiments, the microfluidic device comprises means for washing the amplified polynucleotides bound to the detection DBD. The means for washing the amplified polynucleotides bound to the detection DBD may comprise moving one or more wash buffers into the channel or channels of the microfluidics device containing the amplified polynucleotides bound to the detection DBD and removing the one or more wash buffers from that channel or channels. Optionally, the means for washing the amplified polynucleotides bound to the detection DBD comprises moving the amplified polynucleotides bound to the detection DBD through one or more channels comprising a wash buffer. In some embodiments, the means for washing the amplified polynucleotides bound to the detection DBD comprises moving the amplified polynucleotides bound to the detection DBD into a channel comprising a wash buffer and removing the wash buffer from that channel Optionally, the means for washing the amplified polynucleotide bound to the detection DBD comprises moving the wash buffer into a channel comprising the amplified polynucleotides bound to the detection DBD and removing the amplified polynucleotides bound to the detection DBD from that channel.

In some embodiments, the microfluidic device comprises means for washing the immobilized marker or markers, once the marker or markers are bound to the capture molecule in the scaffold, in order to remove unbound materials present in the sample. The means for washing the immobilized marker or markers may comprise moving one or more wash buffers into the channel or channels of the microfluidics device containing the immobilized marker or markers and removing the one or more wash buffers from that channel Optionally, the means for washing the immobilized marker or markers comprises moving the immobilized marker or markers through one or more channels comprising a wash buffer. In some embodiments, the means for washing the immobilized marker or markers comprises moving the immobilized marker or markers into a channel comprising a wash buffer and removing the wash buffer from that channel Optionally, the means for washing the immobilized marker comprises moving the wash buffer into a channel or channels comprising the immobilized marker or markers and removing the immobilized marker/s from that channel or channels.

In a tenth aspect, the application relates to a microfluidics device for detecting a microorganism in a sample comprising:
(a) means for receiving the sample;
(b) means for amplifying a polynucleotide from the microorganism and introducing a first binding site that is capable of binding to a capture DNA binding domain (DBD) and is incapable of binding to a detection DBD and a second binding site that is capable of binding to the detection DBD and is incapable of binding to the capture DBD;
(c) the capture DBD affixed to a scaffold;
(d) means for contacting the amplified polynucleotide with the capture DBD;
(e) the detection DBD attached to a detectable label;
(f) means for contacting the amplified polynucleotide with the detection DBD; and
(g) means for detecting the label.

In some embodiments, the microfluidics device further comprises means for releasing a polynucleotide from the microorganism, which include extracting the polynucleotide from said microorganism and will depend on the microorganism type. In some embodiments, the means for releasing a polynucleotide from the microorganism includes a cell disruption step. In certain embodiments, the microfluidics device may comprise means for isolating or purifying the polynucleotide after the release.

In some embodiments, the means for releasing a polynucleotide from the microorganism may comprise moving the sample into a channel of the microfluidics device containing an enzymatic or chemical solution. Optionally, the means for releasing a polynucleotide from the microorganism may comprise moving the enzymatic or chemical solution into a channel of the microfluidics device containing the sample.

In some embodiments, the means for releasing a polynucleotide from the microorganism may comprise moving the sample into a channel of the microfluidics device which contains a heating element or means for releasing the polynucleotide by thermal lysis. Optionally, the means for releasing a polynucleotide from the microorganism may comprise moving the sample into a channel of the microfluidics device which contains means for mechanical disruption of the cell wall, such as a sonication element or beads to induce the cell disruption.

In some embodiments, if the genetic material of the microorganism is RNA, the microfluidics device comprises means to convert said RNA into cDNA.

In some embodiments, the microfluidics device further comprises means to isolate the polynucleotide, such as a channel to isolate or purify the released or extracted polynucleotide. The channel may comprise means for inactivation of nucleases, for example, a RNase solution can be moved into the channel where the released polynucleotide is DNA or a DNase solution can be moved into the channel where the released polynucleotide is RNA. Optionally, the released polynucleotide may be moved into a channel comprising means for inactivation of nucleases.

The means for amplifying the released polynucleotide introduces a first binding site that is capable of binding to a capture DNA binding domain (DBD) and is incapable of binding to a detection DBD and a second binding site that is capable of binding to the detection DBD and is incapable of binding to the capture DBD. The means for amplifying the released polynucleotide may comprise any of the methods, devices, primers and reagents disclosed in the present application for amplifying the polynucleotide portion of an antibody conjugate.

The amplification step can be performed in the microfluidics device according to any technique available in the art. The amplification step may be performed by isothermal amplification. In some embodiments, the amplification step is performed by PCR amplification.

The amplification step performed in the microfluidic device may introduce a capture binding site by including a tail (a fragment of polynucleotides) on the forward primer (5′ end primer) recognizing and annealing to the genetic material of the microorganism, wherein the tail does not anneal to the polynucleotides from the plurality of microorganisms. The amplification step performed in the microfluidic device may also introduce a detection binding site by including a tail (a fragment of polynucleotides) on the reverse primers (3′ end primers) recognizing the plurality of microorganisms, wherein the tail does not anneal to the polynucleotides from the plurality of microorganisms.

In an eleventh aspect, the present disclosure provides a microfluidics device for detecting one of a plurality of microorganisms in a sample comprising:
(a) means for receiving the sample;
(b) means for amplifying a polynucleotide from a plurality of microorganisms and introducing (i) a capture binding site that is capable of binding a capture DNA binding domain (DBD) and is not capable of binding any of a plurality of detection DBDs and (ii) detection binding sites that are capable of binding a plurality of detection DBDs and are incapable of binding the capture DBD;
(c) the capture DBD affixed to a scaffold;
(d) means for contacting the amplified polynucleotide with the capture DBD;
(e) the plurality of detection DBDs, wherein each detection DBD is attached to a detectable label;
(f) means for contacting the amplified polynucleotide with the plurality of detection DBDs; and
(g) means for detecting the detectable label attached to each of the plurality of detection DBDs.

In some embodiments, the microfluidic device for detecting one of a plurality of microorganisms further comprises means for releasing a polynucleotide from the microorganism, which include extracting the polynucleotide from said microorganism and will depend on the microorganism type. In some embodiments, the means for releasing a polynucleotide from the microorganism includes a cell disruption step. In certain embodiments, the microfluidics device may comprise means for isolating or purifying the polynucleotide after the release.

In some embodiments, the means for releasing a polynucleotide from the microorganism may comprise moving the sample into a channel of the microfluidics device containing an enzymatic or chemical solution. Optionally, the means for releasing a polynucleotide from the microorganism may comprise moving the enzymatic or chemical solution into a channel of the microfluidics device containing the sample.

In some embodiments, the means for releasing a polynucleotide from the microorganism may comprise moving the sample into a channel of the microfluidics device which contains means for heating the sample or a means and releasing the polynucleotide by thermal lysis. Optionally, the means for releasing a polynucleotide from the microorganism may comprise moving the sample into a channel of the microfluidics device which contains means for mechanical disruption of the cell wall, such as sonication or beads to induce the cell disruption.

In some embodiments, if the genetic material of the microorganism is RNA, the microfluidics device comprises means to convert said RNA into cDNA.

In some embodiments, the microfluidics device for detecting one of a plurality of microorganisms further comprises means for isolating the polynucleotides, such as a channel to isolate or purify the released polynucleotide. The channel may comprise means for denaturation of nucleoprotein complexes, such as a heating element and/or means for inactivation of nucleases, for example, a RNase solution can be moved into the channel where the released polynucleotide is present for RNA extraction or a DNase solution can be moved into the channel where the released polynucleotide is present for DNA extraction. Optionally, the released polynucleotide may be moved into a channel comprising a heating element and/or means for inactivation of nucleases.

The means for amplifying the released polynucleotide introduces a first binding site that is capable of binding to a capture DNA binding domain (DBD) and is incapable of binding to any of a plurality of the detection DBDs and a second binding site that is capable of binding to the plurality of the detection DBDs and is incapable of binding to the capture DBD, wherein each detection DBD can recognize and bind a specific microorganism from the plurality of microorganisms present in the sample. The means for amplifying the released polynucleotide may comprise any of the methods, devices, primers and reagents disclosed in the present application for amplifying the polynucleotide portion of an antibody conjugate.

The amplification step can be performed in the microfluidics device according to any technique available in the art. The amplification step may be performed by isothermal amplification. In some embodiments, the amplification step is performed by PCR amplification.

In some embodiments, a multiplex PCR or a multiplex isothermal amplification is performed. As used herein, a multiplex PCR or a multiplex isothermal amplification refer to amplification techniques which are used to amplify or replicate multiple targets (polynucleotides from different microorganisms) in a single step. In some embodiments, different primer sets are used to amplify the polynucleotides of the microorganism or plurality of microorganisms. For example, two or more different primer sets can be used to amplify the different microorganisms present in the sample. Optionally, the same set of primers are used to amplify the polynucleotides of the microorganism or plurality of microorganisms. Those skilled in the art will be able to determine adequate conditions to perform the multiplex amplification step in the microfluidics device, such as the optimum primer length, temperature and specificity.

The amplification step performed in the microfluidic device may introduce a capture binding site by including a fragment of polynucleotides (a tail) on the forward primers (5′ end primers) annealing to the genetic material of the plurality of microorganisms, wherein the tail does not anneal to the polynucleotides from the plurality of microorganisms. The amplification step performed in the microfluidic device may also introduce a detection binding site by including a fragment of polynucleotides (a tail) on the reverse primers (3′ end primers) annealing to the genetic material of the plurality of microorganisms, wherein the tail does not anneal to the polynucleotides from the plurality of microorganisms.

In some embodiments, the microfluidics device used to detect one of a plurality of microorganisms in a sample comprises a single capture DBD, wherein the single capture DBD recognizes and binds every capture binding nucleotide sequences which have been introduced by the amplification step performed in the microfluidics device.

As used herein, “means for contacting the amplified polynucleotides with the capture DBD” refers to providing the adequate conditions and parameters so that the capture DBD specifically recognize and binds the amplified polynucleotides, such as the right ratio of amplified polynucleotide to capture or detection DBD, temperature, pH, buffer, etc.

In some embodiments, the means for contacting the amplified polynucleotides with the capture DBD comprises moving the amplified polynucleotides into a channel of the microfluidics device containing the capture DBD. Optionally, the means for contacting the amplified polynucleotides with the capture DBD comprises moving the plurality of amplified polynucleotides from different channels into a channel of the microfluidics device containing the capture DBD.

In some embodiments, the means for contacting the amplified polynucleotide with the capture DBD comprises moving the capture DBD into a channel of the microfluidics device containing the amplified polynucleotides. Optionally, the means for contacting the amplified polynucleotide with the capture DBD comprises moving the capture DBD into a channel of the microfluidics device containing the first amplified polynucleotide and subsequently into a channel of the microfluidics device containing the second amplified polynucleotide.

In some embodiments, the means for contacting the amplified polynucleotides with the capture DBD may comprise moving the amplified polynucleotides from a first channel of the microfluidics device into a second channel of the microfluidics device and the capture DBD from a third channel of the microfluidics device into the second channel Optionally, the means for contacting the sample with the amplified polynucleotides may comprise moving the capture DBD from a first channel of the microfluidics device into a second channel of the microfluidics device, the first amplified polynucleotide from a third channel of the microfluidics device into the second channel and the second amplified polynucleotide from a fourth channel of the microfluidics device into the second channel.

In some embodiments, the microfluidics device comprises multiple detection DBDs, one for each of the plurality of microorganisms to be detected. Optionally, each of the plurality of detection DBDs is released from a separate channel in the microfluidics device, i.e. the first detection DBD is released from a first channel in the microfluidics device and the second detection DBD may be released from a second channel in the microfluidics device. In some embodiments, the release of the first detection DBD from the first channel and the first detection step occur before the release of the second detection DBD from the second channel and before the second detection step. In another embodiment, the first and second or subsequent detection DBDs are released from the same channel in the microfluidics device, but at different times.

In some embodiments, the first and second or subsequent detection DBDs are attached or linked to the same detectable labels. Optionally, the first and second or subsequent detection DBDs are attached or linked to different detectable labels.

In some embodiments, if the first and second or subsequent detection DBDs are attached to the same detectable labels, each of the plurality of detection DBDs is released from a separate channel in the microfluidics device and the determination of what microorganism is detected may be based on timing if the release of the different detection DBDs is sequential. Optionally, each of the plurality of detection DBDs is released from a separate channel in the microfluidics device and the release is simultaneous to different channels, the determination of what microorganism is detected may be based on the location of said detectable label, wherein each channel comprises means for detecting the detectable labels.

In some embodiments, if the first and second or subsequent detection DBDs are attached to different detectable labels, each of the plurality of detection DBDs is released from a separate channel in the microfluidics device or optionally each of the plurality of detection DBDs is released from a separate channel in the microfluidics device. If the first and second or subsequent detection DBDs are attached to different detectable labels the microfluidics device may comprise different means for detecting the detectable labels attached to the detection DBDs.

In some embodiments, the microfluidic device comprises means for washing the amplified polynucleotides bound to the capture DBD. The means for washing the amplified polynucleotides bound to the capture DBD may comprise moving one or more wash buffers into the channel or channels of the microfluidics device containing the amplified polynucleotides bound to the capture DBD and removing the one or more wash buffers from that channel or channels. Optionally, the means for washing the amplified polynucleotides bound to the capture DBD comprises moving the wash buffer into a channel or channels comprising the amplified polynucleotides bound to the capture DBD and removing the amplified polynucleotide bound to the capture DBD from that channel Optionally, the means for washing the amplified polynucleotides bound to the capture DBD comprises moving the amplified polynucleotides bound to the capture DBD through one or more channels comprising a wash buffer. In some embodiments, the means for washing the amplified polynucleotides bound to the capture DBD comprises moving the amplified polynucleotides bound to the capture DBD into a channel or channels comprising a wash buffer and removing the wash buffer from that channel or channels.

In some embodiments, the microfluidic device comprises means for washing the amplified polynucleotides bound to the plurality of detection DBDs. The means for washing the amplified polynucleotides bound to the plurality of detection DBDs may comprise moving one or more wash buffers into the channel or channels of the microfluidics device containing the amplified polynucleotides bound to the plurality of detection DBDs and removing the one or more wash buffers from that channel or channels. Optionally, the means for washing the amplified polynucleotides bound to the plurality of detection DBDs comprises moving the amplified polynucleotides bound to the plurality of detection DBDs through one or more channels comprising a wash buffer. In some embodiments, the means for washing the amplified polynucleotides bound to the detection DBDs comprises moving the amplified polynucleotides bound to the detection DBDs into a channel comprising a wash buffer and removing the wash buffer from that channel Optionally, the means for washing the amplified polynucleotide bound to the detection DBDs comprises moving the wash buffer into a channel comprising the amplified polynucleotides bound to the detection DBDs and removing the amplified polynucleotides bound to the detection DBDs from that channel.

The following embodiments apply to any of the above aspects for the microfluidics device.

The skilled artisan would recognize that the antibody conjugates of the microfluidics devices of the disclosure may be replaced with aptamer conjugates or a mixture of antibody conjugates and aptamer conjugates. Definitions and Examples of the antibody, antibody conjugate, aptamer, aptamer conjugates, amplification techniques, the capture DBD, the detection DBD, the detectable label, scaffolds, SAW and FET techniques, can be found in the earlier aspects of the disclosure (antibody-polynucleotide conjugate aspect and method to detect a marker or microorganism).

In some embodiments, the microfluidic device comprises multiple microfluidic channel blocks, with fluid flow between said blocks being selectively operable. Said blocks may be arranged sequentially, from a first block to subsequent downstream blocks. Alternatively, blocks may form multiple branches of microfluidic channel blocks. In a further embodiment of the present method, there may be located on the microfluidic device a valve for the control of the flow of fluid within the microfluidic device. Said control may include selectively permitting the pass or block of fluid. The valve may further allow the introduction of new materials to the microfluidic device. Further still, the valve may permit the drainage of waste material from the microfluidic device. In a further embodiment such valves may be located between adjacent microfluidic channel blocks, so as to control the flow of fluid between said blocks.

In some embodiments, the microfluidic device includes a power source. The power source may be a battery, a capacitor, a fuel cell, a solar cell. In some embodiments, the microfluidics device comprises a connector to an external power source. The connector may be a USB, USB-c, HDMI, POE, a four-pin connector, a six-pin connector, an eight-pin connector, a twenty-pin connector, and a twenty-four pin connector or any other connector to an external power source. In some embodiments, the microfluidic device comprises means for cycling an electric field or a magnetic field. Examples of means for cycling an electric field or a magnetic field are known by the skilled person in the art.

In some embodiments, the microfluidics device comprises an input/output device. Optionally, the input/output device is a screen, such as a touch screen. In some embodiments, the input/output device comprises a keyboard or one or more switches. The input/output device may comprise a light or a series of lights for signaling the detection of one or more markers or microorganisms. In some embodiments, the microfluidic device includes means for communication of the device with an input/output device. Non-limiting examples of means for communication include a USB port, a USB-c port, an HDMI port, a VGA port, an S-video port, a composite video port, an ethernet port, a firewire port, an eSATA port, a thunderbolt port, a DVI port, and a display port.

In some embodiments, the microfluidic device comprises an acoustic wave sensor configured such that the acoustic wave element is disposed on a principal surface of a piezoelectric substrate and a reactive membrane extends over the acoustic wave element. Optionally, the piezoelectric substrate is made of single-crystalline dielectric such as LiTaO3, LiNaO3, or quartz, for example. In some embodiments, the acoustic wave element includes comb-shaped IDT (interdigital transducer) electrodes arranged to excite a surface acoustic wave and also includes reflectors that are arranged on both sides of a region containing the IDT electrodes in the propagation direction of the surface acoustic wave. In one embodiment, the IDT electrodes and the reflectors are made of Al, Au, Pt, Cu, Ag, or an alloy containing these metals, for example.

In some embodiments, the microfluidics device comprises a biosensor, such as a field-effect transistor-based sensor and a communication port. In some embodiments, the field-effect transistor-based sensor comprises a field effect transistor (FET) and a biological recognition element, such as a bio-sensitive layer, which may include a chelator or derivatized chelator. The FET includes a source, a drain, a gate, and a dielectric material, at least partially interposed between the source and the gate, and at least partially interposed between the drain and the gate. The biological recognition element, in some embodiments, may be at least partially disposed upon the dielectric material and/or at least partially disposed between the source and the drain. Optionally, the FET further comprises a carbon nanotube. In some aspects, the carbon nanotube is at least partially disposed upon the dielectric material and/or at least partially disposed between the source and the drain, and the biological recognition element is at least partially disposed upon the carbon nanotube. The FET-based sensor optionally includes a sample portion configured to receive the detectable label and place the detectable label in contact with the biological recognition element. In some embodiments, the present disclosure provides a sample testing device comprising the FET-based sensor and a communication port. The sample testing device is configured to communicate, by way of the communication port, sensor data—which may be based on a signal provided by the drain and/or the source of the FET—to a computing device, such as a mobile computing device. In some embodiments, the present disclosure provides software that, when executed on the sample testing device and/or on the coupled computing device, provides a graphical user interface (GUI)—on the sample testing device and/or on the computing device—via which a user can interact with the sample testing device, for example by controlling sample tests, viewing sample test results, and/or the like. The FET-based sensor and/or the sample testing device, in various embodiments, may include a local power source, or may be powered by way of a remote power source, such as a power source included in the computing device, that may be coupled to a power port of the sample testing device and/or the FET-based sensor. The FET may be a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, the FET substrate comprises a dielectric material. Optionally, the substrate comprises Gallium Nitride.

In some embodiments, the FET comprises a chelator or a derivatized chelator. Optionally, the FET comprises a chelator or a derivatized chelator at least partially interposed between the source and the drain. In some embodiments, the chelator or the derivatized chelator is deposited on the surface of a scaffold within the microfluidics device (e.g., a FET within the microfluidics device) and is configured to contact a detectable label. Optionally, the detectable label comprises metal ion. The chelator or the derivatized chelator may be configured on the FET to selectively bind with the metal ion, such that the selective binding between the chelator or the derivatized chelator and the metal ion causes a change in an electrical current between the source and the drain. In some embodiments, the change in the electrical current is provided as output for use in at least one of detecting the metal ion, identifying the metal ion, or measuring an aspect of the metal ion. Optionally, a first electrical voltage is applied to the source and a second electrical voltage is applied to the drain, the first electrical voltage being different from the second electrical voltage, thereby contributing to the electrical current between the source and the drain.

Non-limiting examples of FET chips that can be used in the present disclosure are: a Gallium nitride (GaN) chip, a high quality Silicon Nanowire Field Effect Transistors (SiNW-FETs), a Metal Oxide Semiconductor Field Effect Transistor (MOSFET), a nanowire field-effect transistor (NWFET) chip, a carbon nanotube field-effect transistor (CNTFET) chip, an ion-sensitive field-effect transistor (ISFET) chip, an oxide-semiconductor field-effect transistor (OSFET) chip or a field-effect transistor chip fabricated by a complementary metal oxide semiconductor (CMOS) process. In some embodiments, the substrate comprises Gallium nitride. In some embodiments, the FETs have a semiconductor film (the channel) that is separated from an electrode (the gate) by a thin film insulator, made of e.g. silicon oxide, metal oxide or others. This gate-insulator-organic semiconductor sandwich is analogous to a capacitor that causes field-effect current modulation in the channel (between said source and drain electrodes which contact the semiconductor film). Hence, the current between the source and drain electrodes can be adjusted by tuning the voltage applied to the gate electrode.

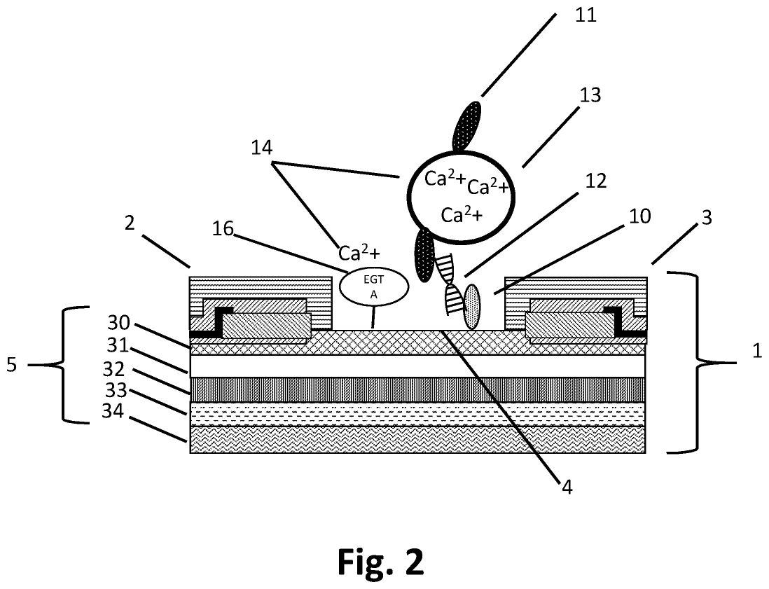

FIG. 2. shows a schematic side cross sectional representation of a transistor device (1) and liposome DBD assay in accordance with an aspect of the present disclosure. The detection is based on the release of calcium ions (Ca²⁺) (14) near the sensor-liquid-interface. The liposomes (13) containing a solution of Ca²⁺ ions are attached to the surface of a substrate (5) (comprised of layers 30, 31, 32, 33, and 34 in the example of FIG. 2), via a DBD assay consisting of a liposome (13), a detection DBD (11), an amplified polynucleotide (12), and a capture DBD (10). A calcium chelator, such as EGTA (16), ethyleneglycol-bis(β-aminoethyl)-N,N,N′,N′-tetraacetic Acid binds Ca²⁺ ions near the FET gate (4). The transistor comprises a source (2) and a drain (3) deposited onto the substrate. The substrate consists of a layer of AlGaN (30), unintentionally doped (UID) GaN (31), Carbon Doped GaN (32), AlN (33) and SiC (34).

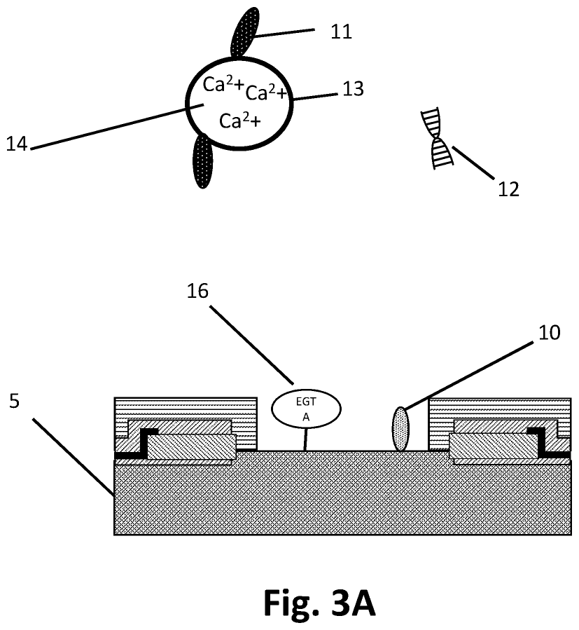

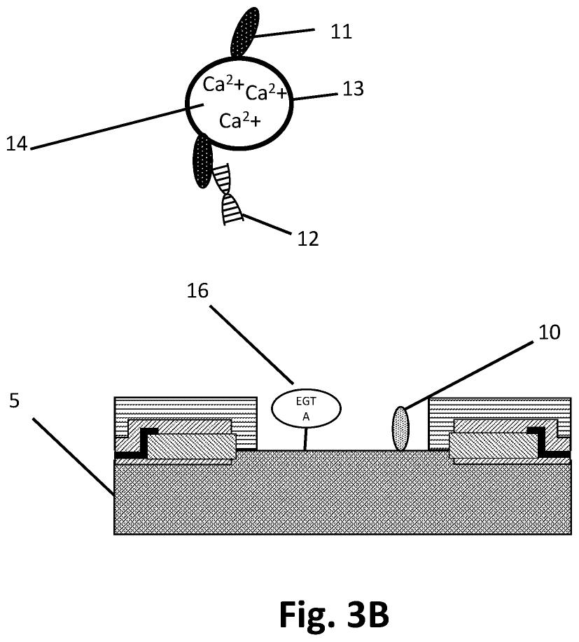

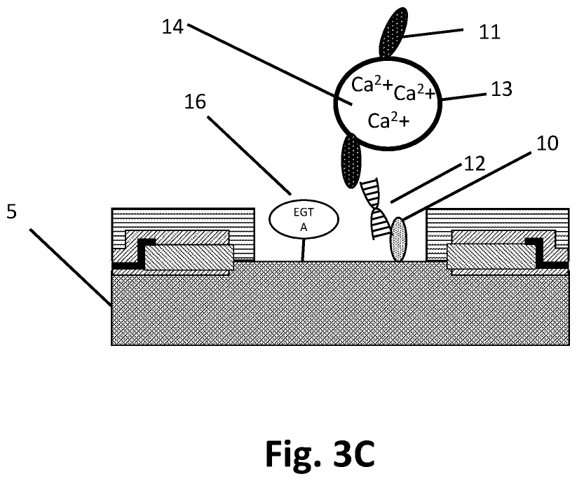

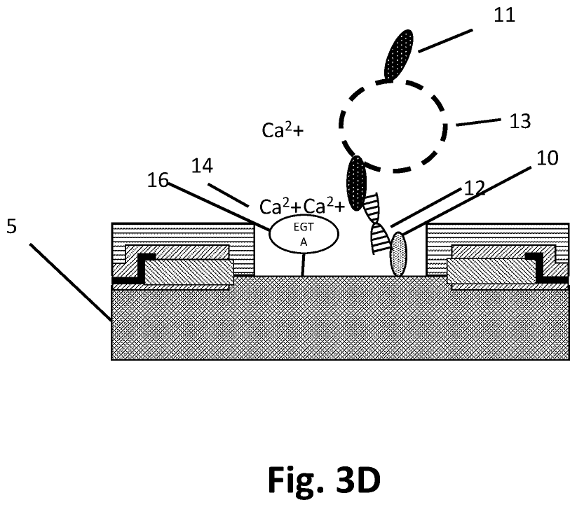

FIGS. 3A-D show side cross sectional representations of a scheme for detection of an amplified polynucleotide (12) in solution using FET transistors, such as transistor (1) described herein. FIG. 3A shows liposomes (13) containing a solution of calcium ions (14) conjugated with detection DBDs (11), and amplified polynucleotides (12) floating in a solution. The substrate (5) surface is functionalized with capture DBDs (10) and EGTA (16). FIG. 3B shows the liposome (13) and amplified polynucleotide (12) forming a DBD assay half sandwich-like structure in solution. FIG. 3C shows the formation of the DBD assay as the amplified polynucleotide (12) binds to the capture DBD (10) on the surface of the substrate (5). FIG. 3D shows the release of the calcium ions (14) from the liposome (13) which rapidly bind with the EGTA (16) and create a detectable voltage change in the transistor (1).

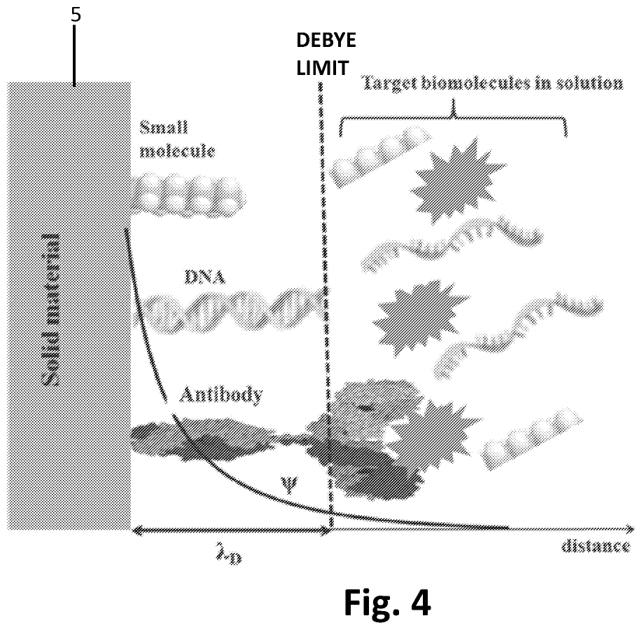

FIG. 4 shows the electrical double-layer length known as the Debye limit for materials ability to interact with a substrate interface 5 in order to make a detectable change in the device voltage.

In some embodiments, the sample is a biological sample. Optionally, the biological sample comprises one or more markers. The sample may be a biological sample into which one or more biomarkers are released, or a fluid derived from the biological sample into which one or more biomarkers are initially released. Such derivation may occur either in vivo or in vitro. In some instances, the biological sample is a circulating fluid such as blood or lymph, or a fraction thereof, such as serum or plasma. In other embodiments, the biological sample remains substantially in a particular locus, for example, synovial fluid, cerebrospinal fluid or interstitial fluid. Optionally, the biological sample is an excreted fluid, for example, urine, breast milk, saliva, sweat, tears, mucous, nipple aspirants, semen, vaginal fluid, pre-ejaculate and the like. A biological sample may also comprise a liquid in which cells are cultured in vitro such as a growth medium, or a liquid in which a cell sample is homogenized, such as a buffer. In some embodiments, the sample is a food sample. Optionally, the sample is an environmental sample, such as a water or a soil sample, which contains markers or molecules to be detected. The sample may contain an allergen or a microorganism. In some embodiments, the microorganism is selected from the group of a bacterium, a fungus, an archaeon, an alga, a protozoan and a virus.

Examples of markers or biomarkers which can be detected according to the methods of the present disclosure include proteins, lipids, lipoproteins, glycoproteins, nucleic acids (including circulating nucleic acids), carbohydrates, lipopolysaccharides, small molecule metabolites, and fragments thereof. In some embodiments, the marker is a biomarker, an environmental marker, an allergen, or a microorganism (e.g. a bacterium, a fungus, an archaeon, an alga, a protozoan and a virus). The marker or biomarker may be present in the sample at a concentration that cannot be detected without signal amplification. Typically, the presence and/or the concentration of a biomarker (or biomarkers, or pattern or patterns of biomarkers) in a sample is discriminatory between physiological and pathological states of the cells from which they are released.

In some embodiments, the means for receiving the sample in the microfluidics device is a reservoir in the device, wherein the sample is loaded into said sample reservoir, in order to have it tested. Optionally, the means for receiving the sample is an injection port.

The means for amplifying the polynucleotide portion of the antibody conjugate or aptamer conjugate may comprise any of the methods, devices, primers and reagents disclosed in the present application for amplifying the polynucleotide portion of an antibody conjugate or aptamer conjugate.

The amplification step can be performed according to any technique available in the art. The amplification step may be performed by isothermal amplification. In some embodiments, the amplification step is performed by PCR amplification.

In some embodiments, the capture DBD is affixed to a scaffold in the microfluidics device. Examples of scaffolds have been already disclosed in the present application. Optionally, the capture DBD is capable of being affixed to a scaffold in the microfluidics device. For example, the capture DBD may be linked to a magnetic bead or a metallic particle, which will bind the scaffold upon the application of an electric current or a magnetic field. In such embodiments, the cycling of the electric current or the magnetic field can be used to mix the solutions within the microfluidics device. Optionally, the scaffold is a detector for the detectable label. In some embodiments, the scaffold is adjacent to the detector for the detectable label. In some embodiments, the detection step comprises the step of transporting the detectable label to a detector for the detectable label.

As used herein, “means for contacting the amplified polynucleotide with the capture DBD or the detection DBD” refers to providing the adequate conditions and parameters so that the capture DBD or detection DBD specifically recognize and binds the amplified polynucleotide, such as the right ratio of amplified polynucleotide to capture or detection DBD, temperature, pH, buffer, etc.

In some embodiments, the means for contacting the amplified polynucleotide with the capture DBD comprises moving the amplified polynucleotide into a channel of the microfluidics device containing the capture DBD. Optionally, the means for contacting the amplified polynucleotide with the capture DBD comprises moving the capture DBD into a channel of the microfluidics device containing the amplified polynucleotide. The means for contacting the amplified polynucleotide with the capture DBD may comprise simultaneously moving the amplified polynucleotide from a first channel of the microfluidics device into a second channel of the microfluidics device and the capture DBD from a third channel of the microfluidics device into the second channel.

In some embodiments, the means for contacting the amplified polynucleotide with the detection DBD comprises moving the amplified polynucleotide into a channel of the microfluidics device containing the detection DBD. Optionally, the means for contacting the amplified polynucleotide with the detection DBD comprises moving the detection DBD into a channel of the microfluidics device containing the amplified polynucleotide. The means for contacting the amplified polynucleotide with the detection DBD may comprise simultaneously moving the amplified polynucleotide from a first channel of the microfluidics device into a second channel of the microfluidics device and the detection DBD from a third channel of the microfluidics device into the second channel.

In some embodiments, the microfluidic device comprises means for washing the amplified polynucleotide bound to the capture DBD. The means for washing the amplified polynucleotide bound to the capture DBD may comprise moving one or more wash buffers into the channel of the microfluidics device containing the amplified polynucleotide bound to the capture DBD and removing the one or more wash buffers from that channel Optionally, the means for washing the amplified polynucleotide bound to the capture DBD comprises moving the amplified polynucleotide bound to the capture DBD through one or more channels comprising a wash buffer. In some embodiments, the means for washing the amplified polynucleotide bound to the capture DBD comprises moving the amplified polynucleotide bound to the capture DBD into a channel comprising a wash buffer and removing the wash buffer from that channel Optionally, the means for washing the amplified polynucleotide bound to the capture DBD comprises moving the wash buffer into a channel comprising the amplified polynucleotide bound to the capture DBD and removing the amplified polynucleotide bound to the capture DBD from that channel.

In some embodiments, the microfluidic device comprises means for washing the amplified polynucleotide bound to the detection DBD. The means for washing the amplified polynucleotide bound to the detection DBD may comprise moving one or more wash buffers into the channel of the microfluidics device containing the amplified polynucleotide bound to the detection DBD and removing the one or more wash buffers from that channel Optionally, the means for washing the amplified polynucleotide bound to the detection DBD comprises moving the amplified polynucleotide bound to the detection DBD through one or more channels comprising a wash buffer. In some embodiments, the means for washing the amplified polynucleotide bound to the detection DBD comprises moving the amplified polynucleotide bound to the detection DBD into a channel comprising a wash buffer and removing the wash buffer from that channel Optionally, the means for washing the amplified polynucleotide bound to the detection DBD comprises moving the wash buffer into a channel comprising the amplified polynucleotide bound to the detection DBD and removing the amplified polynucleotide bound to the detection DBD from that channel.

In some embodiments, the means for detecting the detectable label comprises any of fluorescence, luminescence, colorimetry, radioactivity, Surface Acoustic Wave (SAW) or Surface Generated Acoustic Wave (SGAW) and Field Effect Transistor (FET) detectors disclosed herein.

Non-limiting examples of detectable labels include fluorescent labels, fluorogenic labels, dyes, colorimetric labels, radioactive labels, luminescent labels, chemiluminescent labels, magnetic particles, metal particles, particles of 1 pg or greater, charged particles, charged solution, spores and enzymatic labels or combinations thereof or any other label which is suitable to be detected by the selected technique. Optionally, the charged solution is an ionic solution. In some embodiments, the ionic solution comprises metal ions.

To bring metal ions near the surface of the channel or gate, the chelator or derivatized chelator may be conjugated to the substrate surface and binds metal ions near the FET gate. In some embodiments, the FET is a chelator-coated FET as described in US 62/718,632, U.S. 62/886,759 and PCT/US2019/046568, each of which is incorporated by reference herein in its entirety.

In some embodiments, the ionic solution comprises a metal ion. Optionally, the metal ion is a divalent or trivalent ion. The metal ion may be selected from the group consisting of Ca²⁺, Fe²⁺, Fe“, Mg Mn²⁺, Cu²⁺, Cu”, Co²⁺ and a heavy metal ion. In some embodiments, the heavy metal ion is selected from the group consisting of As⁺³, Hg⁺², Sb⁺³, and Au⁺. In some embodiments, the ionic solution comprises a cation selected from the group consisting of Ca²⁺, Fe²⁺, Fe³⁺, Mg²⁺, Mn²⁺, Cu²⁺, Cu³⁺, Zn²⁺, Ni²⁺, Co²⁺, As⁺³, Hg⁺², Sb⁺³, and Au⁺. Optionally, the ionic solution comprises Ca²⁺.

In some embodiments, the detectable label may be detected by a surface acoustic wave device, wherein the detectable label is selected from the group consisting of a magnetic particle, a metal particle, a particle of 1 pg or greater, a charged particle and a spore or a combination thereof.

In some embodiments, the detectable label may be detected by a field effect transistor, wherein the detectable label is selected from the group consisting of a magnetic particle, a metal particle, a charged particle and an ionic solution or a combination thereof. In some embodiments, the detectable label comprises an ionic solution. Optionally, the ionic solution comprises a metal ion. Non-limiting examples of metal ions include iron ions, copper ions, cobalt ions, manganese ions, chromium ions, nickel ions, zinc ions, cadmium ions, molybdenum ions, lead ions, and the like. In any of the methods disclosed herein, the metal ion being detected is, optionally, the metal ion is selected from the group consisting of Ca²⁺, Fe²⁺, Fe³⁺, Mg²⁺, Mn²⁺, Cu²⁺, Cu³⁺, Zn²⁺, Ni²⁺, Co²⁺ and a heavy metal ion (e.g., As⁺³, Hg⁺², Sb⁺³, and Au⁺). Preferably, the metal ions to be detected are divalent and trivalent ions.

In some embodiments, the detectable label is comprised within a lipid vesicle. In some embodiments, the ionic solution is comprised within a lipid vesicle. In some embodiments, the ionic solution comprises a metal ion, which is released upon disruption of the lipid vesicle.

In some embodiments, when the detectable label comprises a metal ion, the method further comprises contacting the released metal ions with a metal ion chelator or metal ion derivatized chelator located at or near the detector. In some embodiments, the metal ion chelator is attached to the surface of the detector. In some embodiments, the detector is a chelator-coated FET, such as those described in U.S. Provisional Application No. 62/718,632, U.S. Provisional Application No. 62/886,759 and PCT/US2019/046568, each of which is incorporated by reference herein in its entirety.

In some embodiments, chelating agents of metallic ions include chelating agents of Ca²⁺, Fe²⁺, Fe³⁺, Mg²⁺, Mn²⁺, Cu²⁺, Cu³⁺, Zn²⁺, Ni²⁺, Co²⁺, heavy metal ions (e.g., As⁺³, Hg⁺², Sb⁺³, and AO, and the like. It is within the skill of the art to select a chelating agent or derivatized chelating agent that will bind or complex with a particular ion of interest. See, e.g., Bers D. M., MacLeod K. T. (1988) Calcium Chelators and Calcium Ionophores. In: Baker P. F. (eds) Calcium in Drug Actions. Handbook of Experimental Pharmacology, vol 83. Springer, Berlin, Heidelberg; Hatcher, H C. et al. Future Med Chem. 2009 December; 1(9): 10.4155; Sheth, S., Curr Opin Hematol 2014, 21:179; Missy P. et al. Hum Exp Toxicol., 2000, vol. 19(8): 448-456; Sigma Aldrich, BioUltra Reagents: Chelators (available at https://www.sigmaaldrich.com/life-science/metabolomics/bioultra-reagents/chelators.html); Santa Cruz Biotechnology Chelators (available at https://www.scbt.com/scbt/browse/chelators/_/N-1azot5l); Lawson M K, et al. Curr Pharmacol Rep (2016) 2:271-280; Radford and Lippard, Curr Opin Chem Biol. 2013 April; 17(2): 129-136; Chaitman, M. et al., P T. 2016 January; 41(1): 43-50, each of which is incorporated herein in its entirety.

In some embodiments, the microfluidics device used to detect a marker in a sample comprises:
i. a first chamber for containing a plurality of antibody conjugates in a buffer, each comprising a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD, and an antibody specific for the marker;
ii. an amplification chamber in which the marker-bound antibody conjugates in the buffer are immobilized by being attached to the surface by means of a plurality of capture antibodies;
iii. a second chamber for holding and selectively providing a washing buffer to the buffer in the amplification chamber;
iv. a third chamber for holding and selectively providing a polymerase and primers to the buffer in the amplification chamber to provide amplified polynucleotides comprising the first and sequence nucleotide binding sequences;
v. means for performing convection enhanced delivery by recirculating a buffer multiple times through a fluidic circuit including the amplification chamber to reduce time required to saturate the capture antibodies with markers from diffusive timescales to convective timescales;
vi. a testing chamber in which the amplified polynucleotides are immobilized by being attached to the surface by means of a plurality of capture DBDs binding to the first nucleotide binding sequence;
vii. a fourth chamber for holding and selectively providing detection DBDs, which are capable of binding to the second nucleotide binding sequence and are conjugated to a detectable label;
viii. a fifth chamber for holding and selectively providing a washing buffer to the buffer in the testing chamber; and
ix. a detector for the detectable label.

In some embodiments, the microfluidics device provides an improved limit of detection (LOD) and comprises:
i. a first chamber for containing a plurality of antibody conjugates in a buffer, each comprising a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD, and an antibody specific for the marker;
ii. an amplification chamber in which the marker-bound antibody conjugates in the buffer are immobilized by being attached to the surface by means of a plurality of capture antibodies;
iii. a second chamber for holding and selectively providing a washing buffer to the buffer in the amplification chamber;
iv. a third chamber for holding and selectively providing a polymerase and primers to the buffer in the amplification chamber to provide amplified polynucleotides comprising the first and sequence nucleotide binding sequences;
v. means for performing convection enhanced delivery by recirculating a buffer multiple times through a fluidic circuit including the amplification chamber to reduce time required to saturate the capture antibodies with markers from diffusive timescales to convective timescales;
vi. a testing chamber in which the amplified polynucleotides are immobilized by being attached to the surface by means of a plurality of capture DBDs binding to the first nucleotide binding sequence;
vii. a fourth chamber for holding and selectively providing detection DBDs, which are capable of binding to the second nucleotide binding sequence and are conjugated to a detectable label;
viii. a fifth chamber for holding and selectively providing a washing buffer to the buffer in the testing chamber; and
ix. a detector for the detectable label.

In some embodiments, the microfluidics device comprises a sixth chamber for holding a selectively providing an activator of detectable label. Optionally, the detectable label is an ion-containing lipid vesicle, the sixth chamber comprise a vesicle-disrupting solution, and microfluids device further comprises:
i. an electrode isolated and covered with pure water as a continuous reference value subtracted from the measured conductivity, impedance or resistivity of the buffer to establish a delta value;
ii. a microcontroller which receives a delta value for analysis to generate a time coefficient (τ) for use in establishing reaction kinetics (k+/−); and
iii. a first electrode disposed in the buffer, sensing current in a second electrode disposed in the buffer according a magnitude of ions released from the lipid vesicles into the buffer, and amplifying and/or signal conditioning the sensed current for output to a detector.

Optionally, the vesicle-disrupting solution comprises a detergent. In some embodiments, the vesicle-disrupting solution comprises a vesicle-disrupting enzyme.

In some embodiments, the amplification chamber and the testing chamber are the same, and the capture antibodies and the capture DBDs are affixed to different surfaces in the chamber. Optionally, the amplification chamber is different than the testing chamber, and the microfluidics device further comprise a fluid circuit for transporting the amplified polynucleotides from the amplification chamber to the testing chamber.

In some embodiments, the amplified polynucleotides are immobilized by the capture DBDs before the detection DBDs are provided by the fourth chamber. Optionally, the detection DBDs are provided by the fourth chamber before the amplified polynucleotides are immobilized by the capture DBDs. In some embodiments, the detection DBDs are provided from the fourth chamber to the amplification chamber before the amplified polynucleotides are transported to the testing chamber. Optionally, the polynucleotide-bound detection DBDs are transported from the amplification chamber to the testing chamber. In some embodiments, the detection DBDs are provided by the fourth chamber to the testing chamber. Optionally, the detection DBDs are provided by the fourth chamber to the testing chamber before the amplified polynucleotides are transported to the testing chamber. In some embodiments, the detection DBDs are provided by the fourth chamber to the testing chamber after the amplified polynucleotides are transported to the testing chamber.

In some embodiments, the microfluidics device further comprises a sample port, such as a one-way fluidic port. Optionally, the sample port comprises a septum. In some embodiments, the sample port is configured to introduce a sample into the first chamber. Optionally, the antibody conjugates bind a marker in the sample in the first chamber, and the marker-bound antibody conjugates are transported to the amplification chamber and the marker-bound antibody conjugates are immobilized by the capture antibodies. In some embodiments, the sample port is configured to introduce a sample into the amplification chamber. Optionally, the capture antibodies bind a marker in the sample in the amplification chamber and then antibody conjugates are transported from the first chamber to the amplification chamber and bind the immobilized marker.

In some embodiments, the microfluidics device comprises an amplification chamber which comprises a scaffold to which the marker-bound antibody conjugates are selectively attached. In some embodiments, the scaffold is an immobilized surface. In some embodiments, the marker-bound antibody conjugates are attached to a plurality of capture molecules (e.g., capture antibodies).

In some embodiments, the microfluidics device further comprises means for performing convection enhanced delivery by recirculating a buffer multiple times through a fluidic circuit including the amplification chamber to reduce time required to saturate the capture molecules (e.g., capture antibodies) with markers from diffusive timescales to convective timescales. Optionally, the microfluidics device further comprises means for performing convection enhanced delivery by recirculating a buffer multiple times through a fluidic circuit including the amplification chamber to reduce time required to saturate the capture molecules (e.g., capture antibodies) with marker-bound antibody conjugates from diffusive timescales to convective timescales. In some embodiments, the microfluidics device further comprises means for performing convection enhanced delivery by recirculating a buffer multiple times through a fluidic circuit including the testing chamber to reduce time required to saturate the capture DBDs with amplified polynucleotides from diffusive timescales to convective timescales. Optionally, the microfluidics device further comprises means for performing convection enhanced delivery by recirculating a buffer multiple times through a fluidic circuit including the testing chamber to reduce time required to saturate the capture DBDs with polynucleotide-bound detection DBDs from diffusive timescales to convective timescales.

1. An antibody conjugate comprising an antibody linked to a polynucleotide, wherein the polynucleotide comprises:
a first binding nucleotide sequence that is capable of binding to a capture DNA binding domain (DBD) but incapable of binding to a detection DBD; and
a second binding nucleotide sequence that is capable of binding to the detection DBD but incapable of binding to the capture DBD.

2.-17. (canceled)

18. The antibody conjugate according to claim 1, wherein the first binding nucleotide sequence is separated from the second binding nucleotide sequence by a linker sufficient to avoid steric hindrance between the capture DBD and the detection DBD.

19. (canceled)

20. The antibody conjugate according to claim 1, wherein the polynucleotide further comprises a first amplification nucleotide sequence and a second amplification nucleotide sequence, wherein the first binding nucleotide sequence and the second binding nucleotide sequence are between the first amplification nucleotide sequence and the second amplification nucleotide sequence.

21.-22. (canceled)

23. The antibody conjugate according to claim 1, wherein the polynucleotide is capable of being released from the antibody upon antigen binding.

23. (canceled)

24. The antibody conjugate according to claim 1, further comprising a protein and wherein the antibody is linked to the polynucleotide through the protein.

25. (canceled)

26. A method for detecting a marker in a sample, the method comprising:
(a) contacting the sample with the antibody conjugate which comprises an antibody linked to a polynucleotide, wherein the polynucleotide comprises:
a first binding nucleotide sequence that is capable of binding to a capture DNA binding domain (DBD) but incapable of binding to a detection DBD; and
a second binding nucleotide sequence that is capable of binding to the detection DBD but incapable of binding to the capture DBD, wherein the antibody portion of the conjugate binds to the marker;
(b) amplifying the polynucleotide portion of the antibody conjugate;
(c) binding the first nucleotide binding sequence of the amplified polynucleotide to a capture DNA binding domain (DBD), wherein the capture DBD is affixed to a scaffold;
(d) binding the second nucleotide binding sequence of the amplified polynucleotide to a detection DBD, wherein the detection DBD is affixed to a detectable label; and
(e) detecting the detectable label.

27.-29. (canceled)

30. The method according to claim 26, wherein the polynucleotide is released upon binding of the marker to the antibody conjugate.

31. The method according to claim 26, wherein the marker is bound to a capture molecule, wherein the capture molecule is affixed to a scaffold or capable of being affixed to a scaffold.

32. The method according to claim 31, wherein the method further comprises the step of washing the capture molecule-bound marker prior to contacting the sample with the antibody conjugate.

33.-51. (canceled)

52. A method of detecting one of a plurality of markers in a sample, the method comprising:
(a) contacting the sample with a first antibody conjugate and a second antibody conjugate, wherein the first antibody conjugate and second antibody conjugate are antibody conjugates each including a corresponding antibody linked to a corresponding polynucleotide, wherein the corresponding polynucleotide comprises
a first corresponding binding nucleotide sequence that is capable of binding to a corresponding capture DNA binding domain (DBD) but incapable of binding to a corresponding detection DBD; and
a second corresponding binding nucleotide sequence that is capable of binding to the corresponding detection DBD but incapable of binding to the corresponding capture DBD,
wherein the antibody portion of the first antibody conjugate binds a different marker of the plurality of markers than another one of the plurality of markers which binds to antibody portion of the second antibody conjugate,
wherein the first binding nucleotide sequence of the polynucleotide portion of the first antibody conjugate binds to the first corresponding capture DNA binding domain (DBD) and the first binding nucleotide sequence of the polynucleotide portion of the second antibody conjugate binds to the corresponding second capture DBD, and
wherein the second binding nucleotide sequence of the polynucleotide portion of the first antibody conjugate binds to the corresponding first detection DBD, and the second binding nucleotide sequence of the polynucleotide portion of the second antibody conjugate binds to the corresponding second detection DBD;
(b) amplifying the polynucleotide portions of the marker-bound antibody conjugates;
(c) binding the first nucleotide binding sequences of the amplified polynucleotides to corresponding capture DBDs, wherein the corresponding capture DBDs bind& to the first binding nucleotide sequence of the first and second antibody conjugates and wherein each is affixed to a corresponding scaffold;
(d) binding the second binding nucleotide sequences of the amplified polynucleotides to a first corresponding detection DBDs, wherein the first corresponding detection DBDs binds to the second binding nucleotide sequences of the first antibody conjugates and wherein each is affixed to a first corresponding detectable label;
(e) performing a first detection step to detect the first detectable labels;
(f) binding the amplified polynucleotides to a second corresponding detection DBDs, wherein the second corresponding detection DBDs binds to the second binding nucleotide sequences of the second antibody conjugates and wherein each is affixed to a second corresponding detectable label; and
(g) performing a second detection step to detect the second detectable labels.

53. The method according to claim 52, wherein the first and second corresponding capture DBDs bind the same first corresponding binding nucleotide sequence.

54. The method according to claim 52, wherein the first and second corresponding capture DBDs are the same DBD.

55. The method according to claim 52, wherein the first and second corresponding capture DBDs bind different first binding nucleotide sequences.

56. The method according claim 52, wherein the corresponding polynucleotide portion of each corresponding antibody conjugate is released upon binding of the corresponding marker to the corresponding antibody conjugate.

57. The method according to claim 52, wherein each of the plurality of markers is bound to a corresponding capture molecule, wherein each corresponding capture molecule is affixed to a corresponding scaffold or capable of being affixed to a corresponding scaffold.

58. The method according to claim 57, wherein the method further comprises the step of washing the capture molecule-bound markers prior to contacting the sample with the antibody conjugates or washing the antibody conjugate-bound markers before the amplification step.

59.-96. (canceled)

97. The method according to claim 52, wherein the method further comprises:
(1) contacting the sample with a plurality of antibody conjugates, wherein each of the plurality of antibody conjugates includes, a corresponding antibody linked to a corresponding polynucleotide, wherein the corresponding polynucleotide comprises:
a first corresponding binding nucleotide sequence that is capable of binding to a corresponding capture DNA binding domain (DBD) but incapable of binding to a corresponding detection DBD, and
a second corresponding binding nucleotide sequence that is capable of binding to the corresponding detection DBD but incapable of binding to the corresponding capture DBD,
wherein each of the antibodies of the plurality of antibody conjugates binds a different marker than the corresponding antibodies of each of the other ones of the plurality of antibody conjugates, wherein the first binding nucleotide sequence of each of the plurality of antibody conjugates binds to the same or different capture DBD as the first binding nucleotide sequence of a first and a second one of the plurality of antibody conjugates, wherein the second nucleotide binding sequence of each of the plurality of antibody conjugates binds to the same or a different DBD than the second nucleotide binding sequence of the first one of the plurality of antibody conjugates, than the second one of the plurality of antibody conjugates and than the other ones of the plurality of antibody conjugates;
(2) binding the amplified polynucleotide to one or more of the DBDs, wherein each of the DBDs binds to the second binding nucleotide sequence of the antibody conjugates and is affixed to one or more detectable labels; and
(3) performing one or more detection steps to detect the one or more detectable labels.

98. (canceled)

99. A microfluidics system comprising:
(a) means for receiving a sample;
(b) an antibody conjugate, wherein the conjugate comprises an antibody linked to a polynucleotide, wherein the polynucleotide comprises a first binding nucleotide sequence that is capable of binding to a capture DNA binding domain (DBD) and is incapable of binding to a detection DBD and a second binding nucleotide sequence that is capable of binding to the detection DBD and is incapable of binding to the capture DBD;
(c) means for contacting the sample with the antibody conjugate;
(d) means for amplifying the polynucleotide portion of the antibody conjugate;
(e) the capture DBD being affixed to a scaffold;
(f) means for contacting the amplified polynucleotide with the capture DBD;
(g) the detection DBD being attached to a detectable label, wherein the capture DBD and the detection DBD are different;
(h) means for contacting the amplified polynucleotide with the detection DBD; and
(i) means for detecting the detectable label.

100. (canceled)

101. The microfluidics system according to claim 99, wherein the antibody conjugate is an antibody conjugate comprises an antibody linked to a polynucleotide, wherein the polynucleotide comprises:
a first binding nucleotide sequence that is capable of binding to a capture DNA binding domain (DBD) but incapable of binding to a detection DBD, and
a second binding nucleotide sequence that is capable of binding to the detection DBD but incapable of binding to the capture DBD.

102-117. (canceled)

118. The microfluidics system according to claim 99, wherein the system further comprises means for washing the amplified polynucleotide bound to the capture DBD and/or for washing the amplified polynucleotide bound to the detection DBD.

119. (canceled)

120. The microfluidics system according to claim 99, wherein the system further comprises means for immobilizing the marker.

121. The microfluidics system according to claim 120, wherein the means for immobilizing the marker is an antibody that binds the marker and is affixed to a solid phase or is capable of being affixed to a solid phase.

122. The microfluidics system according to claim 120, wherein the system further comprises means for washing the immobilized marker.

123. (canceled)

124. A microfluidics system comprising:
(a) means for receiving a sample;
(b) a first antibody conjugate, wherein the first antibody conjugate comprises a first antibody linked to a first polynucleotide, wherein the first polynucleotide comprises a capture nucleotide sequence that is capable of binding to a capture DNA binding domain (DBD) and is incapable of binding to a first or second detection DBD and a first detection nucleotide sequence that is capable of binding to the first detection DBD and is incapable of binding to the capture DBD or the second detection DBD;
(c) a second antibody conjugate, wherein the second antibody conjugate comprises a second antibody linked to a second polynucleotide, wherein the second polynucleotide comprises a capture nucleotide sequence that is capable of binding to the capture DBD and is incapable of binding to the first or second detection DBD and a second detection nucleotide sequence that is capable of binding to the second detection DBD and is incapable of binding to the capture DBD or the first detection DBD;
(d) means for contacting the sample with the antibody conjugates;
(e) means for amplifying the polynucleotide portions of the first and second antibody conjugates;
(f) the capture DBD affixed to a scaffold;
(g) means for contacting the amplified polynucleotides with the capture DBD;
(h) the first detection DBD attached to a first detectable label, the second detection DBD attached to a second detectable label;
(i) means for contacting the amplified polynucleotides with the first and second detection DBDs; and
(j) means for detecting the first and second detectable labels.

125. The microfluidics system according to claim 124, wherein the first antibody conjugate is an antibody conjugate comprising an antibody linked to a polynucleotide, wherein the polynucleotide comprises:
a first binding nucleotide sequence that is capable of binding to a capture DNA binding domain (DBD) but incapable of binding to a detection DBD, and
a second binding nucleotide sequence that is capable of binding to the detection DBD but incapable of binding to the capture DBD.

126. The microfluidics system according to claim 124, wherein the second antibody conjugate is an antibody conjugate comprising an antibody linked to a polynucleotide, wherein the polynucleotide comprises:
a first binding nucleotide sequence that is capable of binding to a capture DNA binding domain (DBD) but incapable of binding to a detection DBD, and
a second binding nucleotide sequence that is capable of binding to the detection DBD but incapable of binding to the capture DBD.

127.-132. (canceled)

133. The microfluidics system according to claim 132, wherein the first detection DBD is released from the first channel and the means for detecting the first detectable label is performed prior to the release of the second detection DBD from the second channel.

134.-162. (canceled)

163. The microfluidics system according to claim 124, wherein the microfluidics system comprises one or more additional antibody conjugates, wherein each of the one or more additional antibody conjugates comprises an antibody linked to one or more additional polynucleotides, wherein each of the one or more additional polynucleotides comprises a capture nucleotide sequence that binds to the capture DBD and does not bind to the first, second, or any additional detection DBD, and an additional detection nucleotide sequence that binds to the one or more additional detection DBDs and does not bind to the capture DBD, the first, second or any other additional detection DBD.

164. The microfluidics system of claim 124, wherein the system further comprises means for washing the amplified polynucleotide bound to the capture DBD or comprises means for washing the amplified polynucleotide bound to the first detection DBD, means for washing the amplified polynucleotide bound to the second detection DBD, or means for washing the amplified polynucleotide bound to the one or more additional detection DBDs, or means for washing the amplified polynucleotide bound to the one or more additional detection DBDs.

165.-167. (canceled)

168. The microfluidics system according to claim 124, wherein the system further comprises means for immobilizing the marker.

169. (canceled)

170. The microfluidics system according to claim 168, wherein the system further comprises means for washing the immobilized marker.

171.-259. (canceled)
